(19) United States
(12) Patent Application Publication
(10) Pub. No.: US 2012/0022121 A1
(43) Pub. Date: Jan. 26, 2012

(54) INDOLES, DERIVATIVES AND ANALOGS THEREOF AND USES THEREFOR

(76) Inventors: James T. DALTON, Lakeland, TN (US); Duane D. MILLER, Germantown, TN (US); Sunjoo AHN, Memphis, TN (US); Charles DUKE, Memphis, TN (US); Dong Jin HWANG, Arlington, TN (US); Jun YANG, Rockville, MD (US)

(21) Appl. No.: 13/190,150

(22) Filed: Jul. 25, 2011

Related U.S. Application Data
(63) Continuation-in-part of application No. 11/947,668, filed on Nov. 29, 2007.

(51) Int. Cl. A61K 31/404; C07D 209/12; C07D 277/64; C07D 263/57; A61P 35/04; A61K 31/4184; A61K 31/423; A61K 31/428; A61P 35/00; C07D 403/10; C07D 235/22

(52) U.S. Cl. 514/367; 548/455; 514/414; 548/491; 514/419; 548/180; 548/217; 548/309.4; 514/395; 514/375

(57) ABSTRACT

Indole derivatives and analogous compounds and pharmaceutical compositions comprising the same are provided. Also provided are methods of using these compounds to inhibit tubulin polymerization in a cell associated with a proliferative disease or to treat cancer, metastatic cancer, resistant cancer or multidrug resistant cancer, including inter-alia: prostate cancer, breast cancer, melanoma, colon cancer and bladder cancer.

### CROSS REFERENCE TO RELATED APPLICATIONS

This application is a Continuation-In-Part Application of U.S. patent application Ser. No. 11/947,668, filed Nov. 29, 2007, which is hereby incorporated by reference in its entirety.

### GOVERNMENT INTEREST STATEMENT

This invention was produced in part using funds obtained through Grant DK-065227-02 from the National Institutes of Health. Consequently, the federal government has certain rights in this invention.

### BACKGROUND OF THE INVENTION

Cancer is the second most common cause of death in the United States, exceeded only by heart disease. In the United States, cancer accounts for 1 of every 4 deaths. The 5-year relative survival rate for all cancer patients diagnosed in 1996-2003 is 66%, up from 50% in 1975-1977 (Cancer Facts & Figures American Cancer Society: Atlanta, Ga. (2008)). This improvement in survival reflects progress in diagnosing at an earlier stage and improvements in treatment. Discovering highly effective anticancer agents with low toxicity is a primary goal of cancer research.

Prostate cancer is one of the most frequently diagnosed noncutaneous cancers among men in the US and is the second most common cause of cancer deaths with over 180,000 new cases and almost 29,000 deaths expected this year. Patients with advanced prostate cancer undergo androgen deprivation therapy (ADT), typically either by luteinizing hormone releasing hormone (LHRH) agonists or by bilateral orchiectomy. Androgen deprivation therapy not only reduces testosterone, but estrogen levels are also lower since estrogen is derived from the aromatization of testosterone, which levels are depleted by ADT. Androgen deprivation therapy-induced estrogen deficiency causes significant side effects which include hot flushes, gynecomastia and mastalgia, bone loss, decreases in bone quality and strength, osteoporosis and life-threatening fractures, adverse lipid changes and higher cardiovascular disease and myocardial infarction, and depression and other mood changes.

Malignant melanoma is the most dangerous form of skin cancer, accounting for about 75% of skin cancer deaths. The incidence of melanoma is rising steadily in Western populations. The number of cases has doubled in the past 20 years. Around 160,000 new cases of melanoma are diagnosed worldwide each year, and it is more frequent in males and Caucasians. According to a WHO Report, about 48,000 melanoma-related deaths occur worldwide per year.

Currently there is no effective way to treat metastatic melanoma. It is highly resistant to current chemotherapy, radiotherapy, and immunotherapy. Metastatic melanoma has a very poor prognosis, with a median survival rate of 6 months and a 5-year survival rate of less than 5%. In the past 30 years, dacarbazine (DTIC) is the only FDA-approved drug for metastatic melanoma. However, it provides only less than 5% of complete remission in patients. In recent years, great efforts have been attempted in fighting metastatic melanoma. Neither combinations of DTIC with other chemotherapy drugs (e.g., cisplatin, vinblastine, and carmustine) nor adding interferon-α2b to DTIC have shown a survival advantage over DTIC treatment alone. Most recently, clinical trials with antibodies and vaccines to treat metastatic melanoma also failed to demonstrate satisfactory efficacy.

With the rapidly rising incidence of this disease and the high resistance to current therapeutic agents, developing more effective drugs for advanced melanoma and other cancer types that can effectively circumvent multidrug-resistance (MDR) will provide significant benefits to cancer patients.

Microtubules are cytoskeletal filaments consisting of αβ-tubulin heterodimers and are involved in a wide range of cellular functions, including shape maintenance, vesicle transport, cell motility, and division. Tubulin is the major structural component of the microtubules and a well verified target for a variety of highly successful anti-cancer drugs. Since microtubules are critically important components in cell mitosis and cell signaling, compounds that are able to interfere with microtubule-tubulin equilibrium in cells are effective in the treatment of cancers. Tubulin binding agents alter the dynamic behaviors of microtubules and arrest mitotic cells in the M-phase of the cell cycle, thus leading to apoptotic cell death. The potency, efficacy and widespread clinical use of these agents in a variety of cancers, e.g., breast, ovarian, prostate, lung, leukemias and lymphomas, stand testament to the importance of tubulin and its role in cancer growth. Anticancer drugs like taxol and vinblastine that are able to interfere with microtubule-tubulin equilibrium in cells are extensively used in cancer chemotherapy.

Despite the clinical success of tubulin inhibitors, agents currently used for chemotherapy are known to have limitations such as high peripheral neurotoxicity, low bioavailability, poor solubility, complicated synthesis procedures, and drug resistance due to P-glycoprotein- or ATP binding cassette (ABC) transporter protein-mediated drug efflux, multidrug resistance (MDR) transporters and tubulin mutation.

There are two major groups of antimitotic agents consisting of microtubule-stabilizing agents (i.e., taxanes) and microtubule-destabilizing drugs (i.e., vinca alkaloids and colchicines). They interact physically with tubulin by binding to one of the three main binding sites: colchicine-, vinblastine-, or paclitaxel-binding site.

Both the taxanes and vinca alkaloids are widely used to treat human cancers, while no colchicine-site binders are currently approved for cancer chemotherapy yet. However, colchicine binding agents like combretastatin A-4 (CA-4) and ABT-751, are now under clinical investigation as potential new chemotherapeutic agents.

A common mechanism of multidrug resistance (MDR), namely ATP binding cassette (ABC) transporter protein-mediated drug efflux, limits the efficacy of microtubule-interacting anticancer drugs, which are currently in clinical use.

P-glycoproteins (P-gp, encoded by the MDR1 gene) are important members of the ABC superfamily. P-gp prevents the intracellular accumulation of many cancer drugs by increasing their efflux out of cancer cells, as well as contributing to hepatic, renal, or intestinal clearance pathways. Attempts to co-administer P-gp modulators or inhibitors to increase cellular availability by blocking the actions of P-gp have met with limited success.

The other major problem with taxanes, as with many biologically active natural products, is its lipophilicity and lack of solubility in aqueous systems. This leads to the use of emulsifiers like Cremophor EL and Tween 80 in clinical preparations. A number of biologic effects related to these drug formulation vehicles have been described, including acute hypersensitivity reactions and peripheral neuropathies. The peripheral neurotoxicity might result from a disruption of microtubule mediated axonal flow. Microtubule inhibitors such as taxanes and vinca alkaloids are also known to induce myelosuppression and neutropenia due to the inhibition of the proliferation of non-transformed cells such as hematopoietic precursor cells.

These limitations have led to the search for new agents that inhibit tubulin activity and circumvent these limitations.

Compared to compounds binding the paclitaxel- or vinca alkaloid binding site, colchicine-binding agents usually exhibit relatively simple structures. Thus providing a better opportunity for oral bioavailability via structural optimization to improve solubility and pharmacokinetic (PK) parameters. In addition, many of these drugs appear to circumvent P-gp-mediated MDR. Therefore, these novel colchicine binding site targeted compounds hold great promise as therapeutic agents, particularly since they have improved aqueous solubility.

Naturally occurring compounds derived from both food source and non-food source plants have been tested and often have demonstrated an anticancer effect against various cancers. Derivatives and analogs of these plant compounds are constantly being isolated or synthesized to find more efficacious anticancer agents.

Recently, the compound indole-3-carbinol, a phytonutrient derived from cruciferous vegetables, such as broccoli, brussel sprouts or cabbage, has been studied as a potential anticancer therapeutic against breast, cervical, prostate, and colon cancers.

Other indole derivatives have been synthesized. U.S. Pat. No. 6,638,964 discloses indole derivatized with substituted sulfonamides useful to treat malignancies and autoimmune diseases.

U.S. Pat. No. 6,812,243 discloses highly substituted bisindoles useful as tyrosine kinase inhibitors to treat cell proliferative diseases.

However, naturally occurring or synthetic indole compounds used as anticancer agents may have drawbracks due to large dosages, loss of anticancer activity from metabolic breakdown, or toxicity. Attempts to develop effective indole derivatives that can be easily administered in reasonable doses, that retain the ability to inhibit activities associated with onset of a cell proliferative disease, and that have improved stability, increased clinical effectiveness, consistent results, and minimal toxicity and side effects are continuously ongoing.

Thus, the prior art is still deficient in the lack of indole derivatives and analogs useful as therapeutics.

### SUMMARY OF THE INVENTION

In one embodiment, this invention is directed to a compound represented by the structure of formula IV:

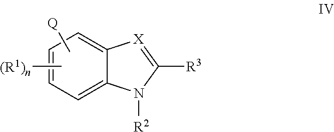

wherein:
X is CH or N;
R¹ is H, F, Cl, Br, I, CF₃, NO₂, OH, —OCH₃, CN, CH₃, alkyl, alkenyl, cycloalkyl, aryl, O-alkyl, O-aryl, or phenyl substituted at C3 or C5 with R⁴,
Q is H, F, Cl, Br, I, CF₃, NO₂, OH, —OCH₃, CN, CH₃, alkyl, alkenyl, O-alkyl, or O-aryl;
n is 0, 1, 2 or 3;
R² is H, CH₃, alkyl, benzyl, or —SO₂Ph;
R³ is phenyl substituted at C3 or C5 with R⁴; R⁸R⁹; naphthyl substituted at C5, C6, or C7 with 2-, 3- or 6-indolyl or unsubstituted, the indolyl moiety independently substituted at C1 with R², at C4, C5, or C6 with R¹ or with a combination thereof; R¹²R¹³; or 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof;
R⁴ is R⁵; C₁₋₃alkylene-R⁵; CH₂—R⁶, CH(OH)—R⁶; C(O)R⁶; CH═CH—C(R⁷)—R⁶; —C(O)—R⁷—R⁶; —O—C(R⁷)—R⁶; R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹ or R¹²R¹³;
R⁵ is OH, NO₂, NH₂, —NH—C₁₋₃alkyl, N═N═N, CN, or OR⁶;
R⁶ is H, C₁₋₃alkyl, or a 5- or 6-membered ring independently substituted at C2, C3, C4, C5, C6 or any combination thereof with R¹;
R⁷ is O, S or NH;
R⁸ is —CH₂, —CH₂OH, C═O, C═S, C═CH₂, C═NOH, C═N(NH₂);
R⁹ is H, substituted or unsubstituted indolyl, substituted or unsubstituted aryl, phenyl independently substituted at C3 with R¹⁰ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;
R¹⁰ is H, F, Cl, Br, I, CF₃, NO₂, OH, —OCH₃, CN, CH₃, alkyl, alkenyl, cycloalkyl, aryl, O-alkyl, O-aryl, substituted or unsubstituted naphthyl or forms a dioxolyl ring with R¹¹ at C4;
R¹¹ is H, OH, or —OCH₃;
R¹² is pyrrolyl, furanyl, thienyl, or cyclopentadienyl;
R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-naphthyl or —C(O)phenyl, each independently substituted with at C2, C3, C4, C5, or C6 with R¹; or its isomer, tautomer, pharmaceutically acceptable salt, pharmaceutical product, N-oxide, hydrate or any combination thereof.

In another embodiment, the compound is represented by the structure of formula II(a):

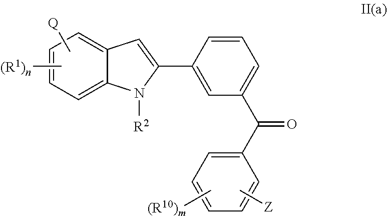

wherein:

R¹, R¹⁰, Q and Z are each independently H, F, Cl, Br, I, CF₃, NO₂, OH, —OCH₃,

CN, CH₃, alkyl, alkenyl, cycloalkyl, aryl, O-alkyl or, O-aryl;

n is 0, 1, 2 or 3;

m is 0, 1, 2, 3, or 4;

R² is H, CH₃, alkyl, benzyl or —SO₂Ph.

In another embodiment, the compound is represented by the structure of formula IV(a):

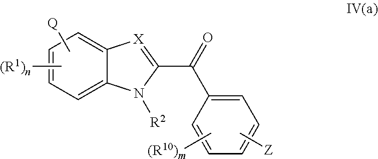

wherein X is CH or N; and R¹, R¹⁰, Q, Z, n, m and R² are as defined for compound of formula II(a) above.

In one embodiment, this invention is directed to a compound represented by the structure of formula V:

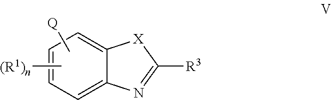

wherein X is CH₂, NH, N(R²), O, S, SO or SO₂; and R¹, R², Q, n and R³ are as defined for compound of formula IV above;
or its isomer, tautomer, pharmaceutically acceptable salt, pharmaceutical product, N-oxide, hydrate or any combination thereof.

In another embodiment, the compound is represented by the structure of formula V(a):

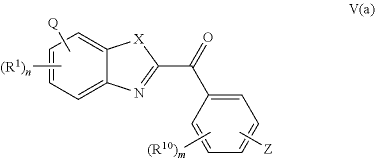

wherein X is CH₂, NH, N(R²), O, S, SO or SO₂; and R¹, R², R¹⁰, Q, Z, n and m, are as defined for compound of formula II(a) above.

In another embodiment, this invention is directed to pharmaceutical composition comprising a compound of this invention, and a pharmaceutically acceptable carrier, diluent, salt or any combination thereof.

In another embodiment, this invention is directed to a method of inhibiting tubulin polymerization in a cell associated with a cell proliferative disease. In another embodiment, the cell proliferative disease is a cancer. In another embodiment, the cancer is prostate cancer, melanoma, colon cancer, bladder cancer or breast cancer.

In another embodiment, this invention is directed to a method of treating, halting, suppressing, reducing the severity, reducing the incidence of, reducing the risk, causing the regression of, or inhibiting prostate cancer in a subject, comprising the step of administering to a subject an effective amount of a compound according to this invention. In another embodiment, the prostate cancer is drug resistant prostate cancer, multidrug-resistant (MDR) prostate cancer, castration-resistant prostate cancer, metastatic prostate cancer, advanced prostate cancer or any combination thereof.

In another embodiment, this invention is directed to a method of treating, halting, suppressing, reducing the severity, reducing the incidence of, reducing the risk, causing the regression of, or inhibiting melanoma in a subject, comprising the step of administering to a subject an effective amount of a compound according to this invention. In another embodiment, the melanoma is drug resistant melanoma, multidrug-resistant (MDR) melanoma, metastatic melanoma, or any combination thereof.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1J depict representative synthetic schemes and representative structures for compounds according to the present invention. Synthetic schemes are shown for compounds 10, 11 and 13 in FIG. 1A. Structures for compounds 14-31 are shown in FIG. 1B. Synthetic schemes for preparing compounds of this invention are shown in FIGS. 1C-1J.

FIG. 2 depicts synthetic scheme for compounds 66-84. Reagents and conditions: a) Arylboronic acid (1.2 equiv.), Pd(PPh₃)₄ (0.04 equiv.), K₂CO₃, H₂O, DMF, 5 h; b) i. BuLi (1.2 equiv.), THF, −78° C. 1 h; ii. 5 (0.9 equiv.), −78° C. rt overnight or ArMgBr, THF, rt 3 h; c) PDC (6 equiv.), DMF, rt, 16 h; d) NaOH, H₂O, EtOH, reflux overnight; e) Pd/C, EtOH, 2 atm H₂, 24 h.

FIG. 3 depicts synthetic scheme for compound 90. Reagents and conditions: (a) i. LDA, −78° C., 1 h; ii. BrCN, −78 to 25° C., 16 h; (b) 3-formylphenyl boronic acid, Pd(PPh₃)₄, K₂CO₃, DMF, Reflux, 3-16 h; (c) 3,4,5-trimethoxyphenylithium, THF, −78 to 25° C., 16 h; (d) PDC, DMF, 16 h or Dess-Martin Periodinate, CH₂Cl₂, 2 h; (e) KOH, H2O, EtOH, reflux, 16 h.

FIG. 4 depicts synthetic scheme for compounds 98-123 and 146-148. Reagents and conditions: (a) NaH, PhSO₂Cl, THF, 0° C.-rt; (b) t-BuLi (1.7 M in pentane), benzoyl chloride, THF, −78° C.; (c) NaOH, ethanol, H₂O, reflux; (d) NaH, CH₃I for 119 and 121 or BnBr for 120, THF, reflux.

FIG. 5 depicts the chemical structure of 68 and apoptosis induced by 68 treatment. A, Chemical structures of 68, vinblastine, and docetaxel. B, 68 induces DNA fragmentation in PC-3 cells. The extent of apoptosis (i.e., the enrichment factor) was determined using a commercially available anti-histone ELISA after 24 h treatment. C, 68 induced the Bcl-2 phosphorylation in PC-3 cells (24 h) and actin was used as a loading control.

FIG. 6 depicts the effect of 68 on cell cycle. PC-3 cells were treated with different concentrations (0 to 1 μM) of 68, vinblastine, or docetaxel for 24 h, and DNA content of the cells was analyzed by FACS. A, 68 arrested cells in G₂M phase. B, The percentage of cells in G₂M phase of the cell division cycle was quantified and dose response curves are shown. C, Changes in expressed and phosphorylated status of G₂-M regulators including cyclin B1, Cdc25C, pCdc25C, Cdc2 and pCdc2 by 68 were evaluated in PC-3 cells (24 h). Actin was used as a loading control and relative band intensity was shown as the mean±SD (n=2-3). * P<0.05; # P<0.01.

FIG. 7 depicts the inhibition of microtubule formation by 68 and other agents. A, PC-3 cells were treated with antimitotic agents for 24 h. Fixed cells were incubated with anti-α-tubulin-FITC antibody and the cellular microtubules were observed with a Zeiss Axioplan 2 fluorescent microscope. B, the microtubule polymerization was monitored by measuring the turbidity at 340 nm in the absence or presence of drugs. Representative experiment. Control (▪); 68, 0.1 μM (); 68, 1 μM (◯); 68, 5 μM (▾); 68, 10 μM (Δ). Data shown are the mean of duplicate reactions. The percentage of microtubule polymerization at 40 min was quantified and dose response curves are shown (vehicle control set at 100%). C, [³H]Vinblastine was incubated with tubulin in the presence of different concentrations of vincristine or 68. [³H]Podophyllotoxin was incubated with tubulin in the presence of different concentrations of colchicine or 68. Tubulin-bound [³H]podophylloxin and [³H]vinblastine were plotted against the concentrations of the competitors.

FIG. 8 depicts the efficacy and tolerability of 68 in xenograft models after i.p. injection. A, PC-3 xenografts were treated with vehicle (2 days/week), docetaxel (5 mg/kg, 2 days/week), or 68 (5 and 10 mg/kg, 2 days/week) for 4 weeks. B, Xenograft models using MES-SA cells were treated with vehicle (q2d), vinblastine (0.5 mg/kg, q2d), docetaxel (5 mg/kg, q2d), or 68 (10 mg/kg, q2d). C. Xenograft models bearing MES-SA/DX5 cells over-expressing P-gp were treated as same as the MES-SA xenograft model. The tumor volumes (mm³) were plotted against time and are the means±SD from eight animals. The tumor volumes were shown in left panel and survival rates or body weights were shown in right panel.

FIG. 9 depicts the in vivo and in vitro neurotoxicity of 68. A, PC-12 cells pretreated with 100 ng/mL murine 2.5S β-NGF were exposed to vehicle control, vinblastine, or 68 at various concentrations in the presence of 100 ng/mL NGF for 24 h. The numbers of cells with no neurites, short neurites (<2×cell body), and long neurites (>2×cell body) were counted (left panel). All determinations were confirmed using three independent experiments. PC-12 cell survival was quantitated by the SRB assay (right panel). Each value represents the mean±SD of three independent experiments. B, ICR mice were dosed with vehicle control, vinblastine (0.5 mg/kg, i.p., q2d), vincristine (0.5 mg/kg, i.p., q2d), or 68 (10 mg/kg, i.p., q2d) for 2 weeks. Mice were placed on an accelerating rotating rod and the performance on the rotarod was monitored 2 days/week (n=10). * P<0.02; # P<0.01.

FIGS. 10A-10C illustrate that compound 13 induces apoptosis (FIG. 10A), decreases anti-apoptosis proteins (FIG. 10B) and induces DNA fragmentation (FIG. 10C) in LNCaP and PC-3 cells.

FIGS. 11A-11B illustrate that compound 13 induces G2/M phase arrest (FIG. 11A) in LNCaP cells and inhibits polymerization of tubulin proteins in vitro (FIG. 11B).

FIG. 12 illustrates the effect of 50, 100 and 200 mg/kg of compound 13 on body weight of ICR mice.

FIG. 13 illustrates the mean plasma concentration-time profile of compound 13 in mice.

FIG. 14 illustrates the antitumor activity of compound 13 against a PC-3 xenograft in Balb/c mice.

### DETAILED DESCRIPTION OF EMBODIMENTS

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

As used herein, the term “alkyl” refers to in one embodiment, to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. Non limiting examples of alkyl are methyl, ethyl, propyl, isopropyl, butyl and tert-butyl. In one embodiment, the alkyl group is methyl (CH₃). The alkyl group may be unsubstituted or substituted by one or more groups selected from halogen, hydroxy, alkoxy, carbonyl, amido, alkylamido, dialkylamido, nitro, amino, alkylamino, dialkylamino, carboxyl, thio and thioalkyl.

An “alkenyl” group refers, in another embodiment, to an unsaturated hydrocarbon, including straight chain, branched chain and cyclic groups having one or more double bond. The alkenyl group may have one double bond, two double bonds, three double bonds, etc. Examples of alkenyl groups are ethenyl, propenyl, butenyl, cyclohexenyl, etc. In one embodiment, the alkylene group has 1-12 carbons. In another embodiment, the alkylene group has 1-7 carbons. In another embodiment, the alkylene group has 1-6 carbons. In another embodiment, the alkylene group has 1-4 carbons. The alkenyl group may be unsubstituted or substituted by one or more groups selected from halogen, hydroxy, alkoxy, carbonyl, amido, alkylamido, dialkylamido, nitro, amino, alkylamino, dialkylamino, carboxyl, thio and thioalkyl.

A “haloalkyl” group refers to an alkyl group as defined above, which is substituted by one or more halogen atoms, in one embodiment by F, in another embodiment by Cl, in another embodiment by Br, in another embodiment by I.

A “cycloalkyl” group refers to a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are selected from halogen, haloalkyl, hydroxy, alkoxy, carbonyl, amido, alkylamido, dialkylamido, nitro, amino, alkylamino, dialkylamino, carboxy, thio or thioalkyl. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalin, norbornyl, adamantyl and the like.

An “aryl” group refers to an aromatic group having at least one carbocyclic aromatic group or heterocyclic aromatic group, which may be unsubstituted or substituted by one or more groups selected from halogen, haloalkyl, alkyl, alkenyl, aryl, hydroxy, alkoxy, aryloxy, carbonyl, amido, alkylamido, dialkylamido, nitro, cyano, amino, alkylamino, dialkylamino, carboxy, thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like. In one embodiment, the aryl group is a 4-8 membered ring. In another embodiment, the aryl group is a 4-12 membered ring(s). In another embodiment, the aryl group is a 3-10 membered ring(s). In another embodiment, the aryl group is a 3-8 membered ring(s). In another embodiment, the aryl group is a 6 membered ring. In another embodiment, the aryl group is a 5 membered ring. In another embodiment, the aryl group is 2-4 fused ring system. “Heteroaryl” refers to an aryl compound with one or more heteroatoms, e.g., nitrogen, sulfur or oxygen, in the aromatic ring structure.

A “hydroxyl” group refers to an OH group.

The term “halogen” or “halide” refers to in one embodiment to F, in another embodiment to Cl, in another embodiment to Br, in another embodiment to I.

Numbering of the carbon atoms uses standard protocol where the nitrogen heteroatom in indole is C1 and the carbon atom in the phenyl moiety linked to C2 in indole is C1. This numbering protocol also is used with any substituent ring structure comprising these indole or diindole derivatives or analogs, such as, a cyclic alkyl, an aryl or a heteroaryl moiety.

In one embodiment, the numbering of carbon atoms in indole and its derivatives is represented by the following scheme:

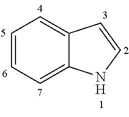

In one embodiment, the numbering of carbon atoms in benzimidazole and its derivatives is represented by the following scheme:

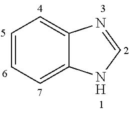

In one embodiment, the numbering of carbon atoms in benzoxazole and its derivatives is represented by the following scheme:

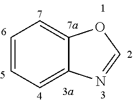

In one embodiment, the numbering of carbon atoms in benzothiazole and its derivatives is represented by the following scheme:

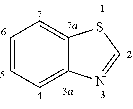

As used herein, the term “contacting” refers to any suitable method of bringing an inhibitory agent into contact with a cell. In some examples the cell is an abnormally proliferating cell. In vitro or ex vivo this is achieved by exposing the cells to the inhibitory agent in a suitable medium. For in vivo applications, any known method of administration is suitable.

As used herein, the term “treating” or the phrase “treating a cancer” includes, but is not limited to, halting the growth of cancer cells, killing the cancer cells or a mass comprising the same, or reducing the number of cancer cells or the size of a mass comprising the same. Halting the growth refers to halting any increase in the size or the number of cancer cells or in a mass comprising the same or to halting the division of the cancer cells. Reducing the size refers to reducing the size of a mass comprising the cancer cells or the number of or size of the same cells. As would be apparent to one of ordinary skill in the art, the term “cancer” or “cancer cells” or “tumor” refers to examples of neoplastic cell proliferative diseases and refers to a mass of malignant neoplastic cells or a malignant tissue comprising the same.

As used herein, the term “inhibiting” or “inhibition” of tubulin polymerization in cells associated with a cell proliferative disease, e.g., cells comprising a cancer or tumor or malignant or abnormally proliferating cells, shall include partial or total inhibition of tubulin formation and also is meant to include decreases in the rate of proliferation or growth of the cells associated with the cell proliferative disease. The biologically inhibitory dose of the composition of the present invention may be determined by assessing the effects of the test element on tubulin polymerization in target malignant or abnormally proliferating cells in tissue culture or cell culture, on tumor growth in animals or any other method known to those of ordinary skill in the art.

As used herein, the term “subject” refers to any target of the treatment.

This invention provides, in one embodiment, a compound characterized by the structure of Formula I-V(a). In one embodiment, the compound is useful in treating cancer, including, inter alia, prostate cancer, drug resistant prostate cancer, castration-resistant prostate cancer, metastatic prostate cancer, advanced prostate cancer, melanoma, drug resistant melanoma, metastatic melanoma, breast cancer, drug resistant breast cancer, metastatic breast cancer, colon cancer and bladder cancer.

In some embodiments, this invention provides synthetic processes of preparation of the compounds of this invention. In some embodiments, the invention provides compositions comprising the compounds or use of the same.

In one embodiment, the present invention provides an indole derivative compound represented by the structure of formula I:

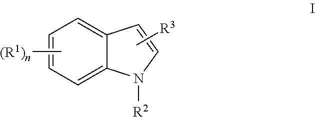

wherein:

R¹ is H, F, Cl, Br, I, CF₃, NO₂, OH, —OCH₃, CN, CH₃, alkyl, alkenyl, cycloalkyl, aryl, O-alkyl, O-aryl, or phenyl substituted at C3 or C5 with R⁴,

n is 0, 1, 2 or 3;

R² is H, CH₃, alkyl, benzyl or —SO₂Ph;

R³ is H, F, Cl, Br, I, phenyl substituted at C3 or C5 with R⁴; R⁸R⁹; naphthyl substituted at C5, C6, or C7 with 2-, 3- or 6-indolyl or unsubstituted, the indolyl moiety independently substituted at C1 with R², at C4, C5, or C6 with R¹ or with a combination thereof; R¹²R¹³; or 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof;

R⁴ is R⁵; C₁₋₃alkylene-R⁵; CH₂—R⁶, CH(OH)—R⁶; C(O)R⁶; CH═CH—C(R⁷)—R⁶; —C(O)—R⁷—R⁶; —O—C(R⁷)—R⁶; R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹ or R¹²R¹³;

R⁵ is OH, NO₂, NH₂, —NH—C₁₋₃alkyl, N═N═N, CN, or OR⁶;

R⁶ is H, C₁₋₃alkyl, or a 5- or 6-membered ring independently substituted at C2, C3, C4, C5, C6 or any combination thereof with R¹;

R⁷ is O, S or NH;

R⁸ is —CH₂, —CH(OH), C═O, C═S, C═CH₂, C═NOH or C═N(NH₂);

R⁹ is H, substituted or unsubstituted indolyl, substituted or unsubstituted aryl, phenyl independently substituted at C3 with R¹⁰ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R¹⁰ is H, F, Cl, Br, I, CF₃, NO₂, OH, —OCH₃, CN, CH₃, alkyl, alkenyl, cycloalkyl, aryl, O-alkyl, O-aryl, substituted or unsubstituted naphthyl or forms a dioxolyl ring with R¹¹ at C4;

R¹¹ is H, OH, or —OCH₃;

R¹² is pyrrolyl, furanyl, thienyl, or cyclopentadienyl;

R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-naphthyl or —C(O)phenyl, each independently substituted with at C2, C3, C4, C5, or C6 with R¹;

or its isomer, tautomer, pharmaceutically acceptable salt, pharmaceutical product, N-oxide, hydrate or any combination thereof.

In one embodiment, R¹ of compound of formula I is H, R³ is a phenyl substituted at C3 or C5 with R⁴, and R⁴ is R⁸R⁹.

In another embodiment, compound of formula I is represented by the structure:

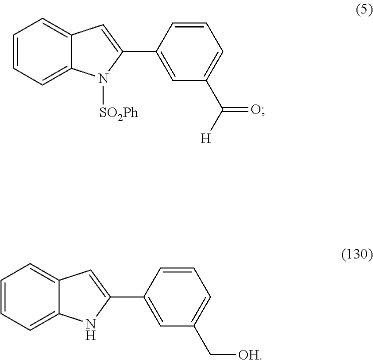

In another embodiment, R¹ is H or F, R³ is a phenyl substituted at C3 or C5 with R⁴, and R⁴ is —R⁸-(2- or 3-indolyl).

In another embodiment, compound of formula I is represented by the structure:

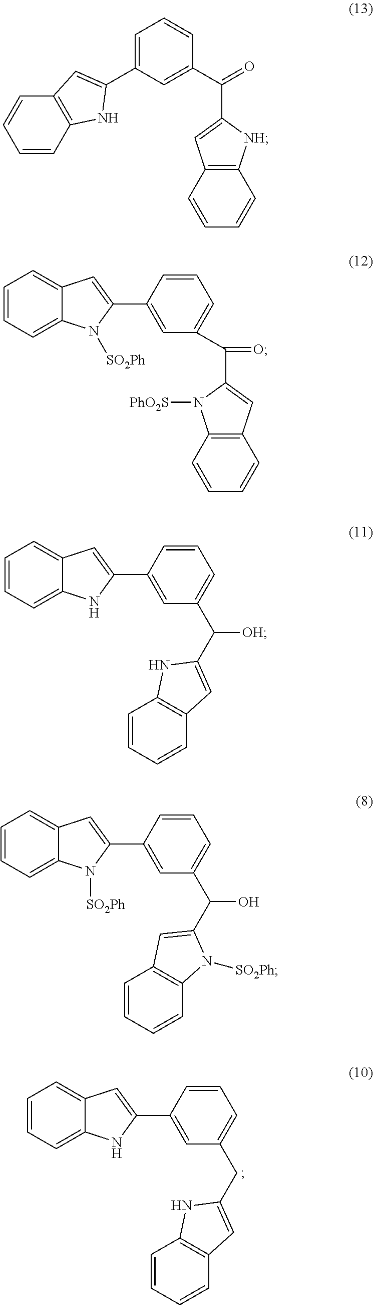

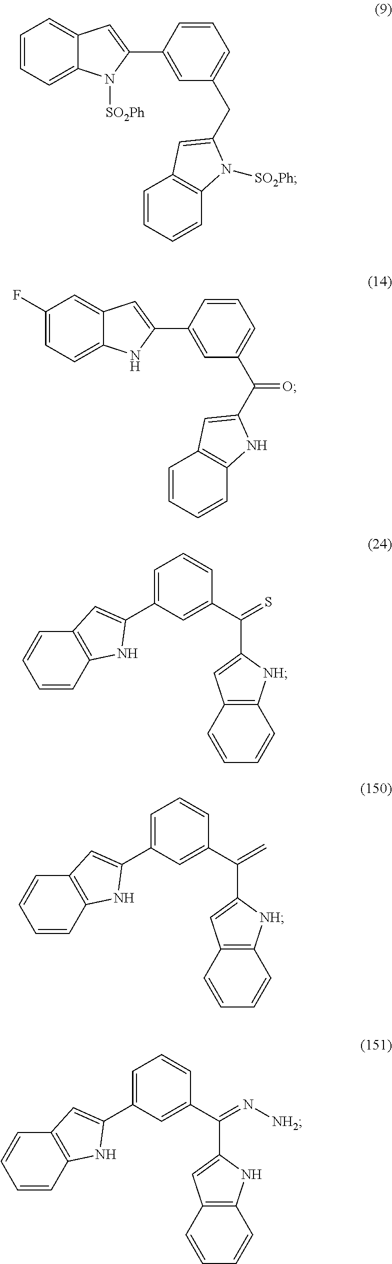

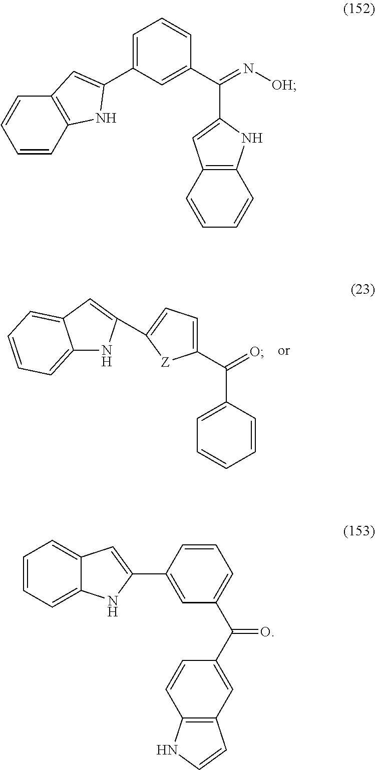

In yet another embodiment, R³ is a phenyl substituted at C3 or C5 with R⁴ and R⁴ is R⁷R⁸-(2-, 3-, or 6-indolyl).

In another embodiment, compound of formula I is represented by the structure:

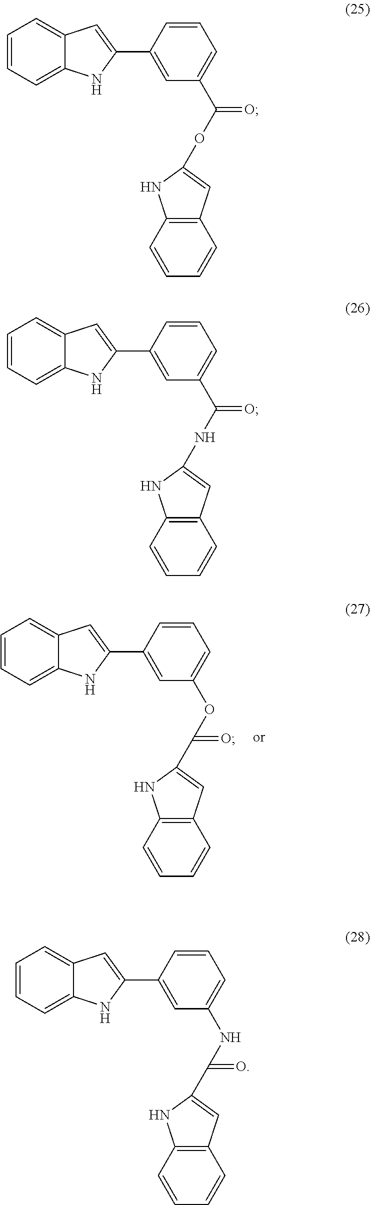

In another embodiment, R³ is a phenyl substituted at C3 or C5 with R⁴ and R⁴ is R⁸R⁹.

In another embodiment, compound of formula I is represented by the structure:

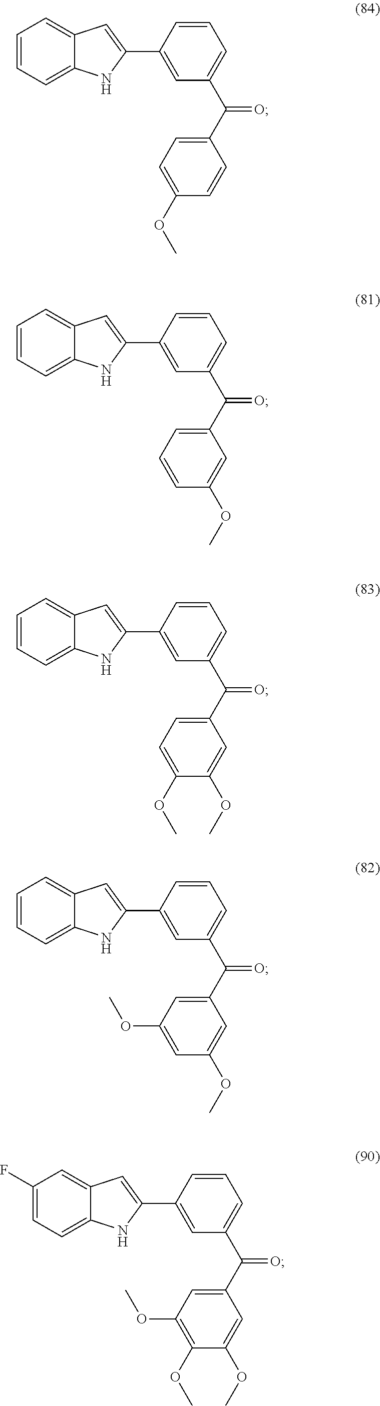

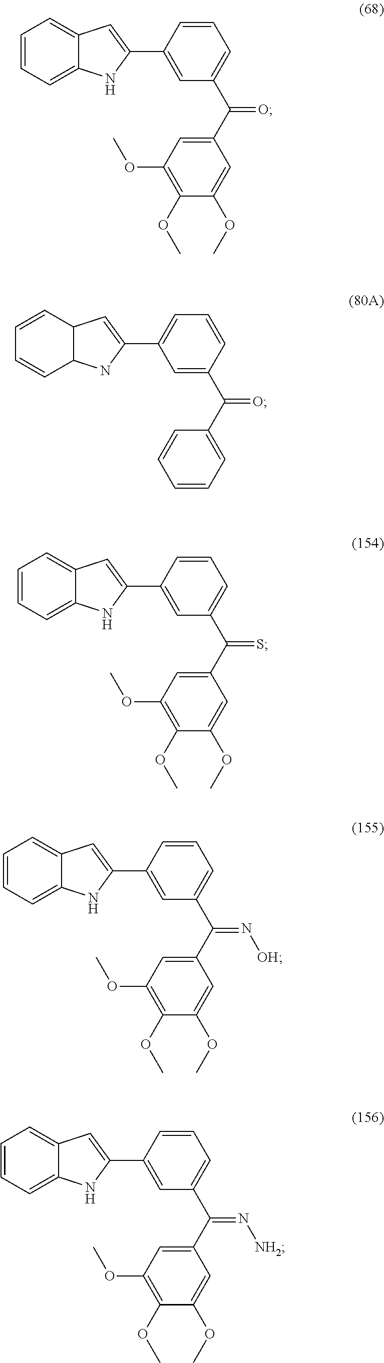

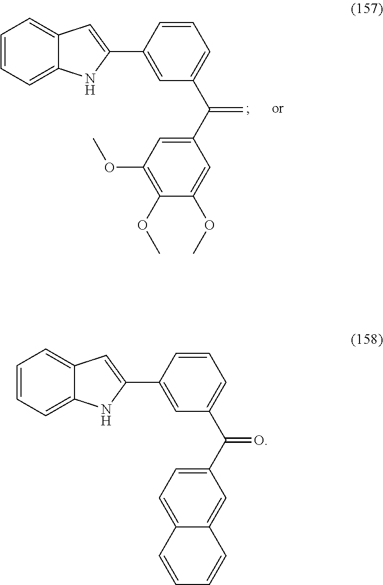

In another embodiment, compound of formula I is represented by the structure:

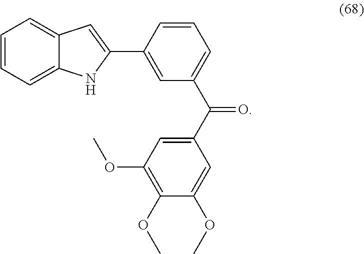

In another embodiment, R³ of formula I is 2-, 3- or 6-indolyl.

In another embodiment, compound of formula I is represented by the structure:

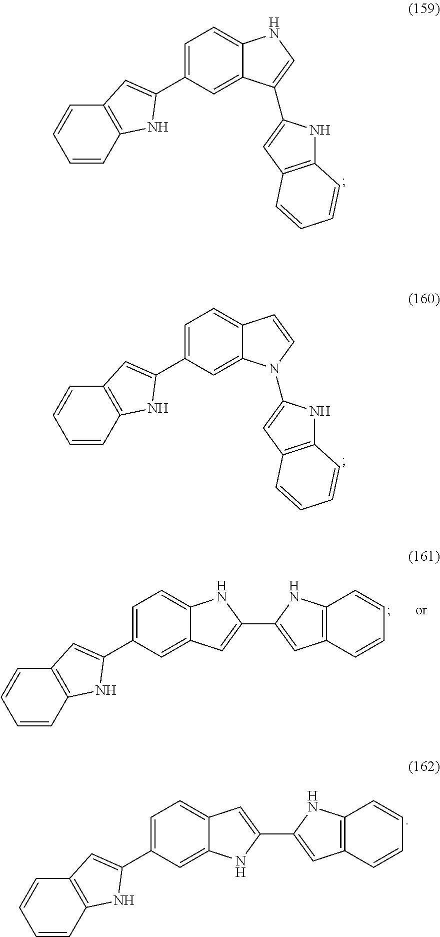

In yet another embodiment, R³ of a compound of formula I is naphthyl.

In another embodiment, compound of formula I is represented by the structure:

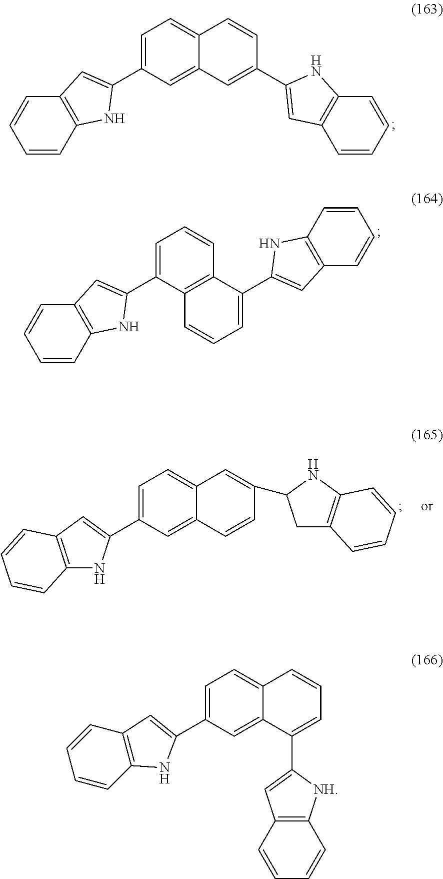

In yet another embodiment, R³ of a compound of formula I is R⁸R⁹.

In another embodiment, compound of formula I is represented by the structure:

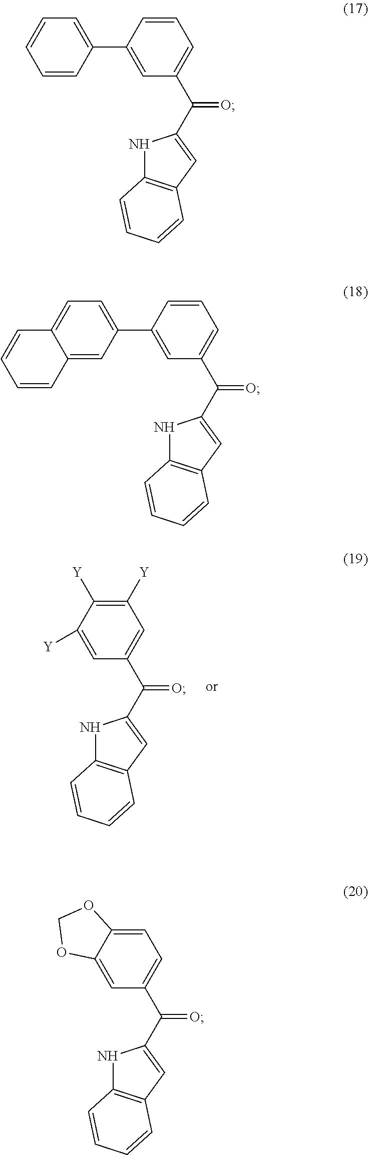

wherein Y is independently selected from H, OH, OCH₃.

In yet another embodiment, R³ of a compound of formula I is R¹²R¹³.

In another embodiment, compound of formula I is represented by the structure:

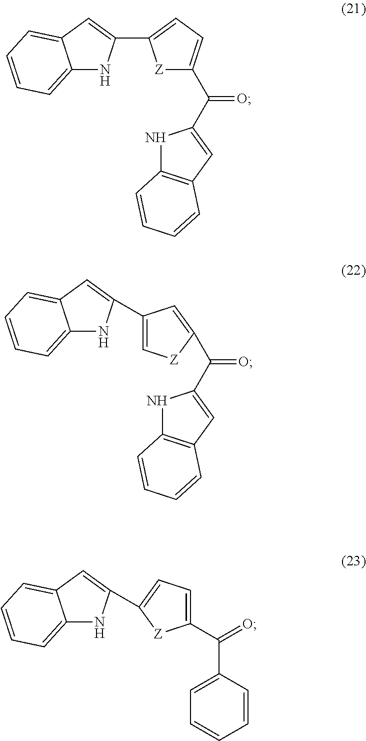

wherein Z is independently selected from S, O, NH, and CH₂.

In one embodiment, the present invention provides an indole derivative compound represented by the structure of formula II:

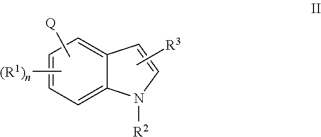

wherein:

R¹ is H, F, Cl, Br, I, CF₃, NO₂, OH, —OCH₃, CN, CH₃, alkyl, alkenyl, cycloalkyl, aryl, O-alkyl, O-aryl, or phenyl substituted at C3 or C5 with R⁴,

Q is H, F, Cl, Br, I, CF₃, NO₂, OH, —OCH₃, CN, alkyl, alkenyl, aryl, O-alkyl or O-aryl;

n is 0, 1, 2 or 3;

R² is H, CH₃, alkyl, benzyl or —SO₂Ph;

R³ is H, F, Cl, Br, I, phenyl substituted at C3 or C5 with R⁴; R⁸R⁹; naphthyl substituted at C5, C6, or C7 with 2-, 3- or 6-indolyl or unsubstituted, the indolyl moiety independently substituted at C1 with R², at C4, C5, or C6 with R¹ or with a combination thereof; R¹²R¹³; or 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof;

R⁴ is R⁵; C₁₋₃alkylene-R⁵; CH₂—R⁶, CH(OH)—R⁶; C(O)R⁶; CH═CH—C(R⁷)—R⁶; —C(O)—R⁷—R⁶; —O—C(R⁷)—R⁶; R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹ or R¹²R¹³;

R⁵ is OH, NO₂, NH₂, —NH—C₁₋₃alkyl, N═N═N, CN, or OR⁶;

R⁶ is H, C₁₋₃alkyl, or a 5- or 6-membered ring independently substituted at C2, C3, C4, C5, C6 or any combination thereof with R¹;

R⁷ is O, S or NH;

R⁸ is —CH₂, —CH(OH), C═O, C═S, C═CH₂, C═NOH, C═N(NH₂);

R⁹ is H, substituted or unsubstituted indolyl, substituted or unsubstituted aryl; phenyl independently substituted at C3 with R¹⁰ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R¹⁰ is H, F, Cl, Br, I, CF₃, NO₂, OH, —OCH₃, CN, CH₃, alkyl, alkenyl, cycloalkyl, aryl, O-alkyl, O-aryl, substituted or unsubstituted naphthyl or forms a dioxolyl ring with R¹¹ at C4;

R¹¹ is H, OH, or —OCH₃;

R¹² is pyrrolyl, furanyl, thienyl, or cyclopentadienyl;

R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-naphthyl or —C(O)phenyl, each independently substituted with at C2, C3, C4, C5, or C6 with R¹; or its isomer, tautomer, pharmaceutically acceptable salt, pharmaceutical product, N-oxide, hydrate or any combination thereof.

In one embodiment, the present invention provides an indole derivative compound represented by the structure of formula II(a):

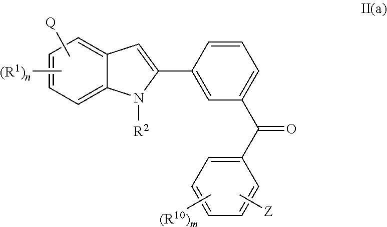

wherein:

R¹, R¹⁰, Q and Z are each independently H, F, Cl, Br, I, CF₃, NO₂, OH, —OCH₃, CN, CH₃, alkyl, alkenyl, cycloalkyl, aryl, O-alkyl or, O-aryl;

n is 0, 1, 2 or 3;

m is 0, 1, 2, 3, or 4;

R² is H, CH₃, alkyl, benzyl or —SO₂Ph;

or its isomer, tautomer, pharmaceutically acceptable salt, pharmaceutical product, N-oxide, hydrate or any combination thereof.

In one embodiment, the present invention provides a compound represented by the structure of formula III:

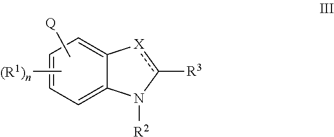

wherein:

X is CH, CH₂, N, NH, O, S or SO₂;

R¹ is H, F, Cl, Br, I, CF₃, NO₂, OH, —OCH₃, CN, CH₃, alkyl, alkenyl, cycloalkyl, aryl, O-alkyl, O-aryl, or phenyl substituted at C3 or C5 with R⁴,

Q is H, F, Cl, Br, I, CF₃, NO₂, OH, —OCH₃, or CN, CH₃, alkyl, alkenyl, O-alkyl or O-aryl;

n is 0, 1, 2 or 3;

R² is H, CH₃, alkyl, benzyl, or —SO₂Ph;

R³ is H, F, Cl, Br, I, phenyl substituted at C3 or C5 with R⁴; R⁸R⁹; naphthyl substituted at C5, C6, or C7 with 2-, 3- or 6-indolyl or unsubstituted, the indolyl moiety independently substituted at C1 with R², at C4, C5, or C6 with R¹ or with a combination thereof; R¹²R¹³; or 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof;

R⁴ is R⁵; C₁₋₃alkylene-R⁵; CH₂—R⁶, CH(OH)—R⁶; C(O)R⁶; CH═CH—C(R⁷)—R⁶; —C(O)—R⁷—R⁶; —O—C(R⁷)—R⁶; R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹ or R¹²R¹³;

R⁵ is OH, NO₂, NH₂, —NH—C₁₋₃alkyl, N═N═N, CN, or OR⁶;

R⁶ is H, C₁₋₃alkyl, or a 5- or 6-membered ring independently substituted at C2, C3, C4, C5, C6 or any combination thereof with R¹;

R⁷ is O, S or NH;

R⁸ is —CH₂, —CH(OH), C═O, C═S, C═CH₂, C═NOH, C═N(NH₂);

R⁹ is H, substituted or unsubstituted indolyl, substituted or unsubstituted aryl; phenyl independently substituted at C3 with R¹³ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R¹³ is H, F, Cl, Br, I, CF₃, NO₂, OH, —OCH₃, CN, CH₃, alkyl, alkenyl, cycloalkyl, aryl, O-alkyl, O-aryl, substituted or unsubstituted naphthyl or forms a dioxolyl ring with R¹¹ at C4;

R¹¹ is H, OH, or —OCH₃;

R¹² is pyrrolyl, furanyl, thienyl, or cyclopentadienyl;

R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-naphthyl or —C(O)phenyl, each independently substituted with at C2, C3, C4, C5, or C6 with R¹;

or its isomer, tautomer, pharmaceutically acceptable salt, pharmaceutical product, N-oxide, hydrate or any combination thereof.

In one embodiment, the present invention provides a compound represented by the structure of formula IV:

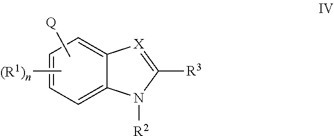

wherein:

X is CH or N;

R¹ is H, F, Cl, Br, I, CF₃, NO₂, OH, —OCH₃, CN, CH₃, alkyl, alkenyl, cycloalkyl, aryl, O-alkyl, O-aryl, or phenyl substituted at C3 or C5 with R⁴,

Q is H, F, Cl, Br, I, CF₃, NO₂, OH, —OCH₃, CN, CH₃, alkyl, alkenyl, O-alkyl, or O-aryl;

n is 0, 1, 2 or 3;

R² is H, CH₃, alkyl, benzyl, or —SO₂Ph;

R³ is H, F, Cl, Br, I, phenyl substituted at C3 or C5 with R⁴; R⁸R⁹; naphthyl substituted at C5, C6, or C7 with 2-, 3- or 6-indolyl or unsubstituted, the indolyl moiety independently substituted at C1 with R², at C4, C5, or C6 with R¹ or with a combination thereof; R¹²R¹³; or 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof;

R⁴ is R⁵; C₁₋₃alkylene-R⁵; CH₂—R⁶, CH(OH)—R⁶; C(O)R⁶; CH═CH—C(R⁷)—R⁶; —C(O)—R⁷—R⁶; —O—C(R⁷)—R⁶; R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹ or R¹²R¹³;

R⁵ is OH, NO₂, NH₂, —NH—C₁₋₃alkyl, N═N═N, CN, or OR⁶;

R⁶ is H, C₁₋₃alkyl, or a 5- or 6-membered ring independently substituted at C2, C3, C4, C5, C6 or any combination thereof with R¹;

R⁷ is O, S or NH;

R⁸ is —CH₂, —CH(OH), C═O, C═S, C═CH₂, C═NOH, C═N(NH₂);

R⁹ is H, substituted or unsubstituted indolyl, substituted or unsubstituted aryl, phenyl independently substituted at C3 with R¹⁰ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R¹⁰ is H, F, Cl, Br, I, CF₃, NO₂, OH, —OCH₃, CN, CH₃, alkyl, alkenyl, cycloalkyl, aryl, O-alkyl, O-aryl, substituted or unsubstituted naphthyl or forms a dioxolyl ring with R¹¹ at C4;

R¹¹ is H, OH, or —OCH₃;

R¹² is pyrrolyl, furanyl, thienyl, or cyclopentadienyl;

R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-naphthyl or —C(O)phenyl, each independently substituted with at C2, C3, C4, C5, or C6 with R¹;

or its isomer, tautomer, pharmaceutically acceptable salt, pharmaceutical product, N-oxide, hydrate or any combination thereof.

In one embodiment, the present invention provides a compound represented by the structure of formula IV(a):

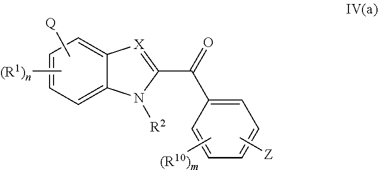

wherein:

X is CH or N;

R¹, R¹⁰, Q and Z are each independently H, F, Cl, Br, I, CF₃, NO₂, OH, —OCH₃, CN, CH₃, alkyl, alkenyl, cycloalkyl, aryl, O-alkyl or, O-aryl;

n is 0, 1, 2 or 3;

m is 0, 1, 2, 3, or 4;

R² is H, CH₃, alkyl, benzyl or —SO₂Ph; or its isomer, tautomer, pharmaceutically acceptable salt, pharmaceutical product, N-oxide, hydrate or any combination thereof.

In one embodiment, the present invention provides a compound represented by the structure of formula V:

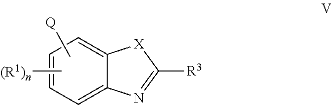

wherein:

X is CH₂, NH, N(R²), O, S, SO or SO₂;

R¹ is H, F, Cl, Br, I, CF₃, NO₂, OH, —OCH₃, CN, CH₃, alkyl, alkenyl, cycloalkyl, aryl, O-alkyl, O-aryl, or phenyl substituted at C3 or C5 with R⁴,

Q is H, F, Cl, Br, I, CF₃, NO₂, OH, —OCH₃, CN, CH₃, alkyl, alkenyl, O-alkyl, or O-aryl;

n is 0, 1, 2 or 3;

R² is H, CH₃, alkyl, benzyl, or —SO₂Ph;

R³ is H, F, Cl, Br, I, phenyl substituted at C3 or C5 with R⁴; R⁸R⁹; naphthyl substituted at C5, C6, or C7 with 2-, 3- or 6-indolyl or unsubstituted, the indolyl moiety independently substituted at C1 with R², at C4, C5, or C6 with R¹ or with a combination thereof; R¹²R¹³; or 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof;

R⁴ is R⁵; C₁₋₃alkylene-R⁵; CH₂—R⁶, CH(OH)—R⁶; C(O)R⁶; CH═CH—C(R⁷)—R⁶; —C(O)—R⁷—R⁶; —O—C(R⁷)—R⁶; R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹ or R¹²R¹³;

R⁵ is OH, NO₂, NH₂, —NH—C₁₋₃alkyl, N═N═N, CN, or OR⁶;

R⁶ is H, C₁₋₃alkyl, or a 5- or 6-membered ring independently substituted at C2, C3, C4, C5, C6 or any combination thereof with R¹;

R⁷ is O, S or NH;

R⁸ is —CH₂, —CH(OH), C═O, C═S, C═CH₂, C═NOH, C═N(NH₂);

R⁹ is H, substituted or unsubstituted indolyl, substituted or unsubstituted aryl, phenyl independently substituted at C3 with R¹³ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;

R¹⁰ is H, F, Cl, Br, I, CF₃, NO₂, OH, —OCH₃, CN, CH₃, alkyl, alkenyl, cycloalkyl, aryl, O-alkyl, O-aryl, substituted or unsubstituted naphthyl or forms a dioxolyl ring with R¹¹ at C4;

R¹¹ is H, OH, or —OCH₃;

R¹² is pyrrolyl, furanyl, thienyl, or cyclopentadienyl;

R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-naphthyl or —C(O)phenyl, each independently substituted with at C2, C3, C4, C5, or C6 with R¹; or its isomer, tautomer, pharmaceutically acceptable salt, pharmaceutical product, N-oxide, hydrate or any combination thereof.

In one embodiment, the present invention provides a compound represented by the structure of formula V(a):

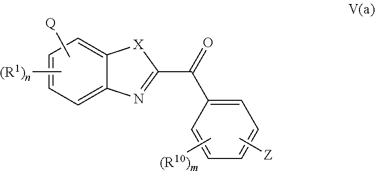

wherein:

X is CH₂, NH, N(R²), O, S, SO or SO₂;

R¹, R¹⁰, Q and Z are each independently H, F, Cl, Br, I, CF₃, NO₂, OH, —OCH₃, CN, CH₃, alkyl, alkenyl, cycloalkyl, aryl, O-alkyl or, O-aryl;

R² is H, CH₃, alkyl, benzyl, or —SO₂Ph;

n is 0, 1, 2 or 3;

m is 0, 1, 2, 3, or 4;

or its isomer, tautomer, pharmaceutically acceptable salt, pharmaceutical product, N-oxide, hydrate or any combination thereof.

In one embodiment X of compound of formula III is CH. In another embodiment, X of compound of formula III is N. In another embodiment, X of compound of formula III is NH. In another embodiment, X of compound of formula III is O. In another embodiment, X of compound of formula III is S. In another embodiment, X of compound of formula III is SO₂. In another embodiment, X of compound of formula III is CH₂.

In one embodiment X of compound of formula IV or IV(a) is CH. In another embodiment, X of compound of formula IV or IV(a) is N.

In one embodiment X of compound of formula V or V(a) is NH. In another embodiment, X of compound of formula V or V(a) is N(R²). In another embodiment, X of compound of formula V or V(a) is O. In another embodiment, X of compound of formula V or V(a) is S. In another embodiment, X of compound of formula V or V(a) is SO. In another embodiment, X of compound of formula V or V(a) is SO₂. In another embodiment, X of compound of formula V or V(a) is CH₂.

In one embodiment, Q of compound of formula II, II(a), III, IV, IV(a), V or V(a) is H. In another embodiment, Q of compound of formula II, II(a), III, IV, IV(a), V or V(a) is F. In another embodiment, Q of compound of formula II, II(a), III, IV, IV(a), V or V(a) is 6-F. In another embodiment, Q of compound of formula II, II(a), III, IV, IV(a), V or V(a) is 5-F. In another embodiment, Q of compound of formula II, II(a), III, IV, IV(a), V or V(a) is Cl. In another embodiment, Q of compound of formula II, II(a), III, IV, IV(a), V or V(a) is 6-Cl. In another embodiment, Q of compound of formula II, II(a), III, IV, IV(a), V or V(a) is 5-Cl. In another embodiment, Q of compound of formula II, II(a), III, IV, IV(a), V or V(a) is Br. In another embodiment, Q of compound of formula II, II(a), III, IV, IV(a), V or V(a) is I. In another embodiment, Q of compound of formula II, II(a), III, IV, IV(a), V or V(a) is OCH₃. In another embodiment, Q of compound of formula II, II(a), III, IV, IV(a), V or V(a) is 6-OCH₃. In another embodiment, Q of compound of formula II, II(a), III, IV, IV(a), V or V(a) is 5-OCH₃. In another embodiment, Q of compound of formula II, II(a), III, IV, IV(a), V or V(a) is CH₃. In another embodiment, Q of compound of formula II, II(a), III, IV, IV(a), V or V(a) is 6-CH₃. In another embodiment, Q of compound of formula II, II(a), III, IV, IV(a), V or V(a) is 5-CH₃. In another embodiment, Q of compound of formula II, II(a), III, IV, IV(a), V or V(a) is located on the C4 position of the fused ring system. In another embodiment, Q of compound of formula II, II(a), III, IV, IV(a), V or V(a) is located on the C5 position of the fused ring system. In another embodiment, Q of compound of formula II, II(a), III, IV, IV(a), V or V(a) is located on the C6 position of the fused ring system. In another embodiment, Q of compound of formula II, II(a), III, IV, IV(a), V or V(a) is located on the C7 position of the fused ring system.

In one embodiment, Z of compound of formula II(a), IV(a) or V(a) is H. In another embodiment, Z of compound of formula II(a), IV(a) or V(a) is F. In another embodiment, Z of compound of formula II(a), IV(a) or V(a) is Cl. In another embodiment, Z of compound of formula II(a), IV(a) or V(a) is Br. In another embodiment, Z of compound of formula II(a), IV(a) or V(a) is I. In another embodiment, Z of compound of formula II(a), IV(a) or V(a) is OCH₃. In another embodiment, Z of compound of formula II(a), IV(a) or V(a) is CH₃. In another embodiment, Z of compound of formula II(a), IV(a) or V(a) is located on the para position of the phenyl. In another embodiment, Z of compound of formula II(a), IV(a) or V(a) is located on the meta position of the phenyl. In another embodiment, Z of compound of formula II(a), IV(a) or V(a) is located on the ortho position of the phenyl.

In one embodiment, R¹ of compound of formula I, II, II(a), III, IV, IV(a), V or V(a) is H. In another embodiment, R¹ of compound of formula I, II, II(a), III, IV, IV(a), V or V(a) is F. In another embodiment, R¹ of compound of formula I, II, II(a), III, IV, IV(a), V or V(a) is 6-F. In another embodiment, R¹ of compound of formula I, II, II(a), III, IV, IV(a), V or V(a) is 5-F. In another embodiment, R¹ of compound of formula I, II, II(a), III, IV, IV(a), V or V(a) is Cl. In another embodiment, R¹ of compound of formula I, II, II(a), III, IV, IV(a), V or V(a) is 6-Cl. In another embodiment, R¹ of compound of formula I, II, II(a), III, IV, IV(a), V or V(a) is 5-Cl. In another embodiment, R¹ of compound of formula I, II, II(a), III, IV, IV(a), V or V(a) is Br. In another embodiment, R¹ of compound of formula I, II, II(a), III, IV, IV(a), V or V(a) is I. In another embodiment, R¹ of compound of formula I, II, II(a), III, IV, IV(a), V or V(a) is OCH₃. In another embodiment, R¹ of compound of formula I, II, II(a), III, IV, IV(a), V or V(a) is 6-OCH₃. In another embodiment, R¹ of compound of formula I, II, II(a), III, IV, IV(a), V or V(a) is 5-OCH₃. In another embodiment, R¹ of compound of formula I, II, II(a), III, IV, IV(a), V or V(a) is CH₃. In another embodiment, R¹ of compound of formula I, II, II(a), III, IV, IV(a), V or V(a) is 6-CH₃. In another embodiment, R¹ of compound of formula I, II, II(a), III, IV, IV(a), V or V(a) is 5-CH₃. In another embodiment, R¹ of compound of formula I, II, II(a), III, IV, IV(a), V or V(a) is located on the C4 position of the fused ring system. In another embodiment, R¹ of compound of formula I, II, II(a), III, IV, IV(a), V or V(a) is located on the C5 position of the fused ring system. In another embodiment, R¹ of compound of formula I, II, II(a), III, IV, IV(a), V or V(a) is located on the C6 position of the fused ring system. In another embodiment, R¹ of compound of formula I, II, II(a), III, IV, IV(a), V or V(a) is located on the C7 position of the fused ring system.

In another embodiment, R¹ of compound of formula I, II, Ill, IV or V is a phenyl substituted at C3 or C5 with R⁴, R⁴ is C(O)R⁶ and R⁶ is a 5- or 6-membered ring independently substituted at C2, C3, C4, C5, C6 or any combination thereof with F.

In one embodiment, n of compound of formula I, II, II(a), III, IV, IV(a), V or V(a) is 0. In another embodiment, n of compound of formula I, II, II(a), III, IV, IV(a), V or V(a) is 1. In another embodiment, n of compound of formula I, II, II(a), III, IV, IV(a), V or V(a) is 2. In another embodiment, n of compound of formula I, II, II(a), III, IV, IV(a), V or V(a) is 3.

In one embodiment, R² of compound of formula I, II, II(a), III, IV, IV(a), V or V(a) is H. In another embodiment, R² of compound of formula I, II, II(a), III, IV, IV(a), V or V(a) is SO₂Ph. In another embodiment, R² of compound of formula I, II, II(a), III, IV, IV(a), V or V(a) is benzyl (Bn). In another embodiment, R² of compound of formula I, II, II(a), III, IV, IV(a), V or V(a) is CH₃.

In one embodiment, R³ of compound of formula I, II, III, IV or V is phenyl substituted at C3 or C5 with R⁴, R⁴ is R⁸R⁹, R⁸ is C═O, and R⁹ is substituted or unsubstituted aryl. In another embodiment, the aryl is substituted at C3, C4 and C5 with OCH₃. In another embodiment, the aryl is substituted at C3 with OCH₃. In another embodiment, the aryl is substituted at C3 and C5 with OCH₃. In another embodiment, the aryl is substituted at C3 and C4 with OCH₃. In another embodiment, the aryl is substituted at C4 with OCH₃. In another embodiment, the aryl is unsubstituted. In another embodiment, the aryl is substituted at C4 with F.

In one embodiment, R³ of compound of formula I, II, III, IV or V is phenyl substituted at C3 or C5 with R⁴, R⁴ is R⁸R⁹, R⁸ is CH(OH), and R⁹ is substituted or unsubstituted aryl. In another embodiment, the aryl is substituted at C3, C4 and C5 with OCH₃. In another embodiment, the aryl is substituted at C3 with OCH₃. In another embodiment, the aryl is substituted at C3 and C5 with OCH₃. In another embodiment, the aryl is substituted at C3 and C4 with OCH₃. In another embodiment, the aryl is substituted at C4 with OCH₃. In another embodiment, the aryl is unsubstituted. In another embodiment, the aryl is substituted at C4 with F.

In one embodiment, R³ of compound of formula I, II, III, IV or V is R⁸R⁹, R⁸ is C═O, and R⁹ is substituted or unsubstituted aryl. In another embodiment, the aryl is substituted at C3, C4 and C5 with OCH₃. In another embodiment, the aryl is substituted at C3 with OCH₃. In another embodiment, the aryl is substituted at C3 and C5 with OCH₃. In another embodiment, the aryl is substituted at C3 and C4 with OCH₃. In another embodiment, the aryl is substituted at C4 with OCH₃. In another embodiment, the aryl is unsubstituted. In another embodiment, the aryl is substituted at C4 with F.

In one embodiment, R³ of compound of formula I, II, III, IV or V is phenyl substituted at C3 or C5 with R⁴; or R⁸R⁹.

In one embodiment, R¹⁰ of compound of formula II(a), IV(a) or V(a) is OCH₃. In another embodiment, R¹⁰ of compound of formula II(a), IV(a) or V(a) is H. In another embodiment, R¹⁰ of compound of formula II(a), IV(a) or V(a) is F. In another embodiment, R¹⁰ of compound of formula II(a), IV(a) or V(a) is Cl. In another embodiment, R¹⁰ of compound of formula II(a), IV(a) or V(a) is Br. In another embodiment, R¹⁰ of compound of formula II(a), IV(a) or V(a) is I.

In one embodiment, m of compound of formula II(a), IV(a) or V(a) is 0. In another embodiment, m of compound of formula II(a), IV(a) or V(a) is 1. In another embodiment, m of compound of formula II(a), IV(a) or V(a) is 2. In another embodiment, m of compound of formula II(a), IV(a) or V(a) is 3. In another embodiment, m of compound of formula II(a), IV(a) or V(a) is 4.

In one embodiment, this invention provides an isomer of compound of formula (I)-V(a). In one embodiment, this invention provides a tautomer of compound of formula (I)-V(a). In another embodiment, this invention provides a pharmaceutically acceptable salt of the compound of formula (I)-V(a). In another embodiment, this invention provides a pharmaceutical product of the compound of formula (I)-V(a). In another embodiment, this invention provides a hydrate of the compound of formula (I)-V(a). In another embodiment, this invention provides an N-oxide of the compound of formula (I)-V(a). In another embodiment, this invention provides any combination of an isomer, tautomer pharmaceutically acceptable salt, pharmaceutical product, hydrate, N-oxide of the compound of formula (I)-V(a).

In some embodiments, the term “isomer” includes, but is not limited to, optical isomers and analogs, structural isomers and analogs, conformational isomers and analogs, stereoisomers, diastereomers, tautomers and the like. In one embodiment, the term “isomer” is meant to encompass optical isomers of the described compounds such as enantiomers and diastereomers. Some compounds may also exhibit polymorphism. It is to be understood that the present invention encompasses any racemic, optically-active, polymorphic, or stereoisomeric form, or mixtures thereof, which form possesses properties useful in the treatment of cancer as described herein.

Compounds of the present invention may exist in the form of one or more of the possible tautomers and depending on the particular conditions it may be possible to separate some or all of the tautomers into individual and distinct entities. It is to be understood that all of the possible tautomers, including all additional enol and keto tautomers and/or isomers are hereby covered.

The invention includes “pharmaceutically acceptable salts” of the compounds of this invention, which may be produced, by reaction of a compound of this invention with an acid or base.

Suitable pharmaceutically-acceptable salts of amines of compounds of this invention may be prepared from an inorganic acid or from an organic acid. In one embodiment, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrate, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In one embodiment, examples of organic salts of amines comprise aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, carboxilates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates, gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoate, hydrofluorate, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, napsylates, N-methylglucamines, oxalates, octanoates, oleates, pamoates, phenylacetates, picrates, phenylbenzoates, pivalates, propionates, phthalates, pectinates, phenylpropionates, palmitates, pantothenates, polygalacturates, pyruvates, quinates, salicylates, succinates, stearates, sulfanilates, subacetates, tartarates, theophyllineacetates, p-toluenesulfonates (tosylates), trifluoroacetates, terephthalates, tannates, teoclates, trihaloacetates, triethiodide, tricarboxylates, undecanoates or valerates.

In one embodiment, examples of inorganic salts of carboxylic acids or phenols comprise ammonium, alkali metals to include lithium, sodium, potassium, cesium; alkaline earth metals to include calcium, magnesium, aluminium; zinc, barium, cholines or quaternary ammoniums.

In another embodiment, examples of organic salts of carboxylic acids or phenols comprise arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolinates, piperazines, procaine, tris(hydroxymethyl)methylamines, triethylamines, triethanolamines, trimethylamines, tromethamines or ureas.

In one embodiment, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

In one embodiment, the invention also includes N-oxides of the amino substituents of the compounds described herein. Also, esters of the phenolic compounds can be made with aliphatic and aromatic carboxylic acids, for example, acetic acid and benzoic acid esters.

This invention provides derivatives of the compounds. In one embodiment, “derivatives” includes but is not limited to ether derivatives, acid derivatives, amide derivatives, ester derivatives and the like. In another embodiment, this invention further includes hydrates of the compounds.

In one embodiment, “hydrate” includes but is not limited to hemihydrate, monohydrate, dihydrate, trihydrate and the like.

This invention provides, in other embodiments, metabolites of the compounds. In one embodiment, “metabolite” means any substance produced from another substance by metabolism or a metabolic process.

This invention provides, in other embodiments, pharmaceutical products of the compounds. The term “pharmaceutical product” refers, in other embodiments, to a composition suitable for pharmaceutical use (pharmaceutical composition), for example, as described herein.

These compounds may be synthesized in any suitable manner. For example, the compounds may be synthesized using the techniques as described in the Examples presented herein.

### Process for the Preparation of Compounds of the Invention.

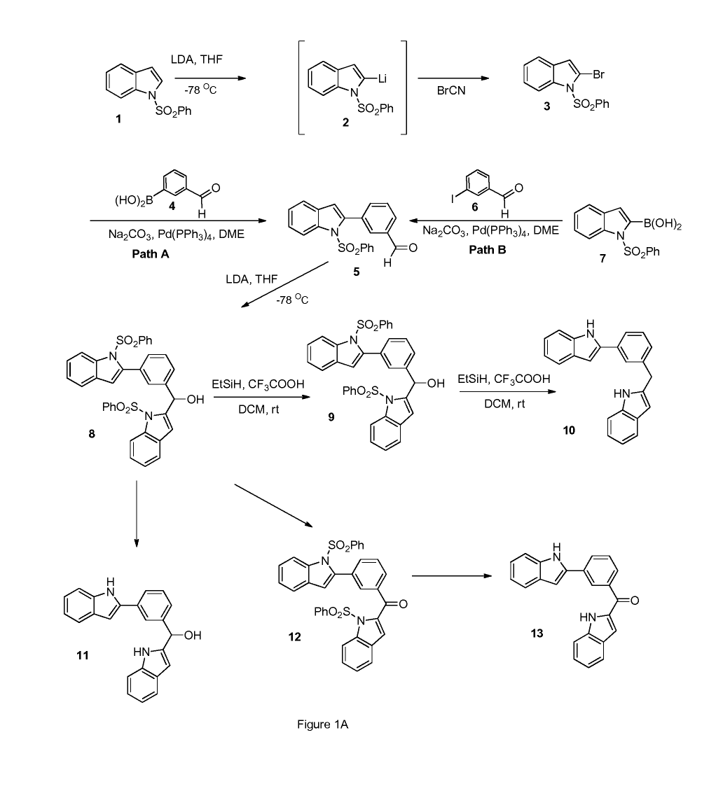

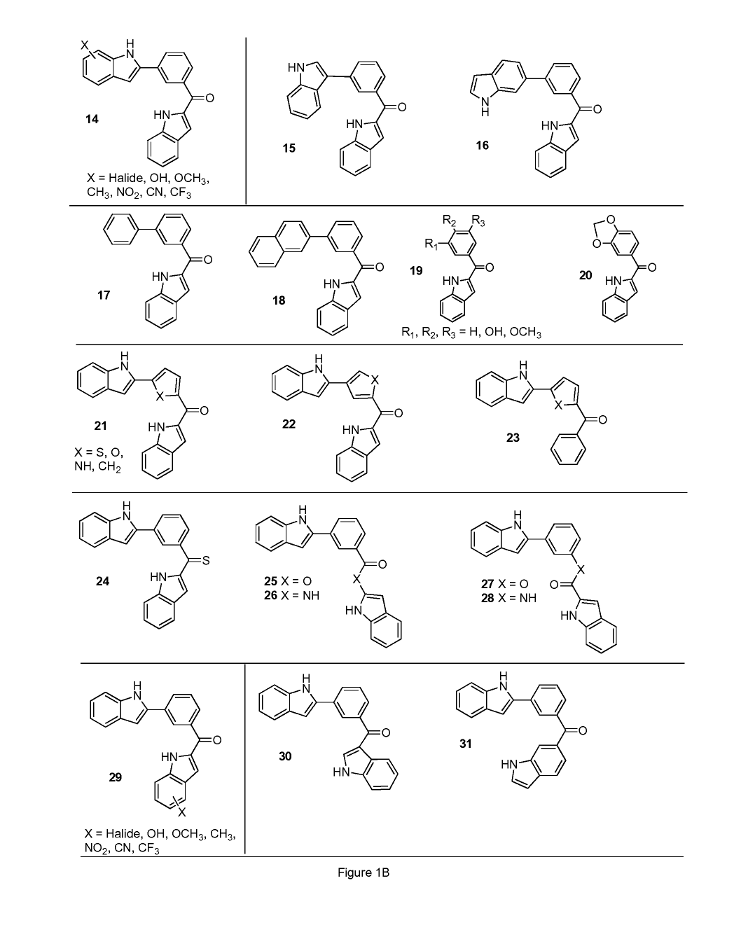

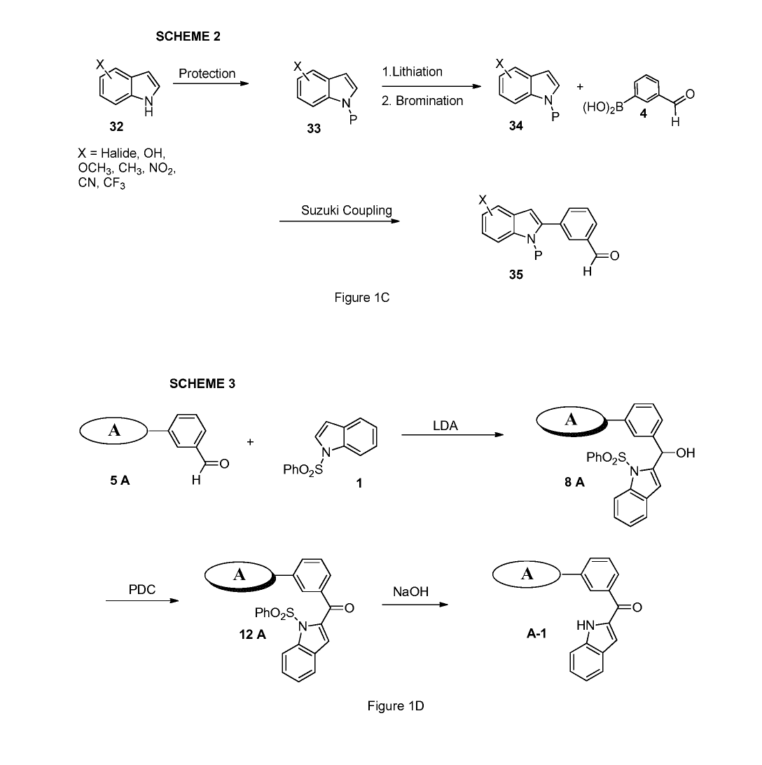

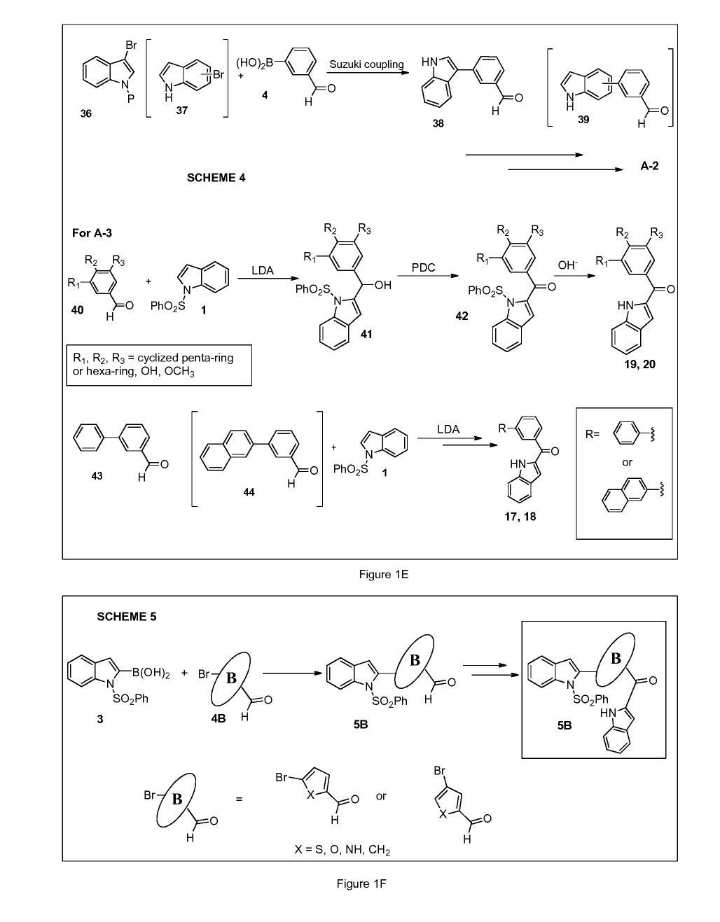

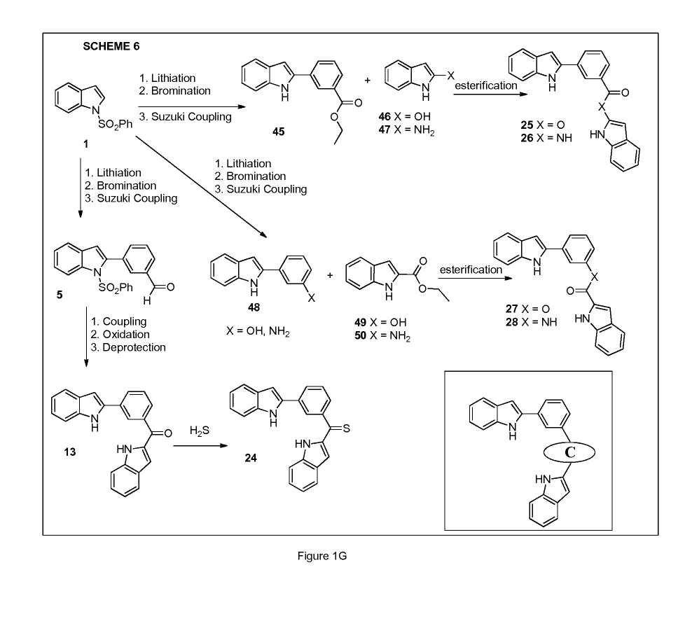

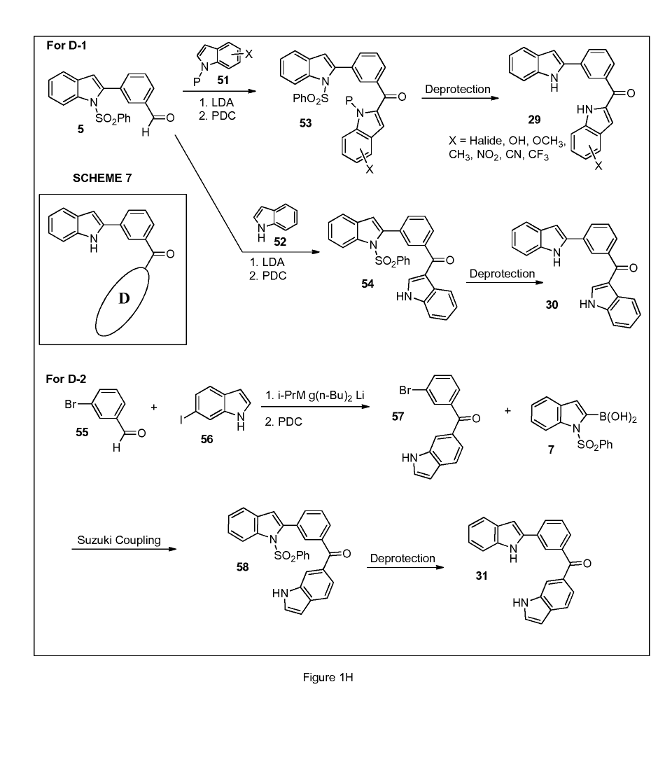

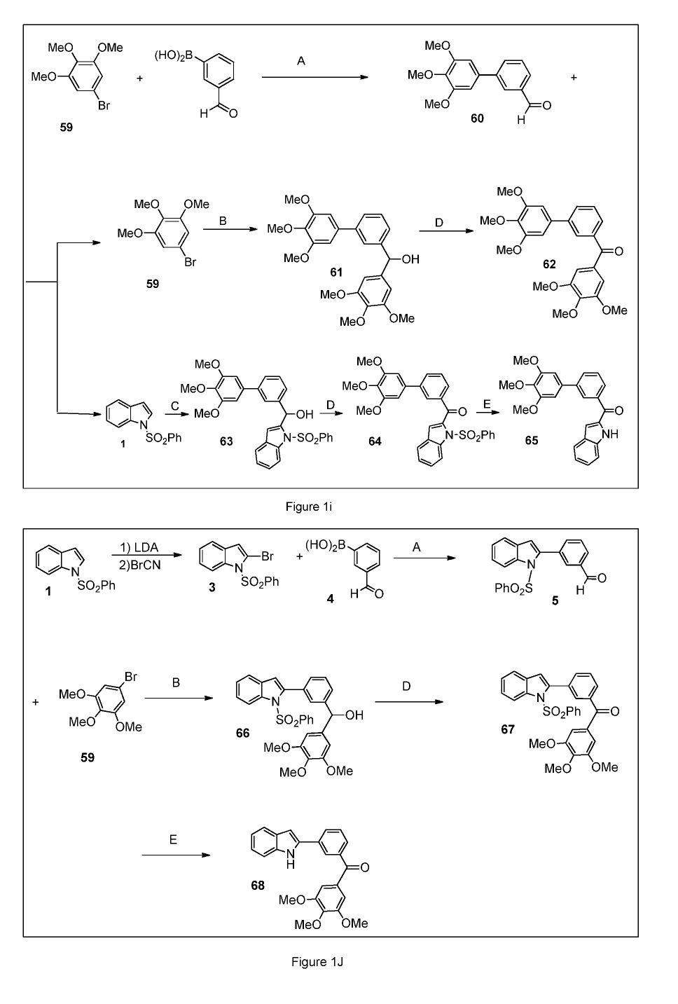

In one embodiment, the present invention provides a process for preparing a compound of this invention, as depicted in FIGS. 1-4 and Examples 1 and 2.

In one embodiment, the present invention provides process (1) for preparing a compound represented by the structure of formula II(a):

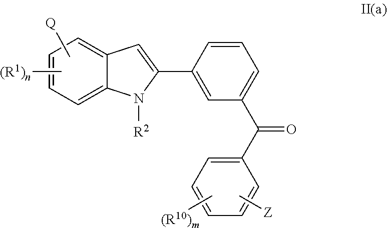

said process comprising the steps of:
a) coupling an indole bromide derivative of formula X:

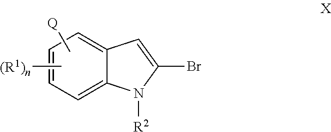

with an arylboronic acid of formula XI:

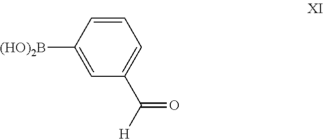

under Suzuki coupling conditions, to produce the aldehyde of formula XII:

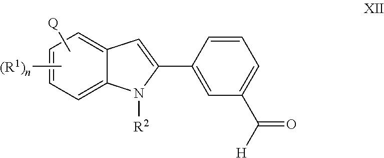

b) treating the aldehyde of formula XII with an arylbromide derivative of formula XIII:

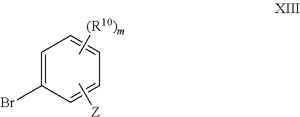

to produce the corresponding intermediate of formula XIV:

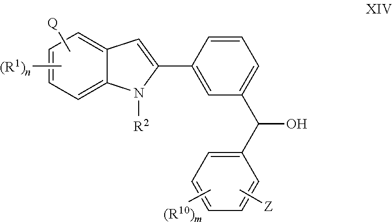

and:
c) oxidizing the intermediate of formula XIV to obtain the respective ketone of formula II(a):

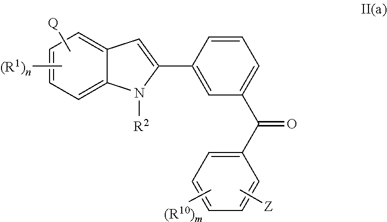

wherein R² is SO₂Ph;
(d) optionally hydrolyzing compound of formula II(a) to afford a compound of formula II(a):

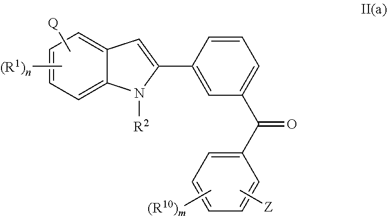

wherein R² is H;
(e) optionally treating the resulting compound with an alkylating agent to produce the N-alkylated compound of formula II(a):

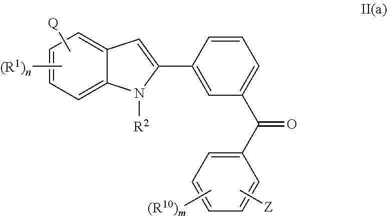

wherein R² is alkyl, CH₃ or benzyl, and R¹, Q, R¹⁰, Z, n and m are as defined hereinabove for compound of formula II(a).

In one embodiment the conditions of step (a) of process (1) for preparing a compound of formula II(a) outlined hereinabove comprises a coupling agent. In another embodiment, the coupling agent comprises Pd(0) catalyst. In another embodiment the palladium catalyst is Pd(PPh₃)₄. In another embodiment, the reaction is done in the presence of a base. Nonlimiting examples of bases are carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), alkali metal hydrides such as sodium hydride (NaH), potassium hydride (KH) and lithium hydride (LiH), and the like. In another embodiment, the base is potassium carbonate (K₂CO₃). In one embodiment, the reaction is carried out at an appropriate temperature, as will be known to one skilled in the art, for example, in the range, of −20 to 120° C., or for example at or near ambient temperature. In another embodiment, the resulting mixture is refluxed. In another embodiment, the conditions of step (a) of the process (1) for preparing a compound of formula II(a) outlined hereinabove comprises a solvent. Non limiting examples for a suitable solvent or diluent are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment the solvent is DMF.

In one embodiment, step (b) of process (1) for preparing a compound of formula II(a) outlined hereinabove comprises a base. Nonlimiting examples of bases are alkyl lithium bases such as butyl lithium (BuLi), phenyl lithium (PhLi), benzyl lithium (BnLi), methyl lithium (MeLi); carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), alkali metal hydrides such as sodium hydride (NaH), potassium hydride (KH) and lithium hydride (LiH), and the like. In another embodiment, the base is BuLi. In another embodiment, the reaction is carried out under inert atmosphere. In another embodiment, the reaction is carried out at an appropriate temperature, as will be known to one skilled in the art, for example, in the range, of −20 to 120° C., or for example at or near ambient temperature. In another embodiment, the reaction is done at low temperature. In another embodiment, the reaction is done at −78° C. In another embodiment, the resulting mixture is refluxed under inert atmosphere. In another embodiment, the resulting mixture is allowed to warm to rt under inert atmosphere. In another embodiment, the conditions of step (b) of the process (1) for preparing a compound of formula II(a) outlined hereinabove comprises a solvent, low temperature and inert atmosphere. Non limiting examples for a suitable solvent or diluent are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is an anhydrous solvent. In another embodiment the solvent is THF. In another embodiment, following the reaction, the reaction mixture is quenched. In another embodiment, the reaction mixture is quenched with NaHCO₃.

In one embodiment the conditions of step (c) of process (1) for preparing a compound of formula II(a) outlined hereinabove comprises an oxidation agent. In another embodiment, the oxidation agent comprises ozone. In another embodiment, the oxidizing agent is a peroxyacid, for example, peroxyacetic acid, (CH₃COOOH). In another embodiment, the oxidizing agent is pyridinium dichromate (PDC). In another embodiment, the oxidizing agent is Dess-Martin periodinate. In another embodiment, the oxidizing agent is meta-chloroperoxybenzoic acid (m-CPBA). In another embodiment, the oxidizing agent is Magnesium MonoPeroxyPhthalic Acid (MMPP). Non limiting examples for a suitable solvent or diluent for this reaction are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment, the solvent of is DMF. In another embodiment the solvent is CH₂Cl₂. In another embodiment, the reaction is carried out at rt.

In another embodiment, the conditions of step (d) of process (1) for preparing a compound of formula II(a) outlined hereinabove comprises a base. In another embodiment, the base comprises hydroxides such as alkali hydroxides, for example sodium hydroxide (NaOH), potassium hydroxide (KOH) and cesium hydroxide (CsOH); carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), alkali metal hydrides such as sodium hydride (NaH), potassium hydride (KH) and lithium hydride (LiH), and the like, or another base appropriate for this reaction. In another embodiment, the base is NaOH. In another embodiment, the base is KOH. Non limiting examples for a suitable solvent or diluent for this reaction are, for example, tetrahydrofuran (THF), water, ethanol (EtOH), methanol (MeOH), isopropanol (iPrOH), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment, the solvent of is EtOH, water, or combination thereof.

In another embodiment, the alkylating agent used in step (e) of process (1) for preparing a compound of formula II(a) outlined hereinabove comprises methyl iodide. In another embodiment, the alkylating agent comprises is benzyl bromide. In another embodiment, the reaction comprises a base. In another embodiment, the base comprises alkali metal hydrides such as sodium hydride (NaH), potassium hydride (KH) and lithium hydride (LiH), and the like; hydroxides such as alkali hydroxides, for example sodium hydroxide (NaOH), potassium hydroxide (KOH) and cesium hydroxide (CsOH); carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), or another base appropriate for this reaction. In another embodiment, the base is NaH. Non limiting examples for a suitable solvent or diluent for this reaction are, for example, tetrahydrofuran (THF), water, ethanol (EtOH), methanol (MeOH), isopropanol (iPrOH), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment, the solvent of is THF. In another embodiment, the reaction is carried out at an appropriate temperature, as will be known to one skilled in the art, for example, in the range, of −20 to 120° C., or for example at or near ambient temperature. In another embodiment, the reaction is carried out at 0° C. In another embodiment, the reaction is carried out under inert atmosphere. In another embodiment, following the addition of the alkylation agent, the reaction mixture is refluxed.

In one embodiment, the present invention provides process (2) for preparing a compound represented by the structure of formula II(a):

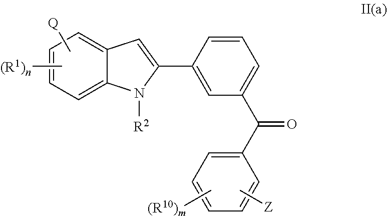

said process comprising the steps of:
a) coupling an indole bromide derivative of formula X:

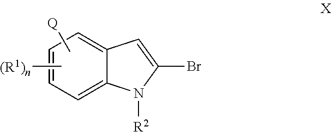

with an arylboronic acid of formula XI:

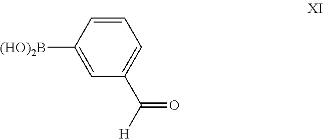

under Suzuki coupling conditions, to produce the aldehyde of formula XII:

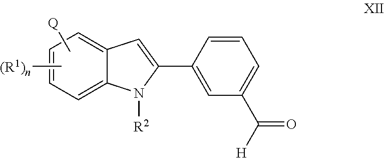

b) treating the aldehyde of formula XII with an aryl-Grignard reagent of formula XV:

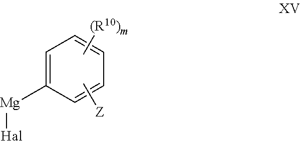

wherein Hal refers to a halide
to produce the corresponding intermediate of formula XIV

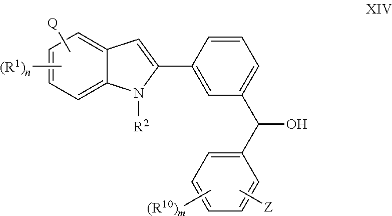

and:
c) oxidizing the intermediate of formula XIV to form the respective ketone of formula II(a):

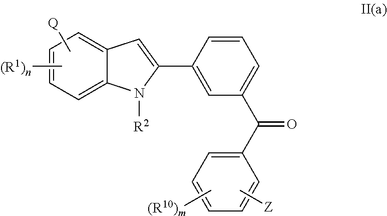

wherein R² is SO₂Ph;
(d) optionally hydrolyzing compound of formula II(a) to afford a compound of formula II(a):

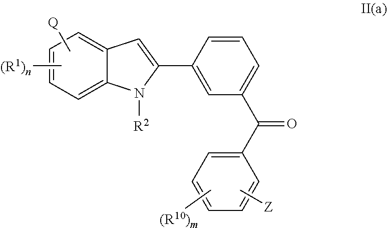

wherein R² is H;
(e) optionally treating the resulting compound with an alkylating agent to produce the N-alkylated compound of formula II(a):

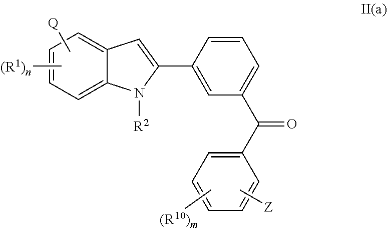

wherein R² is alkyl, CH₃ or benzyl, and R¹, Q, R¹⁰, Z, n and m are as defined hereinabove for compound of formula II(a).

In one embodiment the conditions of step (a) of process (2) for preparing a compound of formula II(a) outlined hereinabove comprises a coupling agent. In another embodiment, the coupling agent comprises Pd(0) catalyst. In another embodiment the Paladium catalyst is Pd(PPh₃)₄. In another embodiment, the reaction is done in the presence of a base. Nonlimiting examples of bases are carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), alkali metal hydrides such as sodium hydride (NaH), potassium hydride (KH) and lithium hydride (LiH), and the like. In another embodiment, the base is potassium carbonate (K₂CO₃). In one embodiment, the reaction is carried out at an appropriate temperature, as will be known to one skilled in the art, for example, in the range, of −20 to 120° C., or for example at or near ambient temperature. In another embodiment, the resulting mixture is refluxed. In another embodiment, the conditions of step (a) of the process (2) for preparing a compound of formula II(a) outlined hereinabove comprises a solvent. Non limiting examples for a suitable solvent or diluent are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment the solvent is DMF.

In one embodiment, step (b) of process (2) for preparing a compound of formula II(a) outlined hereinabove is carried out at an appropriate temperature, as will be known to one skilled in the art, for example, in the range, of −20 to 120° C., or for example at or near ambient temperature. In another embodiment, the reaction is done at room temperature (rt). In another embodiment, the reaction is done at −78° C. In another embodiment, the reaction is carried out under inert atmosphere. In another embodiment, the resulting mixture is refluxed under inert atmosphere. In another embodiment, the resulting mixture is allowed to warm to rt under inert atmosphere. In another embodiment, the conditions of step (b) of the process (2) for preparing a compound of formula II(a) outlined hereinabove comprises a solvent. Non limiting examples for a suitable solvent or diluent are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment the solvent is THF. In another embodiment, following the reaction, the reaction mixture is quenched. In another embodiment, the reaction mixture is quenched with NaHCO₃.

In one embodiment the conditions of step (c) of process (2) for preparing a compound of formula II(a) outlined hereinabove comprises an oxidation agent. In another embodiment, the oxidation agent comprises ozone. In another embodiment, the oxidizing agent is a peroxyacid, for example, peroxyacetic acid, (CH₃COOOH). In another embodiment, the oxidizing agent is pyridinium dichromate (PDC). In another embodiment, the oxidizing agent is Dess-Martin periodinate. In another embodiment, the oxidizing agent is meta-chloroperoxybenzoic acid (m-CPBA). In another embodiment, the oxidizing agent is Magnesium MonoPeroxyPhthalic Acid (MMPP). Non limiting examples for a suitable solvent or diluent for this reaction are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment, the solvent of is DMF. In another embodiment, the reaction is carried out at rt.

In another embodiment, the conditions of step (d) of process (2) for preparing a compound of formula II(a) outlined hereinabove comprises a base. In another embodiment, the base comprises hydroxides such as alkali hydroxides, for example sodium hydroxide (NaOH), potassium hydroxide (KOH) and cesium hydroxide (CsOH); carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), alkali metal hydrides such as sodium hydride (NaH), potassium hydride (KH) and lithium hydride (LiH), and the like, or another base appropriate for this reaction. In another embodiment, the base is NaOH. Non limiting examples for a suitable solvent or diluent for this reaction are, for example, tetrahydrofuran (THF), water, ethanol (EtOH), methanol (MeOH), isopropanol (iPrOH), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment, the solvent of is EtOH, water, or combination thereof.

In another embodiment, the alkylating agent used in step (e) of process (2) for preparing a compound of formula II(a) outlined hereinabove comprises methyl iodide. In another embodiment, the alkylating agent comprises is benzyl bromide. In another embodiment, the reaction comprises a base. In another embodiment, the base comprises alkali metal hydrides such as sodium hydride (NaH), potassium hydride (KH) and lithium hydride (LiH), and the like; hydroxides such as alkali hydroxides, for example sodium hydroxide (NaOH), potassium hydroxide (KOH) and cesium hydroxide (CsOH); carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), or another base appropriate for this reaction. In another embodiment, the base is NaH. Non limiting examples for a suitable solvent or diluent for this reaction are, for example, tetrahydrofuran (THF), water, ethanol (EtOH), methanol (MeOH), isopropanol (iPrOH), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment, the solvent of is THF. In another embodiment, the reaction is carried out at an appropriate temperature, as will be known to one skilled in the art, for example, in the range, of −20 to 120° C., or for example at or near ambient temperature. In another embodiment, the reaction is carried out at 0° C. In another embodiment, the reaction is carried out under inert atmosphere. In another embodiment, following the addition of the alkylation agent, the reaction mixture is refluxed.

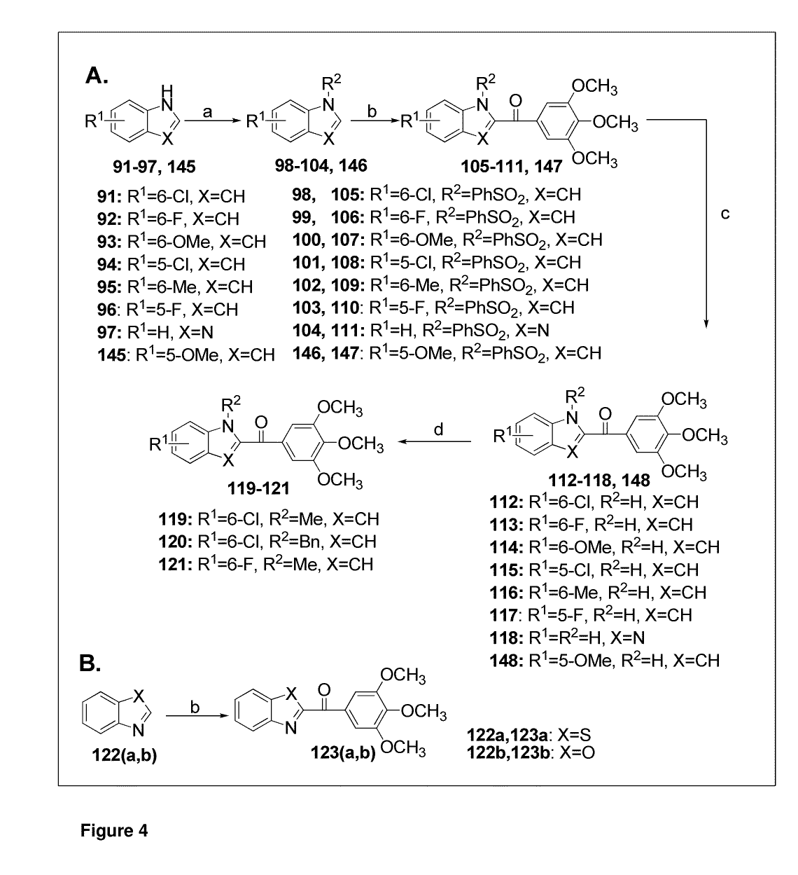

In one embodiment, the present invention provides a process for preparing a compound of this invention, as depicted in FIG. 4 and Example 1.

In another embodiment, the present invention provides a process for preparing a compound represented by the structure of formula IV(a):

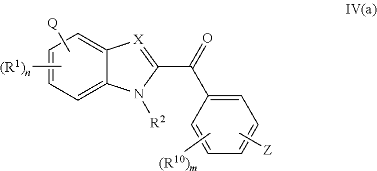

wherein X is N or CH and R² is SO₂Ph;
said process comprising the steps of:
a) protection of the amine of a compound of formula XX:

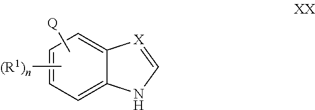

with a benzenesulfonyl (—SO₂Ph) chloride to yield the corresponding protected compound of formula XXI:

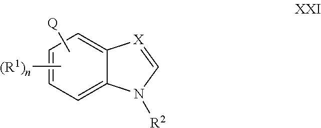

(b) deprotonation of compound XXI
followed by
(c) coupling with a benzoyl chloride derivative of formula XXII:

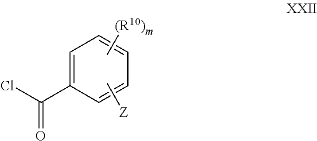

to afford a compound of formula IV(a):

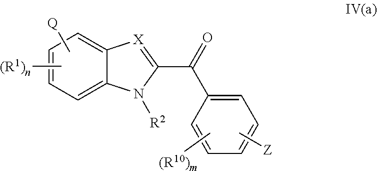

(d) optionally hydrolyzing the protected compound of formula IV(a) to afford a compound of formula IV(a):

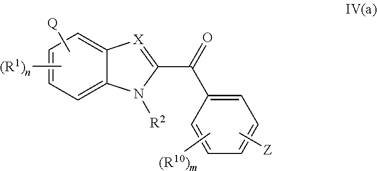

wherein R² is H; and
(e) optionally treating the resulting compound with an alkylating agent to produce the N-alkylated compound of formula IV(a)

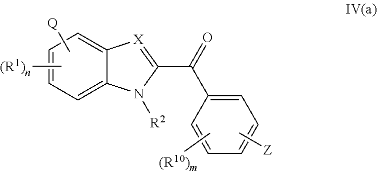

wherein R² is alkyl, CH₃ or benzyl, and R¹, Q, R¹⁰, Z, n and m are as defined hereinabove for compound of formula IV(a).

In one embodiment the conditions of step (a) of the process for preparing a compound of formula IV(a) outlined hereinabove comprises a base. Nonlimiting examples of bases are alkali metal hydrides such as sodium hydride (NaH), potassium hydride (KH) and lithium hydride (LiH); carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), and the like. In another embodiment, the base is sodium hydride (NaH). In one embodiment, the reaction is carried out at an appropriate temperature, as will be known to one skilled in the art, for example, in the range, of −20 to 120° C., or for example at or near ambient temperature. In another embodiment, the reaction is carried out at 0° C. In another embodiment, the conditions of step (a) of the process for preparing a compound of formula IV(a) outlined hereinabove comprises a solvent. Non limiting examples for a suitable solvent or diluent are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment the solvent is THF.

In one embodiment, step (b) of the process for preparing a compound of formula IV(a) outlined hereinabove comprises a deprotonation agent. In another embodiment, the deprotonated agent is a lithium reagent. In another embodiment, the lithium reagent is tert-butyllithium. In another embodiment, the reaction is carried out at an appropriate temperature, as will be known to one skilled in the art, for example, in the range, of −20 to 120° C., or for example at or near ambient temperature. In another embodiment, the reaction is carried out at −78° C. In another embodiment, the reaction is carried out under inert atmosphere. In another embodiment, the conditions of step (b) comprise a solvent. Non limiting examples for a suitable solvent or diluent are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment the solvent is THF.

In one embodiment the conditions of step (c) of the process for preparing a compound of formula IV(a) outlined hereinabove comprise a solvent. Non limiting examples for a suitable solvent or diluent for this reaction are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment, the solvent of is THF. In another embodiment, the reaction is carried out at an appropriate temperature, as will be known to one skilled in the art, for example, in the range, of −20 to 120° C., or for example at or near ambient temperature. In another embodiment, the reaction is carried out at −78° C. In another embodiment, the reaction is carried out under inert atmosphere.

In another embodiment, the conditions of step (d) of the process for preparing a compound of formula IV(a) outlined hereinabove comprise a base. In another embodiment, the base comprises hydroxides such as alkali hydroxides, for example sodium hydroxide (NaOH), potassium hydroxide (KOH) and cesium hydroxide (CsOH); carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), alkali metal hydrides such as sodium hydride (NaH), potassium hydride (KH) and lithium hydride (LiH), and the like, or another base appropriate for this reaction. In another embodiment, the base is NaOH. In another embodiment the reaction is carried out in the darkness. Non limiting examples for a suitable solvent or diluent for this reaction are, for example, tetrahydrofuran (THF), water, ethanol (EtOH), methanol (MeOH), isopropanol (iPrOH), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment, the solvent of is EtOH, water or combination thereof.

In another embodiment, the alkylating agent used in step (e) of the process for preparing a compound of formula IV(a) outlined hereinabove comprises methyl iodide. In another embodiment, the alkylating agent comprises benzyl bromide. In another embodiment, the reaction comprises a base. In another embodiment, the base comprises alkali metal hydrides such as sodium hydride (NaH), potassium hydride (KH) and lithium hydride (LiH), and the like; hydroxides such as alkali hydroxides, for example sodium hydroxide (NaOH), potassium hydroxide (KOH) and cesium hydroxide (CsOH); carbonates such as alkali carbonates, for example sodium carbonate (Na₂CO₃), potassium carbonate (K₂CO₃) and cesium carbonate (Cs₂CO₃); bicarbonates such as alkali metal bicarbonates, for example sodium bicarbonate (NaHCO₃), potassium bicarbonate (KHCO₃), or another base appropriate for this reaction. In another embodiment, the base is NaH. Non limiting examples for a suitable solvent or diluent for this reaction are, for example, tetrahydrofuran (THF), water, ethanol (EtOH), methanol (MeOH), isopropanol (iPrOH), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment, the solvent of is THF. In another embodiment, the reaction is carried out at an appropriate temperature, as will be known to one skilled in the art, for example, in the range, of −20 to 120° C., or for example at or near ambient temperature. In another embodiment, the reaction is carried out at 0° C. In another embodiment, the reaction is carried out under inert atmosphere. In another embodiment, following the addition of the alkylation agent, the reaction mixture is refluxed.

In another embodiment, the present invention provides a process for preparing a compound represented by the structure of formula V(a):

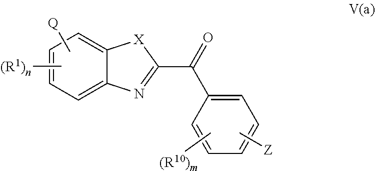

wherein X is O or S;
said process comprising the steps of:
(a) deprotonation of compound XXX:

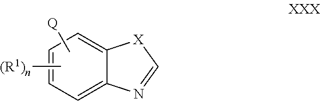

following by:
(b) coupling with a benzoyl chloride derivative of formula XXII:

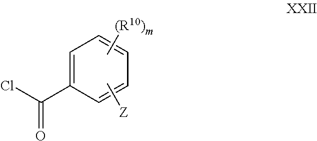

to afford a compound of formula V(a):

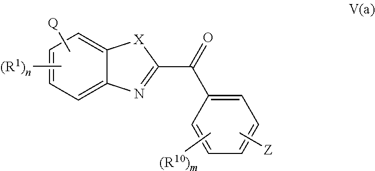

wherein R¹, Q, R¹⁰, Z, n and m are as defined hereinabove for compound of formula V(a).

In one embodiment the conditions of step (a) of the process for preparing a compound of formula V(a) outlined hereinabove comprises a deprotonation agent. In another embodiment, the deprotonated agent is a lithium reagent. In another embodiment, the lithium reagent is tert-butyllithium. In another embodiment, the reaction is carried out at an appropriate temperature, as will be known to one skilled in the art, for example, in the range, of −20 to 120° C., or for example at or near ambient temperature. In another embodiment, the reaction is carried out at −78° C. In another embodiment, the reaction is carried out under inert atmosphere. In another embodiment, the conditions of step (a) comprise a solvent. Non limiting examples for a suitable solvent or diluent are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment the solvent is THF.

In one embodiment the conditions of step (b) of the process for preparing a compound of formula V(a) outlined hereinabove comprise a solvent. Non limiting examples for a suitable solvent or diluent for this reaction are, for example, tetrahydrofuran (THF), diethyl ether, acetone, methyl ethyl ketone, 2-propanol, aromatic amines such as pyridine; aliphatic and aromatic hydrocarbons such as benzene, toluene, and xylene; dimethylsulfoxide (DMSO), dimethylformamide (DMF), and dimethylacetamide (DMAC). In another embodiment the solvent is anhydrous solvent. In another embodiment, the solvent of is THF. In another embodiment, the reaction is carried out at an appropriate temperature, as will be known to one skilled in the art, for example, in the range, of −20 to 120° C., or for example at or near ambient temperature. In another embodiment, the reaction is carried out at −78° C. In another embodiment, the reaction is carried out under inert atmosphere.

### Pharmaceutical Compositions

In some embodiments, this invention provides methods of use which comprise administering a composition comprising the described compounds. As used herein, “pharmaceutical composition” means a “therapeutically effective amount” of the active ingredient, i.e. the compound of the invention, together with a pharmaceutically acceptable carrier or diluent. A “therapeutically effective amount” as used herein refers to that amount which provides a therapeutic effect for a given condition and administration regimen.

As used herein, the term “administering” refers to bringing a subject in contact with a compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

The pharmaceutical compositions containing the compounds of this invention can be administered to a subject by any method known to a person skilled in the art, such as orally, parenterally, intravascularly, paracancerally, transmucosally, transdermally, intramuscularly, intranasally, intravenously, intradermally, subcutaneously, sublingually, intraperitoneally, intraventricularly, intracranially, intravaginally, by inhalation, rectally, intratumorally, or by any means in which a recombinant virus/composition can be delivered to tissue (e.g., needle or catheter). Alternatively, topical administration may be desired for application to mucosal cells, for skin or ocular application. Another method of administration is via aspiration or aerosol formulation.

In one embodiment, the pharmaceutical compositions are administered orally, and are thus formulated in a form suitable for oral administration, i.e. as a solid or a liquid preparation. Suitable solid oral formulations include tablets, capsules, pills, granules, pellets, powders, and the like. Suitable liquid oral formulations include solutions, suspensions, dispersions, emulsions, oils and the like. In one embodiment of the present invention, the compounds are formulated in a capsule. In accordance with this embodiment, the compositions of the present invention comprise in addition to a compound of this invention and the inert carrier or diluent, a hard gelatin capsule.

In one embodiment, the micronized capsules comprise particles containing a compound of this invention, wherein the term “micronized” used herein refers to particles having a particle size of less than 100 microns, or in another embodiment, less than 60 microns, or in another embodiment, less than 36 microns, or in another embodiment, less than 16 microns, or in another embodiment, less than 10 microns, or in another embodiment, less than 6 microns.

Further, in another embodiment, the pharmaceutical compositions are administered by intravenous, intraarterial, or intramuscular injection of a liquid preparation. Suitable liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intraarterially, and are thus formulated in a form suitable for intraarterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, the compounds of this invention or their physiologically tolerated derivatives such as salts, esters, N-oxides, and the like are prepared and applied as solutions, suspensions, or emulsions in a physiologically acceptable diluent with or without a pharmaceutical carrier.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of a compound as herein described over a period of time. In a further embodiment, the pharmaceutical compositions are administered intravaginally.

In another embodiment, the active compound can be delivered in a vesicle, in particular a liposome (see Langer, Science 249:1627-1633 (1990); Treat et al., in Liposomes in the Therapy of Infectious Disease and Cancer, Lopez-Berestein and Fidler (eds.), Liss, New York, pp. 363-366 (1989); Lopez-Berestein, ibid., pp. 317-327; see generally ibid).

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In one embodiment, the compositions of this invention may include, a compound of this invention or any combination thereof, together with one or more pharmaceutically acceptable excipients.

It is to be understood that this invention encompasses any embodiment of a compound as described herein, which in some embodiments is referred to as “a compound of this invention”.

Suitable excipients and carriers may be, according to embodiments of the invention, solid or liquid and the type is generally chosen based on the type of administration being used. Liposomes may also be used to deliver the composition. Examples of suitable solid carriers include lactose, sucrose, gelatin and agar. Oral dosage forms may contain suitable binders, lubricants, diluents, disintegrating agents, coloring agents, flavoring agents, flow-inducing agents, and melting agents. Liquid dosage forms may contain, for example, suitable solvents, preservatives, emulsifying agents, suspending agents, diluents, sweeteners, thickeners, and melting agents. Parenteral and intravenous forms should also include minerals and other materials to make them compatible with the type of injection or delivery system chosen. Of course, other excipients may also be used.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, cyclodextrins, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzalkonium chloride, benzyl benzoate, cyclodextrins, sobitan esters, stearic acids), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweetners (e.g. aspartame, citric acid), preservatives (e.g., Thimerosal, benzyl alcohol, parabens), coloring agents, lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates), and/or adjuvants.

In one embodiment, the pharmaceutical compositions provided herein are controlled release compositions, i.e. compositions in which the compound of this invention is released over a period of time after administration. Controlled or sustained release compositions include formulation in lipophilic depots (e.g. fatty acids, waxes, oils). In another embodiment, the composition is an immediate release composition, i.e. a composition in which all of the compound is released immediately after administration.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:607 (1980); Saudek et al., N. Engl. J. Med. 321:674 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 116-138 (1984). Other controlled release systems are discussed in the review by Langer (Science 249:1627-1633 (1990)).

The compositions may also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts adducts less frequently or in lower doses than with the unmodified compound.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, the compounds of this invention or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the compounds of this invention or their physiologically tolerated derivatives such as salts, esters, N-oxides, and the like are converted into a solution, suspension, or emulsion, if desired with the substances customary and suitable for this purpose, for example, solubilizers or other.

An active component can be formulated into the composition as neutralized pharmaceutically acceptable salt forms. Pharmaceutically acceptable salts include the acid addition salts, which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of the compound will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid.

In one embodiment, this invention provides pharmaceutical compositions comprising a compound of this invention. In one embodiment, such compositions are useful for treating cancer in a subject.

In one embodiment, this invention also provides a composition comprising two or more compounds of this invention, or polymorphs, isomers, hydrates, salts, or N-oxides thereof. The present invention also relates to compositions and pharmaceutical compositions which comprise a compound of this invention alone or in combination with a chemotherapeutic compound, osteogenic or myogenic compound, or other agents suitable for the applications as herein described. In one embodiment, the compositions of this invention will comprise a suitable carrier, diluent or salt.

In one embodiment, the methods of this invention may comprise administration of a compound of formula I-V(a) of this invention at various dosages. In one embodiment, the compound of this invention is administered at a dosage of 0.01-200 mg per day. In one embodiment, the compound of this invention is administered at a dose of 0.1-10 mg per day, or in another embodiment, 0.1-26 mg per day, or in another embodiment, 0.1-60 mg per day, or in another embodiment, 0.3-16 mg per day, or in another embodiment, 0.3-30 mg per day, or in another embodiment, 0.6-26 mg per day, or in another embodiment, 0.6-60 mg per day, or in another embodiment, 0.76-16 mg per day, or in another embodiment, 0.76-60 mg per day, or in another embodiment, 1-6 mg per day, or in another embodiment, 1-20 mg per day, or in another embodiment, 3-16 mg per day, or in another embodiment, 30-60 mg per day, or in another embodiment, 30-76 mg per day, or in another embodiment, 100-2000 mg per day. In one embodiment, the compound of this invention is administered at a dosage of 1 mg per day. In another embodiment the compound of this invention is administered at a dosage of 6 mg, 10 mg, 16 mg, 20 mg, 26 mg, 30 mg, 36 mg, 40 mg, 46 mg, 50 mg, 56 mg, 60 mg, 66 mg, 70 mg, 76 mg, 80 mg, 86 mg, 90 mg, 96 mg or 100 mg per day.

In one embodiment, the methods of this invention may comprise administration of a compound of formula I-V(a) of this invention at various dosages. In one embodiment, the compound of this invention is administered at a dosage of 0.01-2000 mg per day. In one embodiment, the compound of this invention is administered at a dose of 0.1-10 mg per kg, or in another embodiment, 0.1-26 mg per kg, or in another embodiment, 0.1-60 mg per kg, or in another embodiment, 0.3-16 mg per kg, or in another embodiment, 0.3-30 mg per kg, or in another embodiment, 0.6-26 mg per kg, or in another embodiment, 0.6-60 mg per kg, or in another embodiment, 0.76-16 mg per day, or in another embodiment, 0.76-60 mg per kg, or in another embodiment, 1-6 mg per kg, or in another embodiment, 1-20 mg per kg, or in another embodiment, 3-16 mg per day, or in another embodiment, 30-60 mg per kg, or in another embodiment, 30-76 mg per kg, or in another embodiment, 100-2000 mg per kg. In one embodiment, the compound of this invention is administered at a dosage of 1 mg per kg. In another embodiment the compound of this invention is administered at a dosage of 6 mg, 10 mg, 16 mg, 20 mg, 26 mg, 30 mg, 36 mg, 40 mg, 46 mg, 50 mg, 56 mg, 60 mg, 66 mg, 70 mg, 76 mg, 80 mg, 86 mg, 90 mg, 96 mg or 100 mg per kg.

Dosage formulations of these compounds or a pharmacologically acceptable salt or hydrate thereof may comprise conventional non-toxic, physiologically or pharmaceutically acceptable carriers or vehicles suitable for the method of administration. These compounds or pharmaceutical compositions thereof may be administered independently one or more times to achieve, maintain or improve upon a pharmacologic or therapeutic effect derived from these compounds or other anticancer drugs or agents. It is well within the skill of an artisan to determine dosage or whether a suitable dosage comprises a single administered dose or multiple administered doses. An appropriate dosage depends on the subject's health, the progression or remission of the cancer, the route of administration and the formulation used.

For administration to mammals, and particularly humans, it is expected that the physician will determine the actual dosage and duration of treatment, which will be most suitable for an individual and can vary with the age, weight and response of the particular individual.

In one embodiment, the present invention provides methods of use comprising the administration of a pharmaceutical composition comprising a) any embodiment of a compound as described herein; and b) a pharmaceutically acceptable carrier or diluent; which is to be understood to include an isomer, tautomer, pharmaceutically acceptable salt, N-oxide, hydrate or any combination thereof of a compound as herein described, and may comprise compounds of formula I-V(a).

In some embodiments, the present invention provides methods of use of a pharmaceutical composition comprising a) any embodiment of the compounds as described herein, including an isomer, tautomer, pharmaceutically acceptable salt, pharmaceutical product, N-oxide, hydrate, or any combination thereof; b) a pharmaceutically acceptable carrier or diluent; c) a flow-aid; and d) a lubricant.

In another embodiment, the present invention provides methods of use of a pharmaceutical composition comprising a) any embodiment of the compounds as described herein, including an isomer, tautomer, pharmaceutically acceptable salt, pharmaceutical product, N-oxide, hydrate or any combination thereof; b) lactose monohydrate; c) microcrystalline cellulose; d) magnesium stearate; and e) colloidal silicon dioxide.

In some embodiments, the methods of this invention make use of compositions comprising compounds of this invention, which offer the advantage that the compounds possess anti proliferative properties in vivo. According to this aspect, such compounds are unaccompanied by serious side effects, provide convenient modes of administration, and lower production costs and are orally bioavailable, lack significant cross-reactivity with other receptors, and may possess long biological half-lives.

In one embodiment, the compositions for administration may be sterile solutions, or in other embodiments, aqueous or non-aqueous, suspensions or emulsions. In one embodiment, the compositions may comprise propylene glycol, polyethylene glycol, injectable organic esters, for example ethyl oleate, or cyclodextrins. In another embodiment, compositions may also comprise wetting, emulsifying and/or dispersing agents. In another embodiment, the compositions may also comprise sterile water or any other sterile injectable medium.

In one embodiment, the invention provides compounds and compositions, including any embodiment described herein, for use in any of the methods of this invention, as described herein. In one embodiment, use of a compound of this invention or a composition comprising the same, will have utility in inhibiting, suppressing, enhancing or stimulating a desired response in a subject, as will be understood by one skilled in the art. In another embodiment, the compositions may further comprise additional active ingredients, whose activity is useful for the particular application for which the compound of this invention is being administered.

In some embodiments, the composition will comprise the compounds as described herein, as well as another therapeutic compound, including inter alia, a 5ARI such as finasteride, dutasteride, izonsteride; SARMs, such as, RU-58642, RU-56279, WS9761 A and B, RU-59063, RU-58841, bexlosteride, LG-2293, L-245976, LG-121071, LG-121091, LG-121104, LGD-2226, LGD-2941, LGD-3303, YM-92088, YM-175735, LGD-1331, BMS-357597, BMS-391197, S-40503, BMS-482404, EM-4283, EM-4977, BMS-564929, BMS-391197, BMS-434588, BMS-487745, BMS-501949, GSK971086, GSK2420A, SA-766, YM-92088, YM-580, LG-123303, LG-123129, PMCol, YM-175735, BMS-591305, BMS-591309, BMS-665139, BMS-665539, CE-590, 116BG33, 154BG31, arcarine, ACP-105; SERMs, such as tamoxifen, 4-hydroxytamoxifen, idoxifene, toremifene, ospemifene, droloxifene, raloxifene, arzoxifene, bazedoxifene, PPT (1,3,5-tris(4-hydroxyphenyl)-4-propyl-1H-pyrazole), diarylpropionitrile (DPN), lasofoxifene, pipendoxifene, EM-800, EM-652, nafoxidine, zindoxifene, tesmilifene, miproxifene phosphate, RU 58,688, EM 139, ICI 164,384, ICI 182,780, clomiphene, MER-25, diethylstilbestrol, coumestrol, genistein, GW5638, LY353581, zuclomiphene, enclomiphene, delmadinone acetate, DPPE, (N, N-diethyl-2-{4-(phenylmethyl)-phenoxy}ethanamine), TSE-424, WAY-070, WAY-292, WAY-818, cyclocommunol, prinaberel, ERB-041, WAY-397, WAY-244, ERB-196, WAY-169122, MF-101, ERb-002, ERB-037, ERB-017, BE-1060, BE-380, BE-381, WAY-358, [18F]FEDNP, LSN-500307, AA-102, CT-101, CT-102, VG-101; GnRH agonists or antagonists, such as, leuprolide, goserelin, triptorelin, alfaprostol, histrelin, detirelix, ganirelix, antide iturelix, cetrorelix, ramorelix, ganirelix, antarelix, teverelix, abarelix, ozarelix, sufugolix, prazarelix, degarelix, NBI-56418, TAK-810, acyline; FSH agonist/antagonist, LH agonist/antagonists, aromatase inhibitors, such as, letrozole, anastrazole, atamestane, fadrozole, minamestane, exemestane, plomestane, liarozole, NKS-01, vorozole, YM-511, finrozole, 4-hydroxyandrostenedione, aminogluethimide, rogletimide; Steroidal or nonsteroidal glucocorticoid receptor ligands, such as, ZK-216348, ZK-243149, ZK-243185, LGD-5552, mifepristone, RPR-106541, ORG-34517, GW-215864X, sesquicillin, CP-472555, CP-394531, A-222977, AL-438, A-216054, A-276575, CP-394531, CP-409069, UGR-07; Steroidal or nonsteroidal progesterone receptor ligands; Steroidal or nonsteroidal AR antagonists such as flutamide, hydroxyflutamide, bicalutamide, nilutamide, hydroxysteroid dehydrogenase inhibitors; PPARα ligand such as bezafibrate, fenofibrate, gemfibrozil; PPARδ ligands such as darglitazone, pioglitazone, rosiglitazone, isaglitazone, rivoglitazone, netoglitazone; Dual acting PPAR ligands, such as naveglitazar, farglitazar, tesaglitazar, ragaglitazar, oxeglitazar, PN-2034, PPARδ; a 17-ketoreductase inhibitors, 3β-ΔHΔ4,6-isomerase inhibitors, 3β-ΔHΔ4,5-isomerase inhibitors, 17,20 desmolase inhibitors, p450c17 inhibitors, p450ssc inhibitors, 17,20-lyase inhibitors, or any combinations thereof.

The invention contemplates, in some embodiments, administration of compositions comprising the individual agents, administered separately and by similar or alternative routes, formulated as appropriately for the route of administration. The invention contemplates, in some embodiments, administration of compositions comprising the individual agents, administered in the same formulation. The invention contemplates, in some embodiments, staggered administration, concurrent administration, of administration of the various agents over a course of time, however, their effects are synergistic in the subject.

It is to be understood that any of the above means, timings, routes, or combinations thereof, of administration of two or more agents is to be considered as being encompassed by the phrase “administered in combination”, as described herein.

In one embodiment, the compound of this invention is administered in combination with an anti-cancer agent. In one embodiment, the anti-cancer agent is a monoclonal antibody. In some embodiments, the monoclonal antibodies are used for diagnosis, monitoring, or treatment of cancer. In one embodiment, monoclonal antibodies react against specific antigens on cancer cells. In one embodiment, the monoclonal antibody acts as a cancer cell receptor antagonist. In one embodiment, monoclonal antibodies enhance the patient's immune response. In one embodiment, monoclonal antibodies act against cell growth factors, thus blocking cancer cell growth. In one embodiment, anti-cancer monoclonal antibodies are conjugated or linked to anti-cancer drugs, radioisotopes, other biologic response modifiers, other toxins, or any combination thereof. In one embodiment, anti-cancer monoclonal antibodies are conjugated or linked to a compound as described hereinabove.

In another embodiment, the present invention includes compounds and compositions in which a compound of the invention is either combined with, or covalently bound to, an agent bound to a targeting agent, such as a monoclonal antibody (e.g., a murine or humanized monoclonal antibody). In one embodiment, the agent bound to a targeting agent is a cytotoxic agent. It will be appreciated that the latter combination may allow the introduction of cytotoxic agents into for example cancer cells with greater specificity. Thus, the active form of the cytotoxic agent (i.e., the free form) will be present only in cells targeted by the antibody. Of course, the compounds of the invention may also be combined with monoclonal antibodies that have therapeutic activity against cancer.

In one embodiment, the compound is administered in combination with a selective tyrosine kinase inhibitor. In some embodiments, the selective tyrosine kinase inhibitor inhibits catalytic sites of cancer promoting receptors thereby inhibiting tumor growth. In one embodiment, a selective tyrosine kinase inhibitor modulates growth factor signaling. In some embodiments, the selective tyrosine kinase inhibitor targets EGFR (ERB B/HER) family members. In one embodiment, the selective tyrosine kinase inhibitor is a BCR-ABL tyrosine kinase inhibitor. In one embodiment, the selective tyrosine kinase inhibitor is an epidermal growth factor receptor tyrosine kinase inhibitor. In one embodiment, the selective tyrosine kinase inhibitor is a vascular endothelial growth factor tyrosine kinase inhibitor. In one embodiment, the selective tyrosine kinase inhibitor is a Platelet Derived Growth Factor (PDGF) inhibitor. In one embodiment, the selective tyrosine kinase inhibitor is an Anaplastic Lymphoma Kinase (ALK) inhibitor.

In one embodiment, the compound is administered in combination with a cancer vaccine. In one embodiment, the cancer vaccine is a therapeutic vaccine thus, treating an existing cancer. In some embodiments, the cancer vaccine is a prophylactic vaccine thus, preventing the development of cancer. In one embodiment, both types of vaccines have the potential to reduce the burden of cancer. In one embodiment, treatment or therapeutic vaccines are administered to cancer patients and are designed to strengthen the body's natural defenses against cancers that have already developed. In one embodiment, therapeutic vaccines may prevent additional growth of existing cancers, prevent the recurrence of treated cancers, or eliminate cancer cells not killed by prior treatments. In some embodiments, prevention or prophylactic vaccines are administered to healthy individuals and are designed to target cancer in individuals who present high risk for the disease. In one embodiment, the cancer vaccine is an antigen/adjuvant vaccine. In one embodiment, the cancer vaccine is a whole cell tumor vaccine. In one embodiment, the cancer vaccine is a dendritic cell vaccine. In one embodiment, the cancer vaccine comprises viral vectors and/or DNA vaccines. In one embodiment, the cancer vaccine is an idiotype vaccine.

In one embodiment, the compound is administered in combination with an anti-cancer chemotherapeutic agent. In one embodiment, the anti-cancer chemotherapeutic agent is an alkylating agent, such as but not limited to cyclophosphamide. In one embodiment, the anti-cancer chemotherapeutic agent is a cytotoxic antibiotic such as but not limited to doxorubicin. In one embodiment, the anti-cancer chemotherapeutic agent is an antimetabolite, such as but not limited to methotrexate. In one embodiment, the anti-cancer chemotherapeutic agent is a vinca alkaloid, such as but not limited to vindesine. In some embodiments, the anti-cancer chemotherapeutic agents include platinum compounds such as but not limited to carboplatin, and taxanes such as docetaxel. In one embodiment, the anti-cancer chemotherapeutic agent is an aromatase inhibitor such as but not limited to anastrazole, exemestane, or letrozole.

In one embodiment, the compound is administered in combination with a Bax activity modulator such as alisol B acetate. In one embodiment, the compound is administered in combination with an angiotensin II receptor blocker such as losartan. In one embodiment, the compound is administered in combination with selenium, green tea cachecins, saw palmetto, lycopene, vitamin D, dietary soy, genistein or isoflavone.

In one embodiment, the compound is administered in combination with antineoplastic agents, such as alkylating agents, antibiotics, hormonal antineoplastics and antimetabolites. Examples of useful alkylating agents include alkyl sulfonates such as busulfan, improsulfan and piposulfan; aziridines, such as a benzodizepa, carboquone, meturedepa and uredepa; ethylenimines and methylmelamines such as altretamine, triethylenemelamine, triethylenephosphoramide, triethylenethiophosphoramide and trimethylolmelamine; nitrogen mustards such as chlorambucil, chlomaphazine, cyclophosphamide, estramustine, iphosphamide, mechlorethamine, mechlorethamine oxide hydrochloride, melphalan, novembichine, phenesterine, prednimustine, trofosfamide, and uracil mustard; nitroso ureas, such as carmustine, chlorozotocin, fotemustine, lomustine, nimustine, ranimustine, dacarbazine, mannomustine, mitobronitol, mitolactol and pipobroman. More such agents will be known to those having skill in the medicinal chemistry and oncology arts.

In some embodiments, other agents suitable for combination with the compounds of this invention include protein synthesis inhibitors such as abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine, modeccin, neomycin, norvaline, pactamycin, paromomycine, puromycin, ricin, α-sarcin, shiga toxin, showdomycin, sparsomycin, spectinomycin, streptomycin, tetracycline, thiostrepton and trimethoprim. Inhibitors of DNA synthesis, including alkylating agents such as dimethyl sulfate, mitomycin C, nitrogen and sulfur mustards, MNNG and NMS; intercalating agents such as acridine dyes, actinomycins, adriamycin, anthracenes, benzopyrene, ethidium bromide, propidium diiodide-intertwining, and agents such as distamycin and netropsin, can also be combined with compounds of the present invention in pharmaceutical compositions. DNA base analogs such as acyclovir, adenine, β-1-D-arabinoside, amethopterin, aminopterin, 2-aminopurine, aphidicolin, 8-azaguanine, azaserine, 6-azauracil, 2′-azido-2′-deoxynucliosides, 5-bromodeoxycytidine, cytosine, β-1-D-arabinoside, diazooxynorleucine, dideoxynucleosides, 5-fluorodeoxycytidine, 5-fluorodeoxyuridine, 5-fluorouracil, hydroxyurea and 6-mercaptopurine also can be used in combination therapies with the compounds of the invention. Topoisomerase inhibitors, such as coumermycin, nalidixic acid, novobiocin and oxolinic acid, inhibitors of cell division, including colcemide, colchicine, vinblastine and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin and streptolydigin also can be combined with the compounds of the invention to provide pharmaceutical compositions.

In one embodiment, the compound is administered in combination with a vaccine for prostate cancer, alisol B acetate, angiotensin II receptor blocker, or others known in the art. In one embodiment, the compound is administered in combination with an agent to decrease prostate (benign or malignant) hypertrophy, such as, for example, selenium, green tea cachecins, saw palmetto, lycopene, vitamin D, dietary soy, genistein and isoflavone food product and others.

In one embodiment, the compound is administered in combination with an immunomodulating agent. In one embodiment, the immunomodulating agent is an immunosuppressive agent. In one embodiment, immunosuppressive agents comprise corticosteroids, cyclosporine, azathioprine, methotrexate, cyclophosphamide, tacrolimus or FK-506, anti-thymocyte globulin, mycophenylate moeftil, or a combination thereof. In one embodiment, the corticosteroid is a glucocorticoid.

In one embodiment, the immunomodulating agent is an immunostimulatory agent. In one embodiment, the immunostimulatory agent is a specific immunostimulator thus, provides antigenic specificity during an immune response, such as a vaccine or any antigen. In one embodiment, the immunostimulatory agent is a non-specific immunostimulator thus, acting irrespective of antigenic specificity to augment immune response of other antigen or stimulate components of the immune system without antigenic specificity. In one embodiment, the non-specific immunostimulator is Freund's complete adjuvant. In one embodiment, the non-specific immunostimulator is Freund's incomplete adjuvant. In one embodiment, the non-specific immunostimulator is a montanide ISA adjuvant. In one embodiment, the non-specific immunostimulator is a Ribi's adjuvant. In one embodiment, the non-specific immunostimulator is a Hunter's TiterMax. In one embodiment, the non-specific immunostimulator is an aluminum salt adjuvant. In one embodiment, the non-specific immunostimulator is a nitrocellulose-adsorbed protein. In one embodiment, the non-specific immunostimulator is a Gerbu Adjuvant.

In one embodiment, the compound is administered in combination with an agent treating the nervous system. In one embodiment, the agent treating the nervous system is an agent treating the autonomic nervous system. In one embodiment, the agent treating the autonomic nervous system is an adrenomimetic drug. In one embodiment, the adrenomimetic drug is a beta-adrenoceptor agonist, alpha-adrenoceptor agonist, or any combination thereof. In one embodiment, the adrenomimetic drug is a catecholamine. In one embodiment, adrenomimetic drugs include but are not limited to isoproterenol, norepinephrine, epinephrine, amphetamine, ephedrine, or dopamine. In one embodiment, the adrenomimetic drug is a directly acting adrenomimetic drug. In some embodiments, directly acting adrenomimetic drugs include but are not limited to phenylephrine, metaraminol, or methoxamine.

In one embodiment, the agent treating the autonomic nervous system is an adrenoceptor antagonist. In one embodiment, the adrenoceptor antagonist is a haloalkylamine, imidazoline, or quinazoline. In one embodiment, haloalkylamines include but are not limited to phenoxybenzamine. In one embodiment, imidazolines include but are not limited to phentolamine or tolazoline. In one embodiment, quinazolines include but are not limited to prazosine, terazosin, doxazosin, or trimazosin. In one embodiment, the adrenoceptor antagonist has a combined alpha and beta blocking activity. In one embodiment, the combined alpha and beta blocking agent is labetalol, bucindolol, carvedilol, or medroxalol

In one embodiment, the agent treating the autonomic nervous system is a cholinomimetic agent. In one embodiment, the cholinomimetic agent is a direct-acting parasympathomimetic drug. In one embodiment, direct-acting parasympathomimetic drugs include but are not limited to methacholine, pilocarpine, carbachol, or bethanechol.

In one embodiment, the agent treating the autonomic nervous system is a cholinesterase inhibitor. In one embodiment, the cholinesterase inhibitor is a quaternary ammonium agent. In one embodiment, quaternary ammonium agents include but are not limited to edrophonium or ambenonium. In one embodiment, the cholinesterase inhibitor is a carbamate such as physostigmine, pyridostigmine, neostigmine, or rivastigmine. In one embodiment, the cholinesterase inhibitor is an organophosphate agent. In one embodiment, the inhibitor targets acetylcholine in the central nervous system such as tacrine, donepezil, or galanthamine.

In one embodiment, the agent treating the autonomic nervous system is a muscarinic blocking agent. In one embodiment, the muscarinic blocking agent is a belladonna alkaloid such as atropine or scopolamine.

In one embodiment, the agent treating the autonomic nervous system is a ganglionic blocking agent. In one embodiment, ganglionic blocking agents include but are not limited to nicotine, trimethaphan, or mecamylamine.

In one embodiment, the agent treating the nervous system is an agent treating the central nervous system. In one embodiment, the agent treating the central nervous system is a local anesthetic agent. In one embodiment, local anesthetic agents include but are not limited to benzocaine, chloroprocaine, cocaine, procaine, bupivacaine, levobupivacaine, lidocaine, mepivacaine, prilocaine, or ropivacaine. In one embodiment, the agent treating the central nervous system is a general anaesthetic agent. In one embodiment, general anesthetic agents include but are not limited to esflurane, sevoflurane, isoflurane, halothane, enflurane, methoxyflurane, xenon, propofol, etomidate, methohexital, midazolam, diazepamor, ketamine, thiopentone/thiopental, or lidocaine/prilocaine.

In one embodiment, the agent treating the central nervous system is an analgesic agent. In some embodiments, analgesic agents include but are not limited to paracetamol or non-steroidal anti-inflammatory agent. In some embodiments, analgesic agents include opiates or morphinomimetics such as morphine, pethidine, oxycodone, hydrocodone, diamorphine, tramadol, or buprenorphine. In some embodiments, a combination of two or more analgesics is desired.

In one embodiment, the agent treating the central nervous system is a muscle relaxant or vasoconstrictor agent. In one embodiment, muscle relaxants include but are not limited to methocarbamol, baclofen, carisoprodol, chlorzoxazone, cyclobenzaprine, dantrolene, metaxalone, orphenadrine, amyl nitrite, pancuronium, tizanidine, clonidine, or gabapentin. In one embodiment, vasoconstrictor agents include but are not limited to antihistamines, adrenalin dimethylarginine, caffeine, cannabis, catecholamines, decongestants, pseudoephedrinses, norepinephrines, tetrahydrozoline, or thromboxane.

In one embodiment, the agent treating the central nervous system is an antiemetic drug. In one embodiment, the antiemetic drug is a 5-HT₃ receptor antagonist such as dolasetron, granisetron, ondansetron, or tropisetron. In one embodiment, the antiemetic drug is a dopamine antagonist such as domperidone droperidol, haloperidol, chlorpromazine, promethazine, or metoclopramide. In one embodiment, the antiemetic drug is an antihistamine such as cyclizine, diphenhydramine, dimenhydrinate, or meclizine. In one embodiment, the antiemetic drug is a cannabinoid such as cannabis or marinol.

In one embodiment, the agent treating the central nervous system is a sedative agent. In one embodiment, the sedative agent is an antidepressant agent such as mirtazapine or trazodone. In one embodiment, the sedative agent is a barbiturate such as secobarbital, pentobarbital, or amobarbital. In one embodiment, the sedative agent is a benzodiazepine such as diazepam, clonazepam, alprazolam, temazepam, chlordiazepoxide, flunitrazepam, lorazepam, or clorazepate. In one embodiment, the sedative agent is an imidazopyridines such as zolpidem or alpidem. In one embodiment, the sedative agent is a pyrazolopyrimidine such as zaleplon. In one embodiment, the sedative agent is an antihistamine such as diphenhydramine, dimenhydrinate, or doxylamine. In one embodiment, the sedative agent is an antipsychotic agent such as ziprasidone, risperidone, quetiapine, clozapine, prochlorperazine, perphenazine, loxapine, trifluoperazine, thiothixene, haloperidol, or fluphenazine. In one embodiment, the sedative agent is an herbal sedative such as valerian plant mandrake, or kava. In some embodiments, the sedative agent is eszopiclone, ramelteon, methaqualone, ethchlorvynol, chloral hydrate, meprobamate, glutethimide, methyprylon, gamma-hydroxybutyrate, ethyl alcohol, methyl trichloride, zopiclone, or diethyl ether.

In one embodiment, the agent treating the central nervous system is a neurodegenerative disorder medication. In one embodiment, the neurodegenerative disorder medication is an acetylcholinesterase inhibitor such as tacrine, donepezil, galanthamine, or rivastigmine. In one embodiment, the neurodegenerative disorder medication is an N-methyl-D-aspartate (NMDA) antagonist such as memantine. In one embodiment, the neurodegenerative disorder medication reduces damage to motor neurons such as riluzole. In one embodiment, the neurodegenerative disorder medication silences the gene that causes the progression of the disease. In one embodiment, the agent treating the central nervous system is an antiepileptic drug (AED). In some embodiments, antiepileptic agents include sodium channel blockers, GABA receptor agonists, GABA reuptake inhibitors, GABA transaminase inhibitor, AEDs with a potential GABA mechanism of action, glutamate blockers, or AEDs with other mechanisms of action. In some embodiments, antiepileptic agents include but are not limited to carbamazepine, fosphenyloin, oxcarbazepine, lamotrigine, zonisamide, clobazam, clonazepam, phenobarbital, primidone, tiagabine, vigabatrin, gabapentin, valproate, felbamate, topiramate, levetiracetam, or pregabalin.

In one embodiment, the compound is administered with an agent treating a wasting disease. In some embodiments, agents treating a wasting disease include but are not limited to corticosteroids, anabolic steroids, cannabinoids, metoclopramide, cisapride, medroxyprogesterone acetate, megestrol acetate, cyproheptadine, hydrazine sulfate, pentoxifylline, thalidomide, anticytokine antibodies, cytokine inhibitors, eicosapentaenoic acid, indomethacin, ibuprofen, melatonin, insulin, growth hormone, clenbuterol, porcine pancreas extract, IGF-1, IGF-1 analogue and secretagogue, myostatin analogue, proteasome inhibitor, testosterone, oxandrolone, Enbrel®, melanocortin 4 receptor agonist, or a combination thereof.

In one embodiment, the agent treating a wasting disease is a ghrelin receptor ligand, growth hormone analogue, or a secretagogue. In some embodiments, ghrelin receptor ligands, growth hormone analogues, or secretagogues include but are not limited to pralmorelin, examorelin, tabimorelin, capimorelin, capromorelin, ipamorelin, EP-01572, EP-1572, or JMV-1843.

In one embodiment, growth promoting agents such as but not limited to TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890 are utilized as agents treating a wasting disease.

In other embodiments, agents treating a wasting disease may comprise growth hormone secretagogues such as GHRP-6, GHRP-1 (as described in U.S. Pat. No. 4,411,890 and publications WO 89/07110 and WO 89/07111), GHRP-2 (as described in WO 93/04081), NN703 (Novo Nordisk), LY444711 (Lilly), MK-677 (Merck), CP424391 (Pfizer) and B-HT920, or, in other embodiments, with growth hormone releasing factor and its analogs or growth hormone and its analogs, or with alpha-adrenergic agonists, such as clonidine or serotinin 5-HTD agonists, such as sumatriptan, or agents which inhibit somatostatin or its release, such as physostigmine and pyridostigmine. In some embodiments, agents treating a wasting disease may comprise parathyroid hormone, PTH(1-34) or bisphosphonates, such as MK-217 (alendronate). In other embodiments, agents treating wasting disease may further comprise estrogen, a selective estrogen receptor modulator, such as tamoxifen or raloxifene, or other androgen receptor modulators, such as those disclosed in Edwards, J. P. et al., Bio. Med. Chem. Let., 9, 1003-1008 (1999) and Hamann, L. G. et al., J. Med. Chem., 42, 210-212 (1999). In some embodiments, agents treating a wasting disease may further comprise a progesterone receptor agonists (“PRA”), such as levonorgestrel, medroxyprogesterone acetate (MPA). In some embodiments, agents treating a wasting disease may include nutritional supplements, such as those described in U.S. Pat. No. 5,179,080, which, in other embodiments are in combination with whey protein or casein, amino acids (such as leucine, branched amino acids and hydroxymethylbutyrate), triglycerides, vitamins (e.g., A, B6, B 12, folate, C, D and E), minerals (e.g., selenium, magnesium, zinc, chromium, calcium and potassium), carnitine, lipoic acid, creatinine, R-hydroxy-β-methylbutyrate (Juven) and coenzyme Q. In one embodiment, agents treating a wasting disease may further comprise antiresorptive agents, vitamin D analogues, elemental calcium and calcium supplements, cathepsin K inhibitors, MMP inhibitors, vitronectin receptor antagonists, Src SH2 antagonists, vacular-H⁺-ATPase inhibitors, ipriflavone, fluoride, tibolone, prostanoids, 17-beta hydroxysteroid dehydrogenase inhibitors and Src kinase inhibitors.

In one embodiment, the compound of this invention is administered with an inhibitor of an enzyme involved in the androgen biosynthetic pathway. In some embodiments, inhibitors of enzymes involved in the androgen biosynthetic pathway include but are not limited to 17-ketoreductase inhibitor, 3-H4,6-isomerase inhibitor, 3β-ΔHΔ-4,5-isomerase inhibitors, 17,20 desmolase inhibitor, p450c17 inhibitor, p450ssc inhibitor, or 17,20-lyase inhibitor.

In some embodiments, any of the compositions of this invention will comprise a compound of formula I-V(a), in any form or embodiment as described herein. In some embodiments, any of the compositions of this invention will consist of a compound of formula I-V(a), in any form or embodiment as described herein. In some embodiments, of the compositions of this invention will consist essentially of a compound of formula I-V(a), in any form or embodiment as described herein. In some embodiments, the term “comprise” refers to the inclusion of the indicated active agent, such as the compound of formula I-V(a), as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

In one embodiment, the present invention provides combined preparations. In one embodiment, the term “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

It is to be understood that this invention is directed to compositions and combined therapies as described herein, for any disease, disorder or condition, as appropriate, as will be appreciated by one skilled in the art. Certain applications of such compositions and combined therapies have been described hereinabove, for specific diseases, disorders and conditions, representing embodiments of this invention, and methods of treating such diseases, disorders and conditions in a subject by administering a compound as herein described, alone or as part of the combined therapy or using the compositions of this invention represent additional embodiments of this invention.

### Biological Activity of Compound of the Invention

In one embodiment, this invention is directed to a method of treating, suppressing, reducing the severity, reducing the risk of developing or inhibiting cancer comprising administering a compound of this invention to a subject suffering from cancer under conditions effective to treat the cancer.

The compounds of the present invention are useful in the treatment, reducing the severity, reducing the risk, or inhibition of cancer, metastatic cancer, drug resistant tumors, drug resistant cancer and various forms of cancer. In a preferred embodiment the cancer is prostate cancer, breast cancer, ovarian cancer, skin cancer (e.g., melanoma), bladder cancer, lung cancer, colon cancer, leukemia, lymphoma, head and neck, pancreatic, esophageal, renal cancer or CNS cancer (e.g., glioma, glioblastoma). Treatment of these different cancers is supported by the Examples herein. Moreover, based upon their believed mode of action as tubulin inhibitors, it is believed that other forms of cancer will likewise be treatable or preventable upon administration of the compounds or compositions of the present invention to a patient. Preferred compounds of the present invention are selectively disruptive to cancer cells, causing ablation of cancer cells but preferably not normal cells. Significantly, harm to normal cells is minimized because the cancer cells are susceptible to disruption at much lower concentrations of the compounds of the present invention.

In some embodiments, this invention provides for the use of a compound as herein described, or its isomer, pharmaceutically acceptable salt, pharmaceutical product, tautomer, N-oxide, hydrate or any combination thereof, for treating, suppressing, reducing the severity, reducing the risk, or inhibiting cancer in a subject. In another embodiment, the cancer is adrenocortical carcinoma, anal cancer, bladder cancer, brain tumor, brain stem, breast cancer, glioma, cerebellar astrocytoma, cerebral astrocytoma, ependymoma, medulloblastoma, supratentorial primitive neuroectodermal, pineal tumors, hypothalamic glioma, carcinoid tumor, carcinoma, cervical cancer, colon cancer, central nervous system (CNS) cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian cancer, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor, or any combination thereof. In another embodiment the subject has been previously treated with chemotherapy, radiotherapy or biological therapy.

In some embodiments, this invention provides for the use of a compound as herein described, or its isomer, pharmaceutically acceptable salt, pharmaceutical product, tautomer, N-oxide, hydrate or any combination thereof, for treating, suppressing, reducing the severity, reducing the risk, or inhibiting a metastatic cancer in a subject. In another embodiment, the cancer is adrenocortical carcinoma, anal cancer, bladder cancer, brain tumor, brain stem, breast cancer, glioma, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, central nervous system (CNS) cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian cancer, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor, or any combination thereof.

In one embodiment “metastatic cancer” refers to a cancer that spread (metastasized) from its original site to another area of the body. Virtually all cancers have the potential to spread. Whether metastases develop depends on the complex interaction of many tumor cell factors, including the type of cancer, the degree of maturity (differentiation) of the tumor cells, the location and how long the cancer has been present, as well as other incompletely understood factors. Metastases spread in three ways—by local extension from the tumor to the surrounding tissues, through the bloodstream to distant sites or through the lymphatic system to neighboring or distant lymph nodes. Each kind of cancer may have a typical route of spread. The tumor is called by the primary site (ex. breast cancer that has spread to the brain is called metastatic breast cancer to the brain).

In some embodiments, this invention provides for the use of a compound as herein described, or its isomer, pharmaceutically acceptable salt, pharmaceutical product, tautomer, N-oxide, hydrate or any combination thereof, for treating, suppressing, reducing the severity, reducing the risk, or inhibiting a drug-resistant cancer or resistant cancer in a subject. In another embodiment, the cancer is adrenocortical carcinoma, anal cancer, bladder cancer, brain tumor, brain stem, breast cancer, glioma, cerebellar astrocytoma, cerebral astrocytoma, ependymoma, medulloblastoma, supratentorial primitive neuroectodermal, pineal tumors, hypothalamic glioma, breast cancer, carcinoid tumor, carcinoma, cervical cancer, colon cancer, central nervous system (CNS) cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metasatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian cancer, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor, or any combination thereof.

Drug resistance is the major cause of cancer chemotherapy failure. One major contributor to multidrug resistance is overexpression of P-glycoprotein (P-gp). This protein is a clinically important transporter protein belonging to the ATP-binding cassette family of cell membrane transporters. It can pump substrates including anticancer drugs out of tumor cells through an ATP-dependent mechanism.

The term “drug-resistant cancer” refers to cancer cells that acquire resistance to chemotherapy. “Multiple drug resistance” or “Multidrug resistance” (MDR) refers to a condition enabling a disease-causing organism to resist distinct drugs or chemicals of a wide variety of structure and function targeted at eradicating the organism. Organisms that display multidrug resistance can be pathologic cells, including neoplastic (tumor) cells. Cancer cells can acquire resistance to chemotherapy by a range of mechanisms, including the mutation or overexpression of the drug target, inactivation of the drug, or elimination of the drug from the cell (increased efflux of drug by P-glycoprotein, multidrug resistance-associated protein, lung resistance-related protein, breast cancer resistance protein and reproductive cancer resistance protein). Tumors that recur after an initial response to chemotherapy may be resistant to multiple drugs (they are multidrug resistant). In the conventional view of drug resistance, one or several cells in the tumor population acquire genetic changes that confer drug resistance. Accordingly, the reasons for drug resistance, inter alia, are: a) some of the cells that are not killed by the chemotherapy mutate (change) and become resistant to the drug. Once they multiply, there may be more resistant cells than cells that are sensitive to the chemotherapy; b) gene amplification. A cancer cell may produce hundreds of copies of a particular gene. This gene triggers an overproduction of protein that renders the anticancer drug ineffective; c) cancer cells may pump the drug out of the cell as fast as it is going into the cells using a molecule called p-glycoprotein; d) cancer cells may stop taking in the drugs because the protein that transports the drug across the cell wall stops working; e) the cancer cells may learn how to repair the DNA breaks caused by some anti-cancer drugs; f) cancer cells may develop a mechanism that inactivates the drug. One major contributor to multidrug resistance is overexpression of P-glycoprotein (P-gp). This protein is a clinically important transporter protein belonging to the ATP-binding cassette family of cell membrane transporters. It can pump substrates including anticancer drugs out of tumor cells through an ATP-dependent mechanism. Thus, the resistance to anticancer agents used in chemotherapy is the main cause of treatment failure in malignant disorders, provoking tumors to become resistant. Drug resistance is the major cause of cancer chemotherapy failure, and P-gp overexpression is a major contributor to multidrug resistance cancer.

In one embodiment “resistant cancer” refers to drug-resistant cancer as described herein above. In another embodiment “resistant cancer” refers to cancer cells that acquire resistance to any treatment such as chemotherapy, radiotherapy or biological therapy.

The compounds of this invention may be useful, in some embodiments, for treatment, prevention, prevention of the reccurence, suppression, halting, suppressing, reducing the severity, reducing the incidence, causing the regression of, inhibition, or reduction of the risk of developing cancer, including inter-alia (a) prostate cancer, (b) advanced prostate cancer, (c) castration-resistant prostate cancer (d) drug resistant prostate cancer (e) metastatic prostate cancer; (f) breast cancer; (g) melanoma, (h) drug resistant melanoma (i) metastatic melanoma; (j) colon cancer; (k) bladder cancer; and/or other clinical therapeutic and/or diagnostic areas, including any embodiment of what is encompassed by the term “treating” as described herein.

The term “castration-resistant prostate cancer” (CRPC) refers to an advanced prostate cancer which developed despite ongoing Androgen Deprivation Therapy (ADT) and/or surgical castration. In one embodiment, CRPC refers to prostate cancer which is considered hormone refractory, androgen independent or castration resistant. In another embodiment, CRPC refers to prostate cancer that continues to progress or worsen or adversely affect the health of the patient despite prior surgical castration, continued treatment with gonadotropin releasing hormone agonists (e.g., leuprolide) or antagonists (degarelix), antiandrogens (e.g., bicalutamide, flutamide, MDV3100, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa®)) or other prostate cancer therapies (e.g., vaccines (sipuleucel-T (Provenge®), GVAX, etc.), herbal (PC-SPES) and lyase inhibitor (abiraterone)) as evidenced by increasing or higher serum levels of prostate specific antigen (PSA), metastasis, bone metastasis, pain, lymph node involvement, increasing size or serum markers for tumor growth, worsening diagnostic markers of prognosis, or patient condition.

In one embodiment, this invention is directed to treating, suppressing, reducing the severity, reducing the risk, or inhibiting cancer in a subject, wherein the subject has been previously treated with chemotherapy, radiotherapy or biological therapy.

In one embodiment, this invention is directed to treating, halting, suppressing, reducing the severity, reducing the incidence of, reducing the risk, causing the regression of, inhibiting, preventing or preventing the reccurence of treating, suppressing, reducing the severity, reducing the risk, or inhibiting prostate cancer in a subject, wherein the subject has been previously treated with chemotherapy, radiotherapy or biological therapy.

In one embodiment, this invention is directed to treating, halting, suppressing, reducing the severity, reducing the incidence of, reducing the risk, causing the regression of, inhibiting, preventing or preventing the reccurence of treating, suppressing, reducing the severity, reducing the risk, or inhibiting breast cancer in a subject, wherein the subject has been previously treated with chemotherapy, radiotherapy or biological therapy.

In one embodiment, this invention provides methods for: (a) treating, halting, suppressing, reducing the severity, reducing the incidence of, reducing the risk, causing the regression of, or inhibiting, preventing or preventing the recurrence prostate cancer; (b) treating, suppressing, reducing the severity, reducing the risk, or inhibiting drug resistant prostate cancer; (c) treating, suppressing, reducing the severity, reducing the risk, or inhibiting castration-resistant prostate cancer; (d) treating, suppressing, reducing the severity, reducing the risk, or inhibiting metastatic prostate cancer; (e) treating, suppressing, reducing the severity, reducing the risk, or inhibiting advanced prostate cancer; (f) treating, suppressing, reducing the severity, reducing the risk, or inhibiting melanoma; (g) treating, suppressing, reducing the severity, reducing the risk, or inhibiting metastatic melanoma; (h) treating, suppressing, reducing the severity, reducing the risk, or inhibiting drug resistant melanoma; (i) treating, suppressing, reducing the severity, reducing the risk, or inhibiting breast cancer; (j) treating, suppressing, reducing the severity, reducing the risk, or inhibiting colon cancer; (k) treating, suppressing, reducing the severity, reducing the risk, or inhibiting bladder cancer in a subject; wherein the subject has been previously treated with chemotherapy, radiotherapy, or biological therapy; comprising the step of administering to said subject a compound of this invention and/or an isomer, pharmaceutically acceptable salt, pharmaceutical product, tautomer, hydrate, N-oxide, or crystal of said compound, or any combination thereof. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In one embodiment “Chemotherapy” refers to chemical treatment for cancer such as drugs that kill cancer cells directly. Such drugs are referred as “anti-cancer” drugs or “antineoplastics.” Today's therapy uses more than 100 drugs to treat cancer or to cure a specific cancer. Chemotherapy is used to control tumor growth when cure is not possible; to shrink tumors before surgery or radiation therapy; to relieve symptoms (such as pain); and to destroy microscopic cancer cells that may be present after the known tumor is removed by surgery (called adjuvant therapy). Adjuvant therapy is given to prevent a possible cancer reoccurrence.

In one embodiment, “Radiotherapy” refers to high energy x-rays and similar rays (such as electrons) to treat disease. Many people with cancer will have radiotherapy as part of their treatment. This can be given either as external radiotherapy from outside the body using x-rays or from within the body as internal radiotherapy. Radiotherapy works by destroying the cancer cells in the treated area. Although normal cells can also be damaged by the radiotherapy, they can usually repair themselves. Radiotherapy treatment can cure some cancers and can also reduce the chance of a cancer coming back after surgery. It may be used to reduce cancer symptoms.

In one embodiment “Biological therapy” refers to substances that occur naturally in the body to destroy cancer cells. There are several types of treatment including: monoclonal antibodies, cancer growth inhibitors, vaccines and gene therapy. Biological therapy is also known as immunotherapy.

In other embodiments, this invention provides methods of inhibiting tubulin polymerization in a cell associated with a cell proliferative disease comprising contacting the cell associated with the cell proliferative disease with a pharmacologically effective amount of at least one compound described herein. In one embodiment, the cell proliferative disease may be cancer. Representative examples of cancers include prostate cancer, melanoma, colon cancer, bladder cancer or breast cancer.

The compounds provided herein may be useful as therapeutics to inhibit growth of abnormally proliferating cells in a cell proliferative disease by inhibiting tubulin or tubulin polymerization in the cell while circumventing ATP binding cassette transporter mediated multi-drug resistance. It is contemplated that contacting the abnormally proliferating cells with this compound is effective to induce apoptosis and/or cell cycle arrest. Thus, the compounds of the present invention may be useful in treating cancers in a subject. In some embodiments, the subject is a mammal. In other embodiments, the subject is a human. In other embodiments, the subject is a male or female. Examples of cancers may include, but are not limited to, prostate cancer, melanoma, colon cancer, bladder cancer or breast cancer.

In one embodiment, this invention provides a method for inhibiting tubulin polymerization in a subject's cell that is associated with a cell proliferative disease comprising the step of administering to said subject a compound of this invention, or its isomer, pharmaceutically acceptable salt, pharmaceutical product, tautomer, hydrate, N-oxide, or any combination thereof, or a composition comprising the same in an amount effective to inhibit tubulin polymerization in said proliferative disease cell. In one embodiment, the proliferative disease cell may be cancer. Representative examples of cancers include prostate cancer, melanoma, colon cancer, bladder cancer or breast cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides methods of treating, halting, suppressing, reducing the severity of, delaying the progression of, reducing the incidence of, reducing the risk of, causing the regression of, inhibiting, preventing or preventing the reccurence of cancer in a subject comprising the step of administering to said subject a compound of this invention, or its isomer, pharmaceutically acceptable salt, pharmaceutical product, tautomer, hydrate, N-oxide, or any combination thereof, or a composition comprising the same in an amount effective to inhibit the growth of cancer cells thereby, halting, suppressing, reducing the severity of, delaying the progression of, reducing the incidence of, reducing the risk of, causing the regression of, inhibiting, preventing or preventing the reccurence of cancer in said subject. Representative examples of cancers include prostate cancer, melanoma, colon cancer, bladder cancer or breast cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

According to another embodiment, the patient to be treated is characterized by the presence of a cancerous condition, and the administering of the compound is effective either to cause regression of the cancerous condition or to inhibit growth of the cancerous condition, i.e., stopping its growth altogether or reducing its rate of growth. This preferably occurs by destroying cancer cells, regardless of their location in the patient body. That is, whether the cancer cells are located at a primary tumor site or whether the cancer cells have metastasized and created secondary tumors within the patient body.

In one embodiment, this invention provides a method of treating, halting, suppressing, reducing the severity, reducing the incidence of, reducing the risk, causing the regression of, or inhibiting a drug-resistant cancerous tumor or tumors in a subject comprising the step of administering to said subject a compound of this invention, in an amount effective to treat, halt, suppress, reduce the severity, reduce the incidence of, reduce the risk of, cause the regressions of, or inhibit said drug-resistant tumor. In one embodiment, the tumor is prostate cancer tumor. In another embodiment, the tumor is breast cancer tumor. In another embodiment, the tumor is colon cancer tumor. In another embodiment, the tumor is bladder cancer tumor. In another embodiment, the tumor is melanoma tumor. In another embodiment, the tumor is ovarian cancer tumor. In another embodiment, the tumor is a multidrug resistant (MDR) tumor. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In one embodiment, this invention provides a method of treating a subject suffering from prostate cancer, advanced prostate cancer, metastatic prostate cancer, resistant prostate cancer, castration-resistant prostate cancer (CRPC) or multidrug-resistant prostate cancer comprising the step of administering to said subject a compound of this invention, or its isomer, pharmaceutically acceptable salt, pharmaceutical product, tautomer, hydrate, N-oxide, or any combination thereof, or a composition comprising the same in an amount effective to treat prostate cancer, advanced prostate cancer, metastatic prostate cancer, resistant prostate cancer, castration-resistant prostate cancer (CRPC) or multidrug-resistant prostate cancer in said subject. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In one embodiment, this invention provides a method for halting, suppressing, reducing the severity of, delaying the progression of, reducing the incidence of, reducing the risk of, causing the regression of, inhibiting, preventing or preventing the reccurence of prostate cancer, advanced prostate cancer, metastatic prostate cancer, resistant prostate cancer, castration-resistant prostate cancer (CRPC) or multidrug-resistant prostate cancer in a subject, comprising administering to said subject a compound of this invention and/or its isomer, pharmaceutically acceptable salt, pharmaceutical product, tautomer, hydrate, N-oxide, or any combination thereof, or a composition comprising the same. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In one embodiment, this invention provides a method for treating, halting, suppressing, reducing the severity, delaying the progression of, reducing the incidence of, reducing the risk of, causing the regression of, inhibiting, preventing or preventing the reccurence of melanoma, metastatic melanoma, resistant melanoma or multidrug-resistant melanoma in a subject, comprising administering to the subject a compound of this invention and/or its isomer, pharmaceutically acceptable salt, pharmaceutical product, tautomer, hydrate, N-oxide, or any combination thereof. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In one embodiment, this invention provides a method of treating a subject suffering from breast cancer, metastatic breast cancer, resistant breast cancer or multidrug-resistant breast cancer comprising the step of administering to said subject a compound of this invention, or its isomer, pharmaceutically acceptable salt, pharmaceutical product, tautomer, hydrate, N-oxide, or any combination thereof, or a composition comprising the same. In another embodiment, the subject is a male or female. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In one embodiment, this invention provides a method of halting, suppressing, reducing the severity, delaying the progression of, reducing the incidence of, reducing the risk of, causing the regression of, inhibiting, preventing or preventing the reccurence of breast cancer, metastatic breast cancer, resistant breast cancer or drug-resistant breast cancer in a subject comprising the step of administering to said subject a compound of this invention or its isomer, pharmaceutically acceptable salt, pharmaceutical product, tautomer, hydrate, N-oxide, or any combination thereof, or a composition comprising the same. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In one embodiment, this invention is directed to a method of destroying a cancerous cell comprising: providing a compound of this invention and contacting the cancerous cell with the compound under conditions effective to destroy the contacted cancerous cell. According to various embodiments of destroying the cancerous cells, the cells to be destroyed can be located either in vivo or ex vivo (i.e., in culture). In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148. In another embodiment, the cancer is prostate cancer. In another embodiment, the cancer is breast cancer. In another embodiment, the cancer is melanoma. In another embodiment, the cancer is colon cancer. In another embodiment, the cancer is bladder cancer.

In another embodiment, this invention provides for the use of a compound as herein described, or its isomer, pharmaceutically acceptable salt, pharmaceutical product, tautomer, hydrate, N-oxide, or any combination thereof, for treating, suppressing, reducing the severity, reducing the risk, delaying the progression, or inhibiting ovarian cancer, metastatic ovarian cancer, resistant ovarian cancer or multidrug-resistant ovarian cancer in a subject. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or its isomer, pharmaceutically acceptable salt, pharmaceutical product, tautomer, hydrate, N-oxide, or any combination thereof, for treating, suppressing, reducing the severity, reducing the risk, delaying the progression, or inhibiting bladder cancer, metastatic bladder cancer, resistant bladder cancer or multidrug-resistant bladder cancer in a subject. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting lung cancer, metastatic lung cancer, resistant lung cancer or multidrug-resistant lung cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting non-small cell lung cancer, metastatic non-small cell lung cancer, resistant non-small cell lung cancer or multidrug-resistant non-small cell lung cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting colon cancer, metastatic colon lung cancer, resistant colon cancer or multidrug-resistant colon cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting of leukemia, metastatic leukemia, resistant leukemia or multidrug-resistant leukemia. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting lymphoma, metastatic lymphoma, resistant lymphoma or multidrug-resistant lymphoma. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting head and neck cancer, metastatic head and neck cancer, resistant head and neck cancer or multidrug-resistant head and neck cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting of pancreatic cancer, metastatic pancreatic cancer, resistant pancreatic cancer or multidrug-resistant pancreatic cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting esophageal cancer, metastatic esophageal cancer, resistant esophageal cancer or multidrug-resistant esophageal cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting renal cancer, metastatic renal cancer, resistant renal cancer or multidrug-resistant renal cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

In another embodiment, this invention provides for the use of a compound as herein described, or isomer, pharmaceutically acceptable salt, pharmaceutical product, tautomer, hydrate, N-oxide, or any combination thereof, for treating, suppressing, reducing the severity, reducing the risk, delaying the progression, or inhibiting CNS cancer, metastatic CNS cancer, resistant CNS cancer or multidrug-resistant CNS cancer. In another embodiment, the compound is compound 68. In another embodiment, the compound is compound 13. In another embodiment, the compound is compound 81. In another embodiment, the compound is compound 82. In another embodiment, the compound is compound 83. In another embodiment, the compound is compound 84. In another embodiment, the compound is compound 80A. In another embodiment, the compound is compound 90. In another embodiment, the compound is compound 125. In another embodiment, the compound is compound 126. In another embodiment, the compound is compound 115. In another embodiment, the compound is compound 117. In another embodiment, the compound is compound 148.

A still further aspect of the present invention relates to a method of treating or preventing a cancerous condition that includes: providing a compound of the present invention and then administering an effective amount of the compound to a patient in a manner effective to treat or prevent a cancerous condition. When the compounds or pharmaceutical compositions of the present invention are administered to treat, suppress, reduce the severity, reduce the risk, or inhibit a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, immunotherapy, chemotherapy, surgical intervention, and combinations thereof.

According to one embodiment, the patient to be treated is characterized by the presence of a precancerous condition, and the administering of the compound is effective to prevent development of the precancerous condition into the cancerous condition. This can occur by destroying the precancerous cell prior to or concurrent with its further development into a cancerous state.

In some embodiments, the present invention provides a method for treating, reducing the incidence, delaying the onset or progression, or reducing and/or abrogating the symptoms associated with a combination of diseases and/or disorders in a subject as described hereinabove, comprising administering to said subject an effective amount of a compound according to this invention or its isomer, pharmaceutically acceptable salt, pharmaceutical product, N-oxide, hydrate or any combination thereof.

As used herein, subject or patient refers to any mammalian patient, including without limitation, humans and other primates, dogs, cats, horses, cows, sheep, pigs, rats, mice, and other rodents.

In one embodiment, the methods of this invention are useful a subject, which is a human. In another embodiment, the subject is a mammal. In another embodiment the subject is an animal. In another embodiment the subject is an invertebrate. In another embodiment the subject is a vertebrate.

In one embodiment, the subject is male. In another embodiment, the subject is female. In some embodiments, while the methods as described herein may be useful for treating either males or females.

In some embodiments, while the methods as described herein may be useful for treating either males or females, males may respond more advantageously to administration of certain compounds, for certain methods, as described herein.

When administering the compounds of the present invention, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

The compounds of the present invention are useful in the treatment or prevention of various forms of cancer, particularly prostate cancer, breast cancer, ovarian, skin cancer (e.g., melanoma), bladder cancer, lung cancer, colon cancer, leukemia, renal cancer, and CNS cancer (e.g., glioma, glioblastoma). Treatment of these different cancers is supported by the Examples herein. Moreover, based upon their believed mode of action as tubulin inhibitors, it is believed that other forms of cancer will likewise be treatable or preventable upon administration of the compounds or compositions of the present invention to a patient. Preferred compounds of the present invention are selectively disruptive to cancer cells, causing ablation of cancer cells but preferably not normal cells. Significantly, harm to normal cells is minimized because the cancer cells are susceptible to disruption at much lower concentrations of the compounds of the present invention.

A person skilled in the art would readily recognize that changes in the antineoplastic therapy according to the methods provided herein, utilizing the compositions provided herein may be conducted as a function of, or adjusted or varied as a function of, inter alia, the severity of the underlying disease, the source of the underlying disease, the extent of the patients' pain and source of the patients' pain, as well as the stage of the disease. The therapeutic changes may include in certain embodiments, changes in the route of administration (e.g. intracavitarily, intraarterially, intratumorally etc.), forms of the compositions administered (e.g. tablets, elixirs, suspensions etc.), changes in dosage and the like. Each of these changes are well recognized in the art and are encompassed by the embodiments provided herein.

In one embodiment, the methods of the present invention comprise administering a compound of this invention as the sole active ingredient. However, also encompassed within the scope of the present invention are methods which comprise administering the compounds in combination with one or more therapeutic agents. These agents include, but are not limited to: LHRH analogs, reversible antiandrogens, antiestrogens, anticancer drugs, 5-alpha reductase inhibitors, aromatase inhibitors, progestins, agents acting through other nuclear hormone receptors, selective estrogen receptor modulators (SERM), progesterone, estrogen, PDE5 inhibitors, apomorphine, bisphosphonate, and one or more SARMs.

In one embodiment, the methods of the present invention comprise administering the compound in combination with an LHRH analog. In another embodiment, the methods of the present invention comprise administering the compound, in combination with a reversible antiandrogen. In another embodiment, the methods of the present invention comprise administering the compound, in combination with an antiestrogen. In another embodiment, the methods of the present invention comprise administering the compound, in combination with an anticancer drug. In another embodiment, the methods of the present invention comprise administering the compound, in combination with a 5-alpha reductase inhibitor. In another embodiment, the methods of the present invention comprise administering the compound, in combination with an aromatase inhibitor. In another embodiment, the methods of the present invention comprise administering the compound, in combination with a progestin. In another embodiment, the methods of the present invention comprise administering the compound, in combination with an agent acting through other nuclear hormone receptors. In another embodiment, the methods of the present invention comprise administering the compound, in combination with a selective estrogen receptor modulators (SERM). In another embodiment, the methods of the present invention comprise administering the compound, in combination with a progesterone. In another embodiment, the methods of the present invention comprise administering the compound, in combination with an estrogen. In another embodiment, the methods of the present invention comprise administering the compound, in combination with a PDE5 inhibitor. In another embodiment, the methods of the present invention comprise administering the compound, in combination with apomorphine. In another embodiment, the methods of the present invention comprise administering the compound, in combination with a bisphosphonate. In another embodiment, the methods of the present invention comprise administering the compound, in combination with one or more SARMs.

In some embodiments, the methods of the present invention comprise combined preparations comprising the compound and an agent as described hereinabove. In some embodiments, the combined preparations can be varied, e.g., in order to cope with the needs of a patient subpopulation to be treated or the needs of the single patient which different needs can be due to the particular disease, severity of the disease, age, sex, or body weight as can be readily determined by a person skilled in the art. In some embodiments, the methods of the present invention comprise personalized medicine methods which treat the needs of a single patient. In one embodiment, different needs can be due to the particular disease, severity of the disease, the overall medical state of a patient, or the age of the patient. In some embodiments, personalized medicine is the application of genomic data to better target the delivery of medical interventions. Methods of personalized medicine, in some embodiments, serve as a tool in the discovery and clinical testing of new products of the present invention. In one embodiment, personalized medicine involves the application of clinically useful diagnostic tools that may help determine a patient's predisposition to a particular disease or condition. In some embodiments, personalized medicine is a comprehensive approach utilizing molecular analysis of both patients and healthy individuals to guide decisions throughout all stages of the discovery and development of pharmaceuticals and diagnostics; and applying this knowledge in clinical practice for a more efficient delivery of accurate and quality healthcare through improved prevention, diagnosis, treatment, and monitoring methods.

It is to be understood that any method of this invention, as herein described, encompasses the administration of a compound as herein described, or a composition comprising the same, to the subject, in order to treat the indicated disease, disorder or condition. The methods as herein described each and/or all may further comprise administration of an additional therapeutic agent as herein described, and as will be appreciated by one skilled in the art.

It is to be understood that any use of any of the compounds as herein described may be used in the treatment of any disease, disorder or condition as described herein, and represents an embodiment of this invention.

The following examples are presented in order to more fully illustrate the preferred embodiments of the invention. They should in no way, however, be construed as limiting the broad scope of the invention.

### EXAMPLES

### Example 1

### Synthesis of Compounds

### Materials and Methods

### General

All reagents were purchased from Sigma-Aldrich Chemical Co., Fisher Scientific (Pittsburgh, Pa.), Alfa Aesar (Ward Hill, Mass.), and AK Scientific (Mountain View, Calif.) and were used without further purification. The solvents for moisture-sensitive reactions were freshly distilled, and the reactions were carried out under an argon atmosphere. Routine thin layer chromatography (TLC) was performed on aluminum-backed Uniplates (Analtech, Newark, Del.). Melting points were measured with Fisher-Johns melting point apparatus (uncorrected). NMR spectra were obtained on a Varian Inova-500 spectrometer or a Bruker AX 300 (Billerica, Mass.) spectrometer. Chemical shifts are reported as parts per million (ppm) relative to TMS in CDCl₃. Mass spectra were collected on a Bruker ESQUIRE electrospray/ion trap instrument in positive and negative ion modes. The purity of the final compounds was examined via RP-HPLC on a Waters 2695 HPLC system installed with a Photodiode Array Detector. Two RP-HPLC methods were conducted using a Supelco Ascentis™ 5 μM RP-Amide column (250×4.6 mm) at ambient temperature, and a flow rate of 0.7 mL/min. HPLC1: Gradient: Solvent A (water) and Solvent B (methanol): 0-15 min 40-100% B (linear gradient), 15-25 min 100% B. HPLC2: Gradient: Solvent A (water) and Solvent B (methanol): 0-5 min 10-40% B (linear gradient), 5-15 min 40-100% B, 15-30 min 100% B. UV detection at 254 nm.

Purities of the compounds were established by careful integration of areas for all peaks detected and are reported for each compound in the following section.

General Synthetic Scheme

Known synthetic methods are used to synthesize the compounds 10, 11, and 13 as shown in FIG. 1. As shown in the synthetic scheme in FIG. 1, target diindoles 10, 11 and 13 bridged via methylphenyl linkers were prepared by removing protecting group benzenesulfonyl under reflux of ethanolic NaOH solution from corresponding precursor compounds 9, 8 and 12 using a general procedure described below. Intermediate compound 8 is key to the subsequent synthesis of compounds 10, 11 and 13. Compound 8 is synthesized from the coupling of protected indole 1 with protected indole benzaldehyde compound 5 in the presence of lithium diisopropyl amide (LDA) as a 94% yield. Compound 5 may be synthesized using two different Suzuki coupling pathways, path A and path B.

For path A, the lithiation of protected indole 1 by LDA to yield indole 2 followed by bromination with cyanogen bromide (BrCN) produced bromoindole 3. The synthesized bromoindole 3 was coupled with aldehydrophenylboric acid 4 to yield compound 5.

For path B, compound 5 was prepared using commercially available iodophenylaldehyde 6 and protected indole boric acid 7.

Using triethylsilane and trifluoroacetic acid (TFA), the phenylmethanol linker in compound 8 was additively reduced to phenylmethylene in compound 9 at rt as a 67% yield. In this reduction, triphenylsilane was used as another silylating agent had poor yield because of resistance for its bulky group. Methylphenyl-linked diindole compound 10 was afforded from protected diindole 9 by the general procedure. By treating compound 8 with sodium hydroxide (10 eq.) under reflux ethanol for 20 h, the free methanol-linked diindole 11 was produced.

By oxidizing the phenylmethanol linker in compound 8 with pyridinium dichromate (PDC) in dimethylformamide (DMF), the protected phenylmethanone linked compound 12 was synthesized as a 73% yield. Phenylmethanone-linked compound 13 was synthesized by the general procedure from compound 12 as an 83% yield.

### Synthesis of 2-bromo-1-(benzenesulfonyl)indole (3)

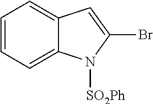

Compound 3 was prepared by Ketcha's method (Ketcha et al. J. Org. Chem. 1989, 54: 4350-4356). Calculated mass 334.96, [M−H] 334.1. Anal. calc. for C₁₄H₁₀BrNO₂S; C, H, N.

### Synthesis of 3-(1-(benzenesulfonyl-1H-indol-2-yl)benzaldehyde (5)

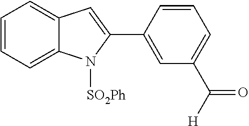

Compound 5 was produced by Suzuki's coupling using path A or path B. Path A and path B utilize the same procedure of coupling an organoboronic acid with an aryl halide, but path A uses the aryl halide compound 3 and the organoboronic acid compound 4 as described herein. Path B uses the aryl halide 1-iodo-3-formyl benzene 6 and the organoboronic acid 1-(phenylsulfonyl)-1H-indol-2-yl-boronic acid 7. Compound structures are shown in FIG. 1A.

A mixture of 2-bromo-1-(phenylsulfonyl)-1H-indole 3 (330 mg, 0.99 mmol), tetrakis(triphenylphosphine)palladium(0) (34 mg, 0.3 μmol) and 3-formylphenyl boric acid 4 (177 mg, 1.18 mmol) in dimethoxyethane (DME) (10 mL) with sodium carbonate (1 mL of 2 M in deoxygenated water) was stirred and heated to reflux for 2 h until bromoinodole 3 was not detected on TLC. The mixture was cooled to room temperature (rt) and poured into EtOAc (20 mL) and extracted with EtOAc. The combined organic layers were washed with saturated NH₄Cl and water and dried over MgSO₄. The solvent was removed in vacuo and then purified by flash column chromatography on silica gel using EtOAc/Hx (1:5) as an eluent to give compound 5 (336 mg, 94%) as a yellowish solid. Mp 126-138° C.; Anal. calc. for C₂₁H₁₅NO₃S; C, H, N. ¹H NMR (CDCl₃) δ 10.1 (bs, 1H, CHO), 8.33 (d, J=8.1 Hz, 1H, ArH), 7.98 (s, 2H, ArH), 7.85 (d, J=7.2 Hz, 1H, ArH), 7.63 (t, 1H, J=7.8 Hz, ArH), 7.51-7.29 (m, 8H, ArH) 6.66 (s, 1H, ArH), ¹³C NMR (CDCl₃) δ 192.1, 140.5, 138.6, 137.6, 136.6, 136.1, 134.0, 133.7, 131.1, 130.6, 130.0, 129.0 (2C), 128.5, 126.8 (2C), 125.6, 1254.9, 121.2, 116.8, 114.9.

### Synthesis of (1-benzenesulfonyl-1H-indol-2-yl)-[3-(1-benzenesulfonyl-1H-indol-2-yl)phenyl]methanol (8)

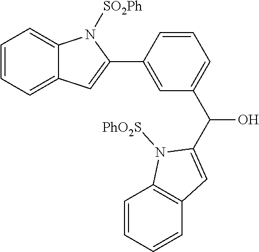

To a solution of protected indole 1 (2.37 g, 6.56 mmol) in 30 mL tetrahydrofuran (THF), 2.0 M LDA solution (4.75 mL, 9.5 mmol) in THF was added within 10 min at −78° C. The solution was stirred at 0° C. for 30 min and subsequently cooled to −78° C. At this temperature, aldehydroindole 5 (2.03 g, 7.88 mmol) dissolved in dry THF (10 mL) was added. The resulting mixture was stirred overnight and allowed to warm to rt. The solution was poured into 100 mL of EtOAc. The combined organic layers were washed with saturated NH₄Cl and water and dried over MgSO₄. The solvent was removed in vacuo and then purified by flash column chromatography on silica gel using EtOAc/Hx (1:3) as an eluent to give compound 8 (3.32 g, 82%) as a yellowish solid. Calculated Mass 618.13, [M+Na⁺] 641.2; Mp 81-83° C.; Anal. calc. for C₃₅H₂₆N₂O₅S₂; C, H, N; ¹H NMR (CDCl₃) δ 8.29 (d, J=8.4 Hz, 1H, ArH), 8.09 (d, J=8.4 Hz, 1H, ArH), 7.73 (d, J=7.5 Hz, 2H, ArH), 7.61 (s, 1H, ArH), 7.56-7.06 (m, 17H, ArH), 6.57 (s, 1H, ArH), 6.48 (s, 1H, ArH), 6.42 (s, 1H, CH), 3.64 (bs, 1H, OH); ¹³C NMR (CDCl₃) δ 143.3, 141.3, 140.0, 138.0, 137.8, 136.9, 136.8, 133.5, 133.1, 132.1, 130.1, 129.6, 128.9, 128.6 (2C), 128.5, 128.1 (2C), 127.1, 126.9, 126.2 (2C), 125.9 (2C), 124.7, 124.5, 124.1, 123.5, 121.0, 120.4, 116.1, 114.2, 113.7, 112.0, 68.8.

### Synthesis of (1-Benzenesulfonyl-1H-indol-2-yl)-[3-(1-benzenesulfonyl-1H-indol-2-yl)phenyl]methane (9)

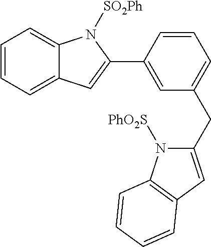

After stirring a solution of compound 8 (201 mg, 0.32 mmol) and triethylsilane (0.1 mL, 0.65 mmol) in 5 mL dry CH₂Cl₂ for 30 min, TFA (0.16 mL, 1.95 mmol) was added. The solution was stirred for 1 h at rt, 10 mL H₂O was added to the solution and the solution was carefully neutralized with solid Na₂CO₃ with ice cooling. The organic phase was separated, dried over Na₂SO₄, and concentrated and then purified by flash column chromatography on silica gel using EtOAc/Hx (1:5) as an eluent to give compound 9 (130 mg, 67%) as a yellowish solid. Calculated Mass 602.13, [M+Na⁺] 625.2; Mp 76-78° C.; Anal. calc. for C₃₅H₂₆N₂O₅S₂ 0.2 C₄H₈O₂; C, H, N; ¹H NMR (CDCl₃) δ 8.30 (d, J=8.1 Hz, 1H, ArH), 8.16 (d, J=8.1 Hz, 1H, ArH), 7.67 (d, J=7.8 Hz, 2H, ArH), 7.51-7.12 (m, 18H, ArH), 6.52 (s, 1H, ArH), 6.30 (s, 1H, CH), 4.34 (s, 2H, CH₂); ¹³C NMR (CDCl₃) δ 141.4, 140.1, 138.5, 137.8, 136.9, 136.7, 134.5, 133.2, 133.0, 132.2, 130.6, 130.2, 129.1, 129.0, 128.7 (2C), 128.5 (2C), 128.0, 127.1, 126.2(2C), 125.9 (2C), 124.4, 123.9, 123.7, 123.1, 120.2, 120.0, 116.1, 114.2, 113.4, 110.9, 34.6.

General Procedure for Preparation of Compounds 10, 11 and 13.

To a solution of compound protected indole (0.56 mmol) in 10 mL ethanol was added a 10% solution of NaOH (227 mg, 5.68 mmol) and the mixture was refluxed for 20 h. Then, ethanol was evaporated, brine and CH₂Cl₂ were added, the organic phase was extracted with CH₂Cl₂ and then was purified by flash column chromatography on silica gel using EtOAc/Hx (1:1) or CH₂Cl₂/Hx (1:1) as an eluent to give the intended free indole compound (69%˜91%).

### Synthesis of (1H-Indol-2-yl)-[3-(1H-indol-2-yl)phenyl]methane (10)

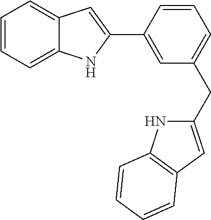

Compound 10 was synthesized from compound 9 by the general procedure described above. Brown solid; Yield 91%; Calculated Mass 322.15, [M−H] 321.2; Mp 193-194° C.; Anal. calc. for C₃₅H₂₆N₂O₅S₂; C, H, N; ¹H NMR (CDCl₃) d 8.30 (bs, 1H, NH), 7.82 (bs, 1H, NH), 7.63-7.54 (m, 4H, ArH), 7.42-7.37 (m, 2H, ArH), 7.27-7.01 (m, 6H, ArH), 8.82 (s, 1H, ArH), 6.34 (s, 1H, ArH), 4.19 (s, 2H, CH₂); ¹³C NMR (CDCl₃) δ 138.8, 136.9, 136.8, 136.3, 135.8, 132.4, 128.9, 128.7, 128.1, 127.7, 124.8, 123.2, 121.9, 120.9, 120.1, 119.9, 119.5, 119.3, 110.4, 110.0, 100.9, 99.7, 34.3.

### Synthesis of (1H-indol-2-yl)-[3-(1H-indol-2-yl)phenyl]methanol (11)

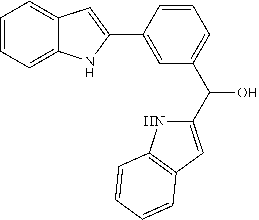

Compound 11 was synthesized from compound 8 by the general procedure described above. Yield 69%; Brown solid; Calculated Mass 338.40, [M−H] 337.2; Anal. calc. for C₂₃H₁₈N₂O; C, H, N; Mp 82-85° C.; ¹H NMR (CDCl₃) δ 8.37 (bs, 1H, NH), 8.26 (bs, 1H, NH), 7.71 (s, 1H, ArH), 7.60-7.06 (m, 11H, ArH), 6.80 (s, 1H, CH), 6.32 (s, 1H, ArH), 5.97 (s, 1H, ArH). ¹³C NMR (CDCl₃) d 141.8, 139.3, 136.9, 136.4, 132.3, 128.8, 128.6, 128.1, 127.5, 125.4, 124.5, 122.3, 122.0, 121.8, 120.2 (2C), 119.8, 119.5, 110.6, 110.5, 100.7, 99.8, 70.2.

### Synthesis of (1-Benzenesulfonyl-1H-indol-2-yl)-[3-(1-benzenesulfonyl-1H-indol-2-yl)phenyl]methanone (12)

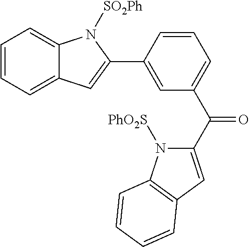

To a solution of compound 8 (325 mg, 0.53 mmol) in dry DMF (10 mL) pyridinium dichromate (PDC, 1.28 mg, 3.4 mmol) was added at 0° C. The mixture was stirred for 20 h at rt. H₂O and CH₂Cl₂ were added, the layers were separated, and the aqueous phase was extracted with CH₂Cl₂. The combined organic extracts were washed with water and dried over MgSO₄. The solvent was evaporated and then purified by flash column chromatography on silica gel using EtOAc/Hx (1:3) as an eluent to give compound 12 (225 mg, 70%) as a yellowish solid. Calculated mass 616.11, [M+Na⁺] 639.2; Mp 189-190° C.; Anal. calc. for C₃₅H₂₄N₂O₅S₂ 0.2 C₄H₈O₂; C, H, N; ¹H NMR (CDCl₃) δ 8.33 (d, J=8.4 Hz, 1H, ArH), 8.20-8.06 (m, 4H, ArH), 7.85 (d, J=8.4 Hz, 1H, ArH), 7.84-7.27 (m, 16H, ArH), 7.13 (s, 1H, ArH), 6.66 (s, 1H, ArH); ¹³C NMR (CDCl₃) δ 186.3, 140.1, 137.9, 137.8, 137.4, 137.2, 136.8, 136.4, 135.1, 133.4, 133.2, 132.2, 130.9, 129.9, 129.6, 128.5 (2C), 128.3 (2C), 128.1, 127.3, 127.0 (2C), 126.7, 126.1 (2C), 124.7, 124.0, 123.8, 122.2, 120.4, 116.8, 116.1, 114.6, 114.0.

### Synthesis of (1H-indol-2-yl)-[3-(1H-indol-2-yl)phenyl]methanone (13)

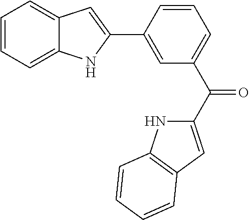

Compound 13 was synthesized from compound 12 by the general procedure described above. Yield 83%; Brown solid; Calculated Mass 336.39, [M−H] 335.3; Mp 206-207° C.; Anal. calc. for C₂₃H₁₆N₂O.0.2 C₄H₈O₂; C, H, N; ¹H NMR (DMSO) δ 8.38 (bs, 1H, NH), 8.18 (bs, 1H, NH), 7.86-7.04 (m, 13H, ArH), 5.77 (s, 1H, ArH). ¹³C NMR (DMSO) δ 186.0, 138.8, 138.1, 137.3, 136.6, 134.2, 129.1, 128.6, 128.4, 127.7, 127.0, 125.8, 124.8, 123.0, 121.9, 120.3, 120.2, 119.5, 112.7, 112.4, 111.4, 99.6.

General Procedure A for Preparation of Compounds 60 and 5 (FIGS. 1i and 1J).

A mixture of arylbromide 1 or compound 3 (0.99 mmol), tetrakis(triphenylphosphine) palladium (0) (34 mg, 0.3 μmol), and 3-formylphenyl boric acid 4 (177 mg, 1.18 mmol) in DME (10 mL) with sodium carbonate (1 mL of 2 M in deoxygenated water) was stirred and heated to reflux for 2 h until arylbromide 1 or compound 3 was not detectable on TLC. The mixture was cooled to rt and poured into EtOAc (20 mL), extracted with EtOAc. The combined organic layers were washed with sat. NH₄Cl, water and dried over anhydrous MgSO₄. The solvent was removed under reduced pressure and then purified by flash column chromatography on silica gel using EtOAc/hexane (1/5, v/v) as an eluent to give target aldehyde compounds.

General Procedure B for Preparation of Compounds 61 and 66 (FIGS. 1i and 1J).

To a solution of bromide 59 (1.38 mmol) in dry THF (10 mL) cooled to −78° C. was added n-BuLi (0.61 mL, 2.5 M, 1.1 eqiv) under argon atmosphere. The solution was stirred for 30 min, aldehyde 60 (1.38 mmol) in anhydrous THF was added, and the solution stirred for 16 h. Water was added to quench the reaction. The reaction solution was extracted with EtOAc, dried with anhydrous MgSO₄. The solvent was removed under reduced pressure and then purified by flash column chromatography on silica gel using EtOAc/hexane (1/1, v/v) as an eluent to give target compounds.

General Procedure D for Preparation of Compounds 62, 64 and 67 (FIGS. 1i and 1J).

To the solution of compound 61, 63, 66 (0.53 mmol) in dry DMF (10 mL) was added pyridinium dichromate (PDC, 1.28 mg, 3.4 mmol) at 0° C. The mixture was stirred for 20 h at rt. Then, H₂O and CH₂Cl₂ were added, the layers were separated, and the aqueous phase was extracted with CH₂Cl₂. The combined organic extracts were washed with water and dried over anhydrous MgSO₄, and the solvent was evaporated, and then purified by flash column chromatography on silica gel using EtOAc/hexane (1/3, v/v) as an eluent to give target compounds.

General Procedure C for Preparation of Compound 63 (FIG. 1i).

To a solution of protected indole 1 (6.56 mmol) in 30 mL THF was added 2.0 M LDA solution (4.75 mL, 9.5 mmol) in THF within 10 min at −78° C., stirring at 0° C. for 30 min and subsequently cooled to −78° C. At this temperature, aryl aldehyde 60 (7.88 mmol), dissolved in dry THF (10 mL), was added. The resulting mixture was stirred overnight and allowed to warm to rt. The solution poured into 100 mL EtOAc. The combined organic layers were washed with sat. NH₄Cl, water and dried over anhydrous MgSO₄. The solvent was removed under reduced pressure and then purified by flash column chromatography on silica gel using EtOAc/hexane (1/3, v/v) as an eluent to give compound 63.

General Procedure E for Preparation of Compounds 65 and 68 (FIGS. 1i and 1J).

To a solution of compound protected indole 64 and 67 (0.56 mmol) in 10 mL ethanol was added a 10% solution of NaOH (227 mg, 5.68 mmol) and the mixture was refluxed for 20 h. Then, ethanol was evaporated, brine and CH₂Cl₂ were added, and the organic phase extracted with CH₂Cl₂ and then purified by flash column chromatography on silica gel using EtOAc/hexane (1/1, v/v) or CH₂Cl₂/hexane (1/1, v/v) as an eluent to give the intended free indole compounds.

### Synthesis of 3′,4′,5′-Trimethoxy-biphenyl-3-carbaldehyde (60)

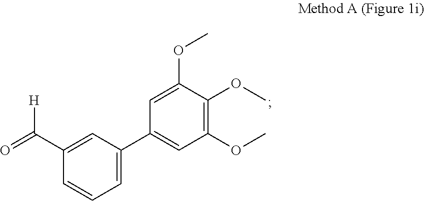

Yield 91%;

MS (ESI) m/z 295.0 ([M+Na]⁺);

¹H NMR (CDCl₃) δ 10.10 (bs, 1H, CHO), 8.07 (t, J=1.7 Hz, 1H, ArH), 7.84 (m, 2H, ArH), 7.85 (t, J=7.8 Hz, 1H, ArH), 6.81 (s, 2H, ArH), 3.95 (s, 6H, OCH₃), 3.91 (s, 3H, OCH₃).

### Synthesis of (3′,4′,5′-Trimethoxy-biphenyl-3-yl)-(3,4,5-trimethoxy-phenyl)-methanol (61)

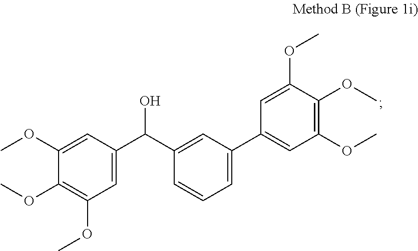

Yield 71%;

MS (ESI) m/z 463.1 ([M+Na]⁺);

¹H NMR (CDCl₃) δ 7.60 (s, 1H, ArH), 7.47-7.31 (m, 3H, ArH), 6.76 (s, 2H, ArH), 6.64 (s, 2H, ArH), 5.81 (s, 1H, CH—OH), 4.00 (s, 6H, OCH₃), 3.90 (s, 3H, OCH₃), 3.81 (s, 9H, OCH₃), 2.97 (s, 1H, OH).

### Synthesis of (3′,4′,5′-Trimethoxy-biphenyl-3-yl)-(3,4,5-trimethoxy-phenyl)-methanone (62)

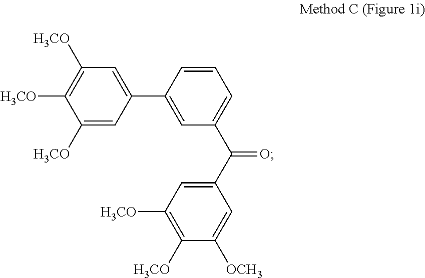

Yield 85%;

MS (ESI) m/z 461.1 ([M+Na]⁺);

¹H NMR (300 MHz, CDCl₃) δ 8.00 (t, J=1.5 Hz, 1H, ArH), 7.79 (m, 1H, ArH), 7.72 (dd, J=7.5, 1.5 Hz, 1H, ArH), 7.55 (t, J=7.5 Hz, 1H, ArH), 7.12 (s, 2H, ArH), 6.81 (s, 2H, ArH), 3.96 (s, 3H, OCH₃), 3.94 (s, 6H, OCH₃), 3.90 (s, 3H, OCH₃), 3.89 (s, 6H, OCH₃).

### Synthesis of (1-Benzenesulfonyl-1H-indol-2-yl)-(3′,4′,5′-trimethoxy-biphenyl-3-yl)-methanol (63)

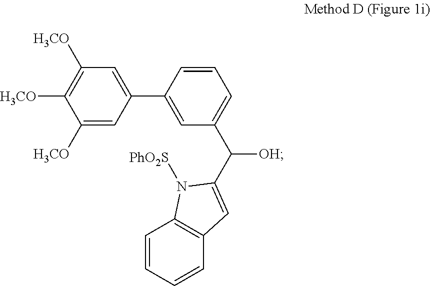

Yield 84%;

MS (ESI) m/z 552.2 ([M+Na]⁺);

¹H NMR (300 MHz, CDCl₃) δ 8.00 (t, J=1.5 Hz, 1H, ArH), 7.79 (m, 1H, ArH), 7.72 (dd, J=7.5, 1.5 Hz, 1H, ArH), 7.55 (t, J=7.5 Hz, 1H, ArH), 7.12 (s, 2H, ArH), 6.81 (s, 2H, ArH), 3.96 (s, 3H, OCH₃), 3.94 (s, 6H, OCH₃), 3.90 (s, 3H, OCH₃), 3.89 (s, 6H, OCH₃).

### Synthesis of (1-Benzenesulfonyl-1H-indol-2-yl)-(3′,4′,5′-trimethoxy-biphenyl-3-yl)-methanone (64)

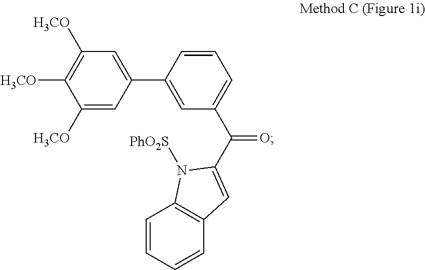

Yield 85%;

MS (ESI) m/z 528.3 ([M+H]⁺);

¹H NMR (300 MHz, CDCl₃) δ 8.21 (s, 1H, ArH), 8.17-8.06 (m, 3H, ArH), 7.94 (d, J=7.8 Hz, 1H, ArH), 7.82 (d, J=7.8 Hz, 1H, ArH), 7.58-7.46 (m, 6H, ArH), 7.34-7.32 (d, J=7.8 Hz, 1H, ArH), 7.01 (s, 1H, ArH), 6.83 (s, 2H, ArH), 3.95 (s, 6H, OCH₃), 3.91 (s, 3H, OCH₃).

### Synthesis of (1H-Indol-2-yl)-(3′,4′,5′-trimethoxy-biphenyl-3-yl)-methanone (65)

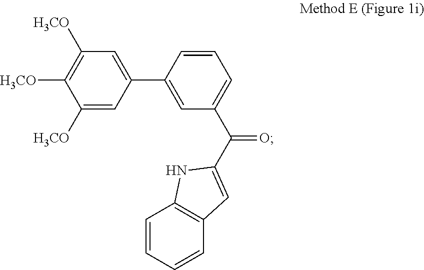

Yield 75%;

MS (ESI) m/z 385.9 ([M−H]⁻);

¹H NMR (300 MHz, CDCl₃) δ 9.62 (bs, 1H, NH), 8.17 (s, 1H, ArH), 7.98 (d, J=7.8 Hz, 1H, ArH), 7.82 (d, J=7.8 Hz, 1H, ArH), 7.73 (d, J=7.8 Hz, 1H, ArH), 7.61 (t, J=7.8 Hz, 1H, ArH), 7.52 (d, J=8.4 Hz, 1H, ArH), 7.40 (t, J=7.8 Hz, 1H, ArH), 7.21-7.16 (m, 2H, ArH), 6.86 (s, 2H, ArH), 3.95 (s, 6H, OCH₃), 3.93 (s, 3H, OCH₃).

### Synthesis of [3-(1-Benzenesulfonyl-1H-indol-2-yl)-phenyl]-(3,4,5-trimethoxy-phenyl)-methanol (66)

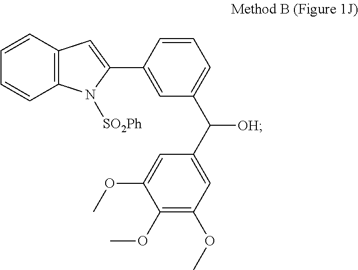

Yield 71%;

MS (ESI) m/z 552.2 ([M+H]⁺);

¹H NMR (300 MHz, CDCl₃) δ 8.30 (d, J=8.1 Hz, 1H, ArH), 7.63 (s, 1H, ArH), 7.49-7.16 (m, 12H, ArH), 6.71 (s, 2H, ArH), 6.56 (s, 1H, CH—OH), 5.87 (bs, 1H, CH—OH), 3.87 (s, 6H, OCH₃), 3.85 (s, 3H, OCH₃).

### Synthesis of [3-(1-Benzenesulfonyl-1H-indol-2-yl)-phenyl]-(3,4,5-trimethoxy-phenyl)-methanone (67)

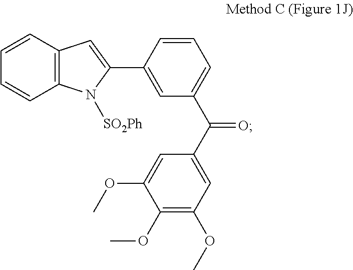

Yield 69%;

MS (ESI) m/z 550 ([M+Na]⁺);

¹H NMR (300 MHz, CDCl₃) δ 8.31 (d, J=8.4 Hz, 1H, ArH), 8.02 (m, 2H, ArH), 7.73 (m, 1H, ArH), 7.64 (t, J=7.5 Hz, 1H, ArH), 7.50-7.31 (m, 8H, ArH), 7.27 (s, 2H, ArH), 6.65 (s, 1H, ArH), 4.01 (s, 3H, OCH₃), 3.99 (s, 6H, OCH₃).

### Synthesis of [3-(1H-Indol-2-yl)-phenyl]-(3,4,5-trimethoxy-phenyl)-methanone (68)

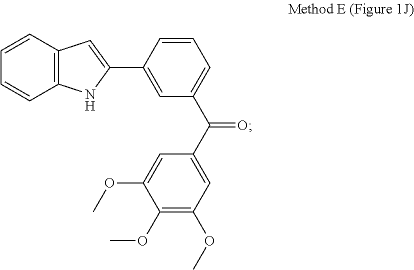

Yield 95%;

MS (ESI) m/z 385.9 ([M−H]⁻);

¹H NMR (300 MHz, CDCl₃) δ 8.94 (bs, 1H, NH), 8.17 (s, 1H, ArH), 7.94 (d, J=7.8 Hz, 1H, ArH), 7.69 (m, 2H, ArH), 7.55 (t, J=7.6 Hz, 1H, ArH), 7.41 (d, J=8.1 Hz, 1H, ArH), 7.25-7.15 (m, 2H, ArH), 7.12 (s, 2H, ArH), 6.92 (s, 1H, ArH), 3.98 (s, 3H, OCH₃), 3.85 (s, 6H, OCH₃).

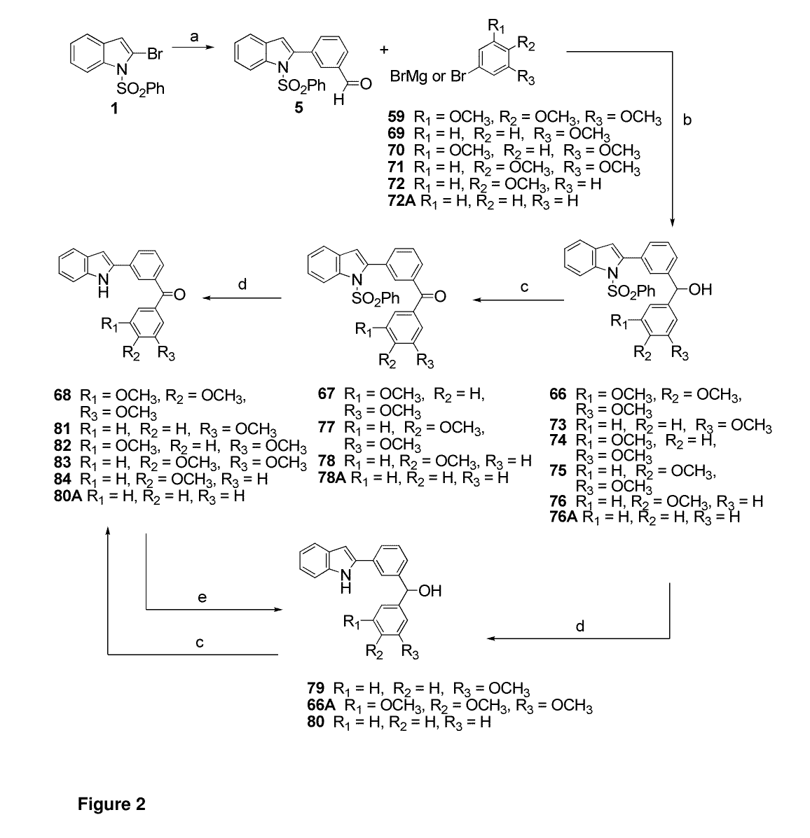

General Procedure for Preparation of Compounds 68, 79-80 and 81-84 (FIG. 2).

The indole aldehyde 5 was treated with corresponding phenylmagnesium bromides 59, 69-72, 72A to form their respective alcohols 66, 73-76, 76A. The alcohols were oxidized with pyridinium dichromate or Dess-Martin periodinate to form protected ketones 67 and 77-78, 78A. Deprotection using aqueous NaOH in ethanol afforded targets 68, 80A, and 81-84. Alcohol 66A was synthesized by catalytic hydrogenation of 68. Compound 80A was prepared by oxidation of compound 80.

### [3-(1-Benzenesulfonyl-indol-2-yl)-phenyl]-(3-methoxy-phenyl)-methanol (73)

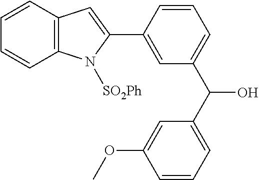

Yield 40%. The crude material was used for the next step. MS (ESI): calculated for C₂₈H₂₃NO₄S 469.1, found 492.1 [M+Na]⁺.

### [3-(1-Benzenesulfonyl-indol-2-yl)-phenyl]-(3,5-dimethoxy-phenyl)-methanol (74)

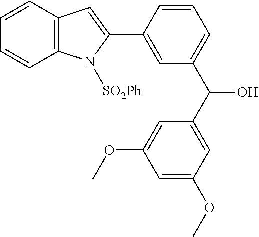

Crude yield 100%. The crude material was used for the next step. ¹H NMR (300 MHz, CDCl₃) δ 3.79 (s, 6H), 4.74-4.83 (m, 1H), 5.81-5.88 (m, 1H), 6.61-6.68 (m, 1H), 7.14-7.23 (m, 2H), 7.29-7.36 (m, 3H), 7.36-7.52 (m, 9H), 7.48-7.59 (m, 1H), 8.25-8.40 (m, 1H).

### [3-(1-Benzenesulfonyl-indol-2-yl)-phenyl]-(3,4-dimethoxy-phenyl)-methanol (75)

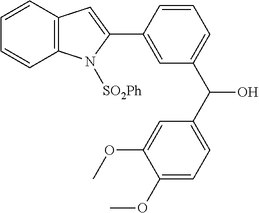

Yield 45%. MS (ESI): calculated for C₂₉H₂₅NO₅S, 499.2, found 522.2 [M+Na]⁺. ¹H NMR (500 MHz, CDCl₃) δ 2.28 (d, J=3.42 Hz, 1H), 3.87 (m, 6H), 5.88 (dd, J=2.69, 0.73 Hz, 1H), 6.54 (s, 1H), 6.86 (d, J=8.06 Hz, 1H), 6.96-7.02 (m, 2H), 7.13-7.22 (m, 2H), 7.22-7.30 (m, 1H), 7.31-7.45 (m, 6H), 7.58 (s, 1H), 8.30 (d, J=8.54 Hz, 1H).

### [3-(1-Benzenesulfonyl-indol-2-yl)-phenyl]-(4-methoxy-phenyl)-methanol (76)

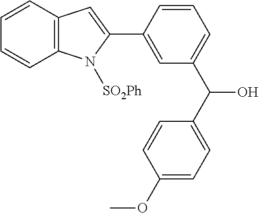

Crude yield 100%. The crude material was used for the next step. MS (ESI): calculated for C₂₈H₂₃NO₄S 469.1, found 492.1 [M+Na]⁺.

### [3-(1-Benzenesulfonyl-1H-indol-2-yl)-phenyl]-phenyl-methanol (76A)

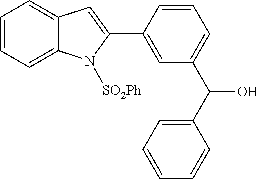

Yield 73% clear resin. MS (ESI): calculated for C₂₇H₂₁NO₃S, 439.1, found 435.9 [M−H]⁻, 438.1 [M+H]⁺.

### [3-(1-Benzenesulfonyl-1H-indol-2-yl)-phenyl]-phenyl-methanone (78A)

Yield 61% white resin.

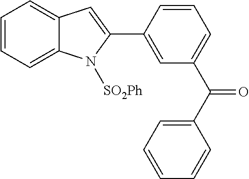

### [3-(1H-Indol-2-yl)-phenyl]-phenyl-methanone (80A)

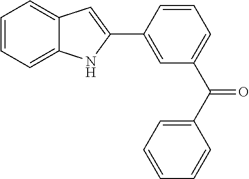

MS (ESI): calculated for C₂₁H₁₅NO, 297.12, found 295.9 [M−H]⁻, 298.0 [M+H]⁺. ¹H NMR (500 MHz, DMSO-d₆) δ 6.97-7.05 (m, 2H), 7.07-7.17 (m, 1H), 7.37-7.45 (m, 1 H), 7.53-7.58 (m, 1H), 7.58-7.68 (m, 4H), 7.69-7.77 (m, 1H), 7.79-7.86 (m, 2H), 8.14-8.20 (m, 1H), 8.20-8.26 (m, 1H), 11.67-11.77 (m, 1H).

### [3-(1-Benzenesulfonyl-indol-2-yl)-phenyl]-(3,5-dimethoxy-phenyl)-methanone (67)

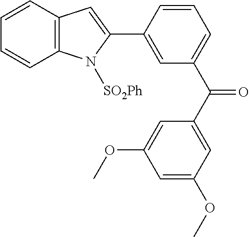

MS (ESI): calculated for C₂₉H₂₃NO₅S, 497.1, found 520.2 [M+Na]⁺. ¹H NMR (300 MHz, CDCl₃) δ 3.89 (s, 6H), 6.57-6.79 (m, 2H), 7.01-7.10 (m, 2H), 7.23-7.51 (m, 5H), 7.54-7.69 (m, 2H), 7.70-7.89 (m, 1H), 7.90-8.02 (m, 2H), 8.26-8.39 (m, 1H), 10.06-10.15 (m, 1H).

### [3-(1-Benzenesulfonyl-indol-2-yl)-phenyl]-(3,4-dimethoxy-phenyl)-methanone (77)

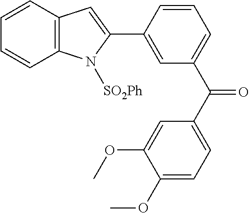

Yield 96%. The crude material was used for the next step. MS (ESI): calculated for C₂₉H₂₃NO₅S, 497.13, found 520.2 [M+Na]⁺.

### [3-(1-Benzenesulfonyl-indol-2-yl)-phenyl]-(4-methoxy-phenyl)-methanone (78)

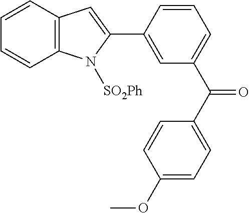

Crude yield 94%. The crude material was used for the next step. MS (ESI): calculated for C₂₈H₂₁NO₄S, 467.1, found 492.1 [M+Na]⁺.

### [3-(1H-Indol-2-yl)-phenyl]-(3-methoxy-phenyl)-methanol (79)

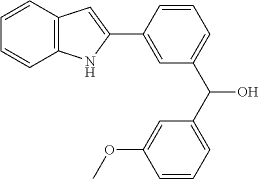

Crude yield 100%. The crude material was used for the next step.

### [3-(1H-Indol-2-yl)-phenyl]-(3,4,5-trimethoxy-phenyl)-methanol (66A)

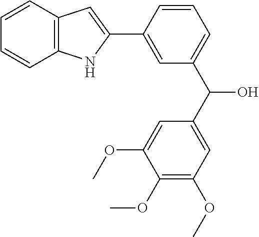

MS (ESI): calculated for C₂₄H₂₃NO₄, 389.16, found 388.0 [M−H]⁻, 412.2 [M+Na]⁺. ¹H NMR (500 MHz, DMSO-d₆) δ 3.61 (s, 3H), 3.76 (s, 6H), 5.65-5.74 (m, 1H), 5.98 (br. s, 1H), 6.75-6.81 (m, 2H), 6.88 (br. s, 1H), 6.95-7.03 (m, 1H), 7.05-7.13 (m, 1H), 7.30-7.35 (m, 1H), 7.35-7.44 (m, 2H), 7.48-7.59 (m, 1H), 7.64-7.76 (m, 1H), 7.96 (br. s, 1H), 11.55 (br. s, 1H).

### [3-(1H-Indol-2-yl)-phenyl]-(3-methoxy-phenyl)-methanone (81)

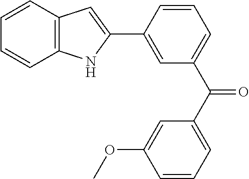

Yield 23%. ¹H NMR (300 MHz, CDCl₃) δ 3.90 (s, 3H), 6.84-6.99 (m, 1H), 7.10-7.27 (m, 3H), 7.37-7.49 (m, 4H), 7.52-7.62 (m, 1H), 7.62-7.70 (m, 1H), 7.70-7.77 (m, 1H), 7.87-7.99 (m, 1H), 8.07-8.17 (m, 1H), 8.38-8.56 (m, 1H).

### (3,5-Dimethoxy-phenyl)-[3-(1H-indol-2-yl)-phenyl]-methanone (82)

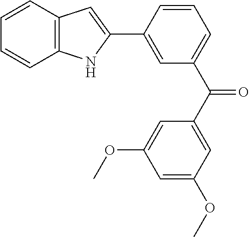

MS (ESI): calculated for C₂₃H₁₉NO₃, 357.14, found 355.9 [M−H]⁻. ¹H NMR (300 MHz, CDCl₃) δ 3.85 (m, 6H), 6.72 (m, 1H), 6.91 (m, 1H), 6.98 (m, 2H), 7.10-7.27 (m, 2 H), 7.37-7.49 (m, 1H), 7.52-7.62 (m, 1H), 7.62-7.70 (m, 1H), 7.70-7.77 (m, 1H), 7.87-7.94 (m, 1H), 8.08-8.15 (m, 1H), 8.42-8.55 (br. s, 1H).

### (3,4-Dimethoxy-phenyl)-[3-(1H-indol-2-yl)-phenyl]-methanone (83)

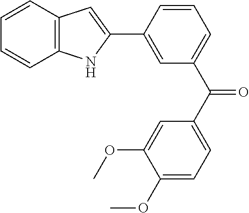

Yield 79%. MS (ESI): calculated for C₂₃H₁₉NO₃, 357.1, found 356.0 [M−H]⁻. ¹H NMR (300 MHz, CDCl₃) δ 3.98 (m, 6H), 6.85-7.00 (m, 2H), 7.10-7.19 (m, 1H), 7.19-7.26 (m, 1H), 7.43 (d, J=8.54 Hz, 2H), 7.53-7.60 (m, 2H), 7.62-7.72 (m, 2H), 7.86-7.93 (m, 1H), 8.06 (s, 1H), 8.46 (br. s, 1H).

### [3-(1H-Indol-2-yl)-phenyl]-(4-methoxy-phenyl)-methanone (84)

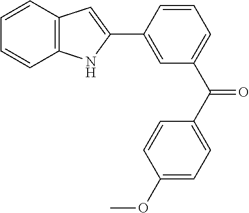

Yield 85%. MS (ESI): calculated for C₂₂H₁₇NO₂, 327.1, found 326.9 [M−H]⁻. ¹H NMR (300 MHz, CDCl₃) δ 3.93 (s, 3H), 6.88-6.94 (m, 1H), 7.01 (d, J=8.85 Hz, 2H), 7.11-7.19 (m, 1H), 7.19-7.27 (m, 1H), 7.37-7.47 (m, 1H), 7.51-7.60 (m, 1H), 7.67 (s, 2H), 7.90 (d, J=8.85 Hz, 3H), 8.06 (s, 1H), 8.44-8.63 (m, 1H),

### (4-Fluoro-phenyl)-[3-(1H-indol-5-yl)-phenyl]-methanone (125)

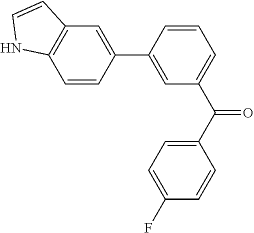

MS (ESI): calculated for C₂₁H₁₄FNO, 315.11, found [M−H]⁻, [M+H]⁺. ¹H NMR (300 MHz, DMSO-d₆) δ 6.46-6.54 (m, 1H), 6.75-6.84 (m, 2H), 7.35-7.65 (m, 5H), 7.67-7.77 (m, 2H), 7.82-7.95 (m, 3H), 11.13-11.24 (m, 1H).

### [3-(1-Benzenesulfonyl-1H-indol-2-yl)-phenyl]-(4-fluoro-phenyl)-methanone (126)

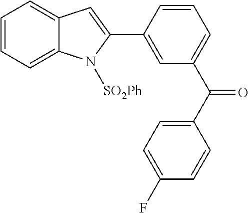

MS (ESI): calculated for C₂₇H₁₈FNO₃S, 455.10, found [M−H]⁻, [M+H]⁺. ¹H NMR (300 MHz, CDCl₃) δ 6.64 (s, 1H), 7.17-7.50 (m, 10H), 7.59 (s, 1H), 7.74 (s, 1H), 7.92 (s, 2H), 8.00 (dd, J=8.54, 5.49 Hz, 2H), 8.32 (d, J=8.24 Hz, 1H).

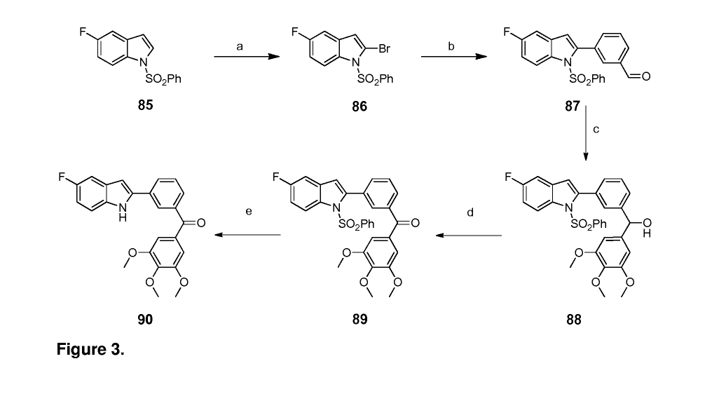

### General Synthesis of (3-(5-fluoro-1H-indol-2-yl)phenyl)(3,4,5-trimethoxyphenyl)methanone (90) (FIG. 3)

Compound 90 was prepared in much the same way as 68 and 81-84. A solution of 1-(phenylsulfonyl)-5-fluoroindole was treated with butyllithium and then cyanogen bromide. The resulting bromide, 86, was treated with 3-formylphenyl boronic acid under Suzuki coupling conditions to produce aldehyde 87. Treatment of 87 with 3,4,5-trimethoxyphenyl lithium gave 88, which was oxidized with PDC to produce 89. Hydrolysis of 89 in basic conditions produced 90.

### Synthesis of 1-Benzenesulfonyl-2-bromo-5-fluoro-1H-indole (86)

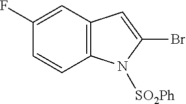

A THF solution of 1-(phenylsulfonyl)-5-fluoroindole was treated with 2-M LDA (1.1 equiv.) at −78° C. and stirred for 2 h before BrCN (1.25 equiv.) was added as a solid. The mixture was stirred and allowed to come to rt overnight. The reaction was quenched with 3% NaHCO₃, concentrated by rotary evaporation, and extracted with dichloromethane. The organic extract was washed with water, dried with MgSO₄, and concentrated to a solid. The crude product was used in the next step without thorough purification or characterization.

### Synthesis of 3-(1-Benzenesulfonyl-5-fluoro-1H-indol-2-yl)-benzaldehyde (87)

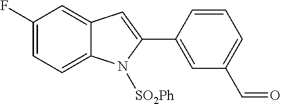

A mixture of 86 (2.1 g), 3-benzaldehyde boronic acid (1.0 g, 1.2 equiv.), Pd(PPh₃)₄ (270 mg, 0.040 equiv.), DMF (100 mL), and aqueous K₂CO₃ (2-M, 5.8 mL) was refluxed under argon for 16 h. The mixture was brought to rt, diluted with 100 mL water and extracted with 100 mL EtOAc. The organic layer was separated, washed with 5×50 mL water, and dried (MgSO₄), and concentrated to a paste. The paste was chromatographed on silica with an EtOAc/hexanes mixture to afford 1.15 g 87 (53%).

MS (ESI): calculated for C₂₁H₁₄FNO₃S, 379.07, found [M−H]⁻, [M+H]⁺. The chromatographed product was used in the next step without thorough purification or characterization.

### Synthesis of [3-(1-Benzenesulfonyl-5-fluoro-1H-indol-2-yl)-phenyl]-(3,4,5-trimethoxy-phenyl)-methanol (88)

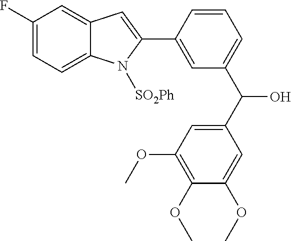

A 25-mL THF solution of 1-bromo-3,4,5-trimethoxybenzene (488 mg, 1.5 equiv.) was cooled to −78° C. under argon before treatment with BuLi (0.99 mL, 2-M, 1.5 equiv). The mixture was stirred for 1 h before 87 (500 mg, 1.00 equiv.) was added as a solid. The mixture was allowed to warm to rt overnight. The reaction was quenched with 3% NaHCO₃, concentrated by rotary evaporation, and extracted with 50 mL EtOAc. The organic layer was separated, washed with 2×50 mL 3% NaHCO₃ and 50 mL water before it was dried (MgSO₄), and concentrated to a paste. The paste was chromatographed on silica with an EtOAc/hexanes mixture to afford 354 mg 88 (49%). MS (ESI): calculated for C₃₀H₂₆FNO₆S, 547.15, found [M−H]⁻, [M+H]⁺. The chromatographed product was used in the next step without thorough purification or characterization.

### Synthesis of [3-(1-Benzenesulfonyl-5-fluoro-1H-indol-2-yl)-phenyl]-(3,4,5-trimethoxy-phenyl)-methanone (89)

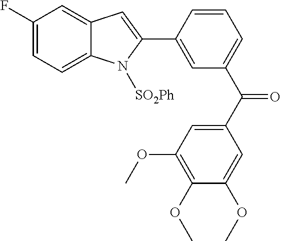

To a 50-mL DMF solution of 88 (300 mg, 1.00 equiv.) was added PDC (1.67 g, 6.5 equiv.). The mixture was stirred at rt overnight. The mixture was diluted with 100 mL water and extracted with 100 mL EtOAc. The organic layer was separated, washed with 5×50 mL water, and dried (MgSO₄), and concentrated to a paste. The paste was chromatographed on silica with an EtOAc/hexanes mixture to afford 176 mg 89 (59%).

MS (ESI): calculated for C₃₀H₂₄FNO₆S, 545.13, found [M−H]⁻, [M+H]⁺.

¹H NMR (500 MHz, CDCl₃) δ 3.92-3.95 (m, 6H), 3.95-3.98 (m, 3H), 6.49-6.61 (m, 1 H), 7.06-7.14 (m, 2H), 7.19-7.24 (m, 2H), 7.28-7.36 (m, 2H), 7.38-7.44 (m, 2H), 7.45-7.52 (m, 1H), 7.56-7.63 (m, 1H), 7.64-7.71 (m, 1H), 7.97 (br. s., 1H), 8.16-8.29 (m, 1H), 8.88 (br s, 1H).

### Synthesis of [3-(5-Fluoro-1H-indol-2-yl)-phenyl]-(3,4,5-trimethoxy-phenyl)-methanone (90)

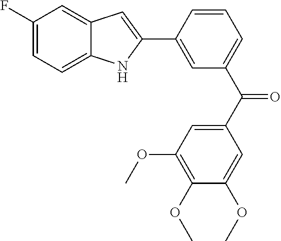

To a 14 mL DMF solution of 89 (176 mg) was added 14 mL water and NaOH (258 mg, 20 equiv). The resulting turbid mixture was refluxed overnight. Concentration and chromatography on silica gel with an EtOAc/hexanes mixture afforded 90. MS (ESI): calculated for C₂₄H₂₀FNO₄, 405.14, found [M−H]⁻, [M+H]⁺.

¹H NMR (500 MHz, CDCl₃) δ 3.88 (s, 6H), 3.97 (s, 3H), 6.86 (br s, 1H), 6.97 (t, J=8.91 Hz, 2H), 7.11 (br s, 2H), 7.28-7.31 (m, 1H), 7.31-7.37 (m, 1H), 7.57 (t, J=7.57 Hz, 2H), 7.71 (d, J=7.32 Hz, 2H), 7.89 (d, J=7.32 Hz, 1H), 8.09 (br s, 1H), 8.50 (br. s, 1H).

### General Procedure for Preparation of Compounds 112-118 and 148, 119-121, 123a, 123b and 127 (FIG. 4)

The synthesis of compounds 112-118, 148 and 119-121 as well as 123(a,b) is described in FIG. 4. Protection of the indole or benzimidazole NH of compounds 91-97 and 145 with a phenylsulfonyl moiety yielded intermediates 98-104 and 146, which were deprotonated by tert-butyllithium followed by coupling with the 3,4,5-trimethoxy benzoyl chloride to generate compounds 105-111 and 147. Removal of the protecting group from 105-111 and 147 gave compounds 112-118 and 148. Compounds 112, 113 were methylated by methyliodide to obtain compounds 119 and 121, respectively. Benzylation of compound 112 provided compound 120.

The synthesis of compounds 123a and 123b was very straightforward with a one-step reaction involved. The commercial starting materials 122a and 122b were treated directly by tert-butyllithium followed by benzoylation to achieve the desired products 123a and 123b. Compound 127 (see Table 3 infra for structure) was separated as a side product from the synthesis of compound 123a.

### General Procedure for the Synthesis of 1-(phenylsulfonyl)-1H-indole (98-103, 146), 1-(phenylsulfonyl)-1H-benzo[d]imidazole (104)

To a solution of indoles (91-96 and 145) or benzimidazole (97) (3.5 g, 10 mmol) in anhydrous THF (200 mL) at 0° C. was added sodium hydride (60% dispersion in mineral oil, 48 mg, 12 mmol, 1.2 equiv) and stirred for 20 min. Benzenesulfonyl chloride (2.1 g, 12 mmol, 1.2 equiv) was added and the reaction mixture was stirred overnight. After dilution by 200 mL of saturated NaHCO₃ solution (aqueous), the reaction mixture was extracted by ethyl acetate (600 mL). The organic layer was dried over magnesium sulfate and concentrated. The residue was purified by flash column chromatography (hexane:ethyl acetate 2:1) to give a pale solid. Yield: 40%-95%.

### 6-Chloro-1-(phenylsulfonyl)-1H-indole (98)

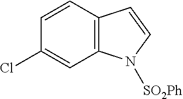

Yield: 82.3%. ¹H NMR (500 MHz, CDCl₃) δ 8.09 (s, 1H), 7.95 (d, J=7.8 Hz, 2 H), 7.59-7.66 (m, 2H), 7.46-7.58 (m, 3H), 7.24-7.30 (m, 1H), 6.70 (d, J=3.7 Hz, 1H). MS (ESI) calcd for C₁₄H₁₀ClNO₂S 291.0, found 314.0 [M+Na]⁺.

### 6-Fluoro-1-(phenylsulfonyl)-1H-indole (99)

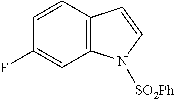

Yield: 92.5%. ¹H NMR (500 MHz, CDCl₃) δ 7.95 (d, J=7.8 Hz, 2H), 7.80 (dd, J=2.0, 9.6 Hz, 1H), 7.58-7.66 (m, 2H), 7.47-7.57 (m, 3H), 7.06 (dt, J=2.44, 8.9 Hz, 1H), 6.70 (d, J=3.6 Hz, 1H). MS (ESI) calcd for C₁₄H₁₀FNO₂S 275.1, found 298.1 [M+Na]⁺.

### 5-Chloro-1-(phenylsulfonyl)-1H-indole (101)

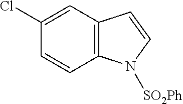

Yield: 96.3%. ¹H NMR (500 MHz, CDCl₃) δ 7.99 (d, J=9.0 Hz, 1H), 7.92 (d, J=7.8 Hz, 2H), 7.68-7.59 (m, 2H), 7.56 (d, J=1.7 Hz, 1H), 7.47-7.55 (m, 2H), 7.31-7.36 (m, 1H), 6.67 (d, J=3.4 Hz, 1H).

MS (ESI) calcd for C₁₄H₁₀ClNO₂S 291.0, found 314.0 [M+Na]⁺.

### 6-Methyl-1-(phenylsulfonyl)-1H-indole (102)

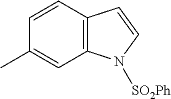

Yield: 95.1%. ¹H NMR (500 MHz, CDCl₃) δ 7.94 (d, J=7.8 Hz, 2H), 7.88 (s, 1H), 7.57-7.63 (m, 1H), 7.56 (d, J=3.7 Hz, 1H), 7.44-7.53 (m, 3H), 7.12 (d, J=7.8 Hz, 1H), 6.68 (d, J=3.7 Hz, 1H), 2.54 (s, 3H). MS (ESI) calcd for C₁₅H₁₃NO₂S 271.1, found 294.0 [M+Na]⁺.

### 5-Fluoro-1-(phenylsulfonyl)-1H-indole (103)

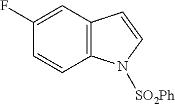

Yield: 88.6%. ¹H NMR (500 MHz, CDCl₃) δ 8.00 (dd, J=4.4, 9.0 Hz, 1H), 7.92 (d, J=7.6 Hz, 2H), 7.67 (d, J=3.7 Hz, 1H), 7.62 (t, J=7.4 Hz, 1H), 7.51 (t, J=7.8 Hz, 2 H), 7.24 (dd, J=2.4, 8.8 Hz, 1H), 7.11 (dt, J=2.4, 9.0 Hz, 1H), 6.69 (d, J=3.7 Hz, 1H). MS (ESI) calcd for C₁₄H₁₀FNO₂S 275.1, found 298.1 [M+Na]⁺.

### 5-Methoxy-1-(phenylsulfonyl)-1H-indole (146)

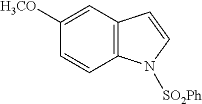

Yield: 92.6%. ¹H NMR (500 MHz, CDCl₃) δ 7.84-7.90 (m, 3H), 7.51-7.53 (m, 2 H), 7.43 (t, J=8.0 Hz, 2H), 6.98 (d, J=2.0 Hz, 1H), 6.93 (dd, J=9.0 Hz, 2.5 Hz, 1H), 6.60 (d, J=3.5 Hz, 1H), 3.82 (s, 3H). MS (ESI) calcd for C₁₅H₁₃NO₃S 287.1, found 310.0 [M+Na]⁺.

### 1-(Phenylsulfonyl)-1H-benzo[d]imidazole (104)

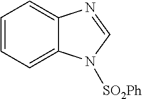

Yield: 99%. ¹H NMR (500 MHz, CDCl₃) δ 8.46 (s, 1H), 8.07 (d, J=7.6 Hz, 2H), 7.94 (d, J=8.3 Hz, 1H), 7.84 (d, J=7.6 Hz, 1H), 7.70 (t, J=7.6 Hz, 1H), 7.59 (t, J=7.8 Hz, 2H), 7.40-7.51 (m, 2H). MS (ESI) calcd for C₁₃H₁₀N₂O₂S 258.1, found 259.0 [M+H]⁺.

### General Procedure for the synthesis of (1-(phenylsulfonyl)-1H-indol-2-yl)(3,4,5-trimethoxyphenyl)methanone (105-110, 147) and (1-(phenylsulfonyl)-1H-benzo[d]imidazol-2-yl)(3,4,5-trimethoxyphenyl)methanone (111), benzo[d]thiazol-2-yl(3,4,5-trimethoxyphenyl)methanone (123a), benzo[d]oxazol-2-yl(3,4,5-trimethoxyphenyl)methanone (123b)

To a solution of 1-(phenylsulfonyl)-1H-indoles (98-103, 146) or 1-(phenylsulfonyl)-1H-benzo[d]imidazole (104) or benzothiazole (122a), benzoxazole (122b) (1.6-2.3 g, 5.0 mmol) in anhydrous THF (30 mL) at −78° C. was added 1.7 M tert-butyllithium in pentane (3.5 mL, 6.0 mmol, 1.2 equiv) and stirred for 10 min. 3,4,5-Trimethoxybenzoyl chloride (1.4 g, 6.0 mmol, 1.2 equiv) was added at −78° C. and stirred overnight. The reaction mixture was diluted with 100 mL of saturated NaHCO₃ solution (aqueous) and extracted by ethyl acetate (300 mL). The organic layer was dried over magnesium sulfate and concentrated. The residue was purified by flash column chromatography (hexane:ethyl acetate 3:1) to give a white solid. Yield: 5%-45%.

### (6-Chloro-1-(phenylsulfonyl)-1H-indol-2-yl)(3,4,5-trimethoxyphenyl)methanone (105)

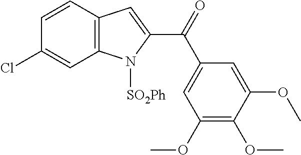

Yield: 34.7%. ¹H NMR (500 MHz, CDCl₃) δ 8.17-8.25 (m, 3H), 7.69-7.75 (m, 1 H), 7.60-7.66 (m, 2H), 7.58 (d, J=8.3 Hz, 1H), 7.37 (dd, J=1.5, 8.3 Hz, 1H), 7.32 (d, J=9.3 Hz, 3H), 6.96 (s, 1H), 4.02 (s, 3H), 3.95 (s, 6H). MS (ESI) calcd for C₂₄H₂₀ClNO₆S 485.1, found 508.0 [M+Na]⁺.

### (6-Fluoro-1-(phenylsulfonyl)-1H-indol-2-yl)(3,4,5-trimethoxyphenyl)methanone (106)

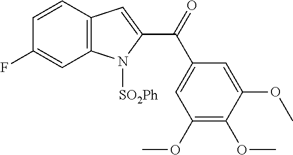

Yield: 34.7%; mp 171-173° C. ¹H NMR (500 MHz, CDCl₃) δ 8.22 (d, J=7.5 Hz, 2 H), 7.93 (dd, J=1.8, 9.8 Hz, 1H), 7.67-7.75 (m, 1H), 7.57-7.67 (m, 3H), 7.29-7.34 (s, 2H), 7.15 (dt, J=2.20, 8.91 Hz, 1H), 6.98 (s, 1H), 4.02 (s, 3H), 3.95 (s, 6H). MS (ESI) calcd for C₂₄H₂₀FNO₆S 469.1, found 492.0 [M+Na]⁺. HPLC1: t_{R} 4.01 min, purity >99%.

### (6-Methoxy-1-(phenylsulfonyl)-1H-indol-2-yl)(3,4,5-trimethoxyphenyl)methanone (107)

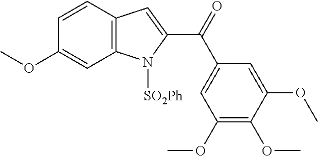

Yield: 26.4%; mp 156-158° C. ¹H NMR (500 MHz, CDCl₃) δ 8.18 (d, J=7.8 Hz, 2 H), 7.73 (s, 1H), 7.71-7.65 (m, 1H), 7.57-7.63 (m, 2H), 7.53 (d, J=8.5 Hz, 1H), 7.32 (s, 2H), 7.01 (dd, J=1.8, 8.7 Hz, 1H), 6.99 (s, 1H), 4.01 (s, J=5.9 Hz, 6H), 3.95 (s, 6 H). MS (ESI) calcd for C₂₅H₂₃NO₇S 481.1, found 504.1 [M+Na]⁺. HPLC1: t_{R} 3.99 min, purity >99%.

### (5-Chloro-1-(phenylsulfonyl)-1H-indol-2-yl)(3,4,5-trimethoxyphenyl)methanone (108)

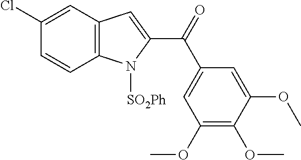

Yield: 39.6%. ¹H NMR (500 MHz, CDCl₃) δ 8.08-8.20 (m, 3H), 7.65-7.72 (m, 1 H), 7.56-7.65 (m, 3H), 7.45-7.53 (m, 1H), 7.28-7.35 (m, 2H), 6.92 (s, 1H), 4.02 (s, 3 H), 3.94 (s, 6H). MS (ESI) calcd for C₂₄H₂₀ClNO₆S 485.1, found 508.1 [M+Na]⁺.

### (6-Methyl-1-(phenylsulfonyl)-1H-indol-2-yl)(3,4,5-trimethoxyphenyl)methanone (109)

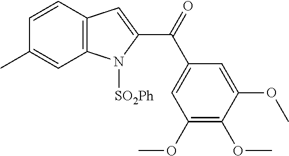

Yield: 26.4%. ¹H NMR (500 MHz, CDCl₃) δ 8.19 (d, J=7.8 Hz, 2H), 8.01 (s, 1 H), 7.65-7.70 (m, 1H), 7.48-7.63 (m, 3H), 7.33 (s, 2H), 7.21 (d, J=8.1 Hz, 1H), 6.98 (s, 1H), 4.02 (s, 3H), 3.92-3.97 (m, 6H), 2.61 (s, 3H). MS (ESI) calcd for C₂₅H₂₃NO₆S 465.1, found 488.0 [M+Na]⁺.

### (5-Fluoro-1-(phenylsulfonyl)-1H-indol-2-yl)(3,4,5-trimethoxyphenyl)methanone (110)

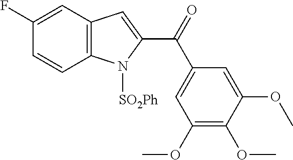

Yield: 41.5%. ¹H NMR (500 MHz, CDCl₃) δ 8.15 (d, J=7.8 Hz, 3H), 7.68 (t, J=7.3 Hz, 1H), 7.59 (t, J=7.7 Hz, 2H), 7.22-7.34 (m, 5H), 6.95 (s, 1H), 3.99-4.05 (m, 3 H), 3.95 (s, 7H). MS (ESI) calcd for C₂₄H₂₀FNO₆S 469.1, found 492.0 [M+Na]⁺.

### (5-Methoxy-1-(phenylsulfonyl)-1H-indol-2-yl)(3,4,5-trimethoxyphenyl)methanone (147)

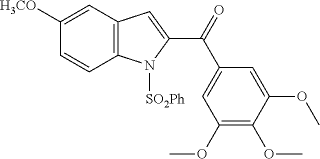

Yield: 32.1%; ¹H NMR (500 MHz, CDCl3) δ 8.03-8.07 (m, 4H), 7.60 (t, J=7.5 Hz, 1H), 7.51 (t, J=7.5 Hz, 2H), 7.25 (s, 1H), 7.09 (dd, J=9.0 Hz, 2.0 Hz, 1H), 7.00 (d, J=2.5 Hz, 1H), 6.89 (s, 1H), 3.97 (s, 3H), 3.90 (s, 6H), 3.85 (s, 3H). MS (ESI) calcd for C₂₅H₂₃NO₇S 481.1, found 504.1 [M+Na]⁺.

### (1-(Phenylsulfonyl)-1H-benzo[d]imidazol-2-yl)(3,4,5-trimethoxyphenyl)methanone (111)

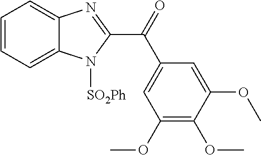

Yield: 15.2%. ¹H NMR (500 MHz, CDCl₃) δ 8.36 (d, J=7.6 Hz, 2H), 8.14 (d, J=8.3 Hz, 1H), 8.07 (d, J=7.8 Hz, 1H), 7.91 (d, J=8.1 Hz, 1H), 7.50-7.80 (m, 5H), 7.09 (s, 1H), 4.03 (s, 3H), 3.96 (s, 6H). MS (ESI) calcd for C₂₃H₂₀N₂O₆S 452.1, found 475.0 [M+Na]⁺.

### Benzo[d]thiazol-2-yl(3,4,5-trimethoxyphenyl)methanone (123a)

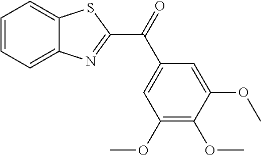

Yield: 8.4%; mp 144-146° C. ¹H NMR (CDCl₃, 500 MHz) δ 8.29 (d, J=8.0 Hz, 1 H), 8.09 (d, J=7.5 Hz, 1H), 8.03 (s, 2H), 7.63-7.67 (m, 2H), 4.06 (s, 6H), 4.04 (s, 3H). MS (ESI) calcd for C₁₇H₁₅N₂O₄S 329.1, found 352.1 [M+Na]⁺. HPLC1: t_{R} 18.4 min, purity 95.2%.

### Benzo[d]oxazol-2-yl(3,4,5-trimethoxyphenyl)methanone (123b)

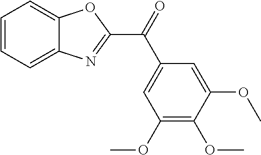

Yield: 22.5%; mp 89-91° C. ¹H NMR (CDCl₃, 500 MHz) δ 7.81-7.83 (m, 1H), 7.48-7.58 (m, 3H), 7.32-7.43 (m, 2H), 4.06 (s, 3H), 4.01 (s, 6H). MS (ESI) calcd for C₁₇H₁₅NO₅ 313.1, found 336.1 [M+Na]⁺. HPLC2: t_{R} 4.13 min, purity >97.2%.

### General procedure for the synthesis of (1H-benzo[d]imidazol-2-yl)(3,4,5-trimethoxyphenyl)methanone (118) and (1H-indol-2-yl)(3,4,5-trimethoxyphenyl)methanone (112-117, 148)

To a solution of (1-(phenylsulfonyl)-1H-indol-2-yl)(3,4,5-trimethoxyphenyl)methanones (105-110, 147) or (1-(phenylsulfonyl)-1H-benzo[d]imidazol-2-yl)(3,4,5-trimethoxyphenyl)methanone (111) (450-850 mg, 1 mmol) in ethanol (20 mL) was added sodium hydroxide (400 mg, 10 mmol, 10 equiv) and stirred overnight in darkness. The reaction mixture was diluted by 50 mL of water and extracted by ethyl acetate (250 mL). The organic layer was dried over magnesium sulfate and concentrated. The residue was purified by flash column chromatography (hexane:ethyl acetate 3:1) or recrystallized from water and methanol to give a white solid. Yield: 30-95%.

### (6-Chloro-1H-indol-2-yl)(3,4,5-trimethoxyphenyl)methanone (112)

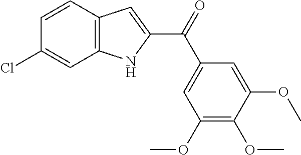

Yield: 76.8%; mp 207-209° C. ¹H NMR (CDCl₃, 500 MHz) δ 9.37 (s, 1H), 7.72 (d, J=8.5 Hz, 1H), 7.56 (s, 1H), 7.32 (s, 2H), 7.21-7.23 (m, 3H), 4.03 (s, 3H), 4.02 (s, 6H). MS (ESI) calcd for C₁₈H₁₆ClNO₄ 345.1, found 368.0 [M+Na]⁺. HPLC1: t_{R} 4.32 min, purity >99%.

### (6-Fluoro-1H-indol-2-yl)(3,4,5-trimethoxyphenyl)methanone (113)

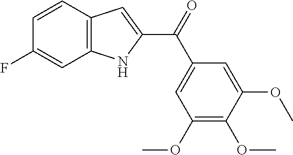

Yield: 82.5%; mp 202-204° C. ¹H NMR (CDCl₃, 500 MHz) δ 9.32 (s, 1H), 7.74 (q, J=5.0 Hz, 1H), 7.32 (s, 2H), 7.20-7.23 (m, 2H), 7.03 (dt, J=9.0 Hz, 2.0 Hz, 1H), 4.03 (s, 3H), 4.02 (s, 6H). MS (ESI) calcd for C₁₈H₁₆FNO₄ 329.1, found 352.0 [M+Na]⁺, 327.9 [M−H]⁻. HPLC1: t_{R} 4.27 min, purity 98.7%.

### (6-Methoxy-1H-indol-2-yl)(3,4,5-trimethoxyphenyl)methanone (114)

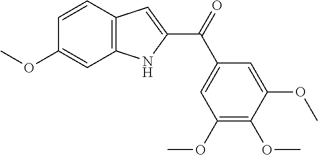

Yield: 86.9%; mp 167-169° C. ¹H NMR (CDCl₃, 500 MHz) δ 9.06 (s, 1H), 7.52 (d, J=9.0 Hz, 1H), 7.17 (s, 2H), 7.06 (s, 1H), 6.77-6.80 (m, 2H), 3.88 (s, 3H), 3.87 (s, 6H), 3.82 (s, 3H). MS (ESI) calcd for C₁₉H₁₉NO₅ 341.1, found 364.1 [M+Na]⁺. HPLC2: t_{R} 4.21 min, purity >99%.

### (5-Chloro-1H-indol-2-yl)(3,4,5-trimethoxyphenyl)methanone (115)

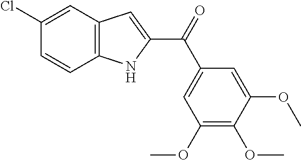

Yield: 87.2%; mp 252-254° C. ¹H NMR (DMSO-d₆, 500 MHz) δ 12.20 (s, 1H), 7.83 (s, 1H), 7.56 (d, J=8.5 Hz, 1H), 7.36 (d, J=8.5 Hz, 1H), 7.27-7.30 (m, 3H), 3.93 (s, 6H), 3.84 (s, 3H). MS (ESI) calcd for C₁₈H₁₆ClNO₄ 345.1, found 368.1 [M+Na]⁺. HPLC1: t_{R} 16.9 min, purity 96.0%.

### (6-Methyl-1H-indol-2-yl)(3,4,5-trimethoxyphenyl)methanone (116)

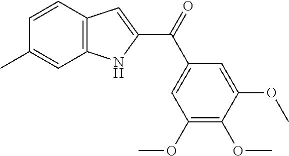

Yield: 90.1%; mp 179-181° C. ¹H NMR (CDCl₃, 500 MHz) δ 9.37 (s, 1H), 7.67 (d, J=8.5 Hz, 1H), 7.32-7.33 (m, 3H), 7.20 (s, 1H), 7.08 (d, J=8.5 Hz, 1H), 4.02 (s, 3H), 4.01 (s, 6H), 2.57 (s, 3H). MS (ESI) calcd for C₁₉H₁₉NO₄ 345.1, found 368.0 [M+Na]⁺. HPLC1: t_{R} 4.14 min, purity >99%.

### (5-Fluoro-1H-indol-2-yl)(3,4,5-trimethoxyphenyl)methanone (117)

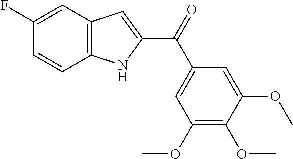

Yield: 69.8%; mp 196-198° C. ¹H NMR (CDCl₃, 500 MHz) δ 9.34 (s, 1H), 7.50 (q, J=4.5 Hz, 1H), 7.43 (dd, J=8.5 Hz, 2.0 Hz, 1H), 7.33 (s, 2H), 7.20-7.24 (m, 2H), 4.03 (s, 3H), 4.02 (s, 6H). MS (ESI) calcd for C₁₈H₁₆FNO₄ 329.1, found 352.0 [M+Na]⁺, 327.9 [M−H]⁻. HPLC1: t_{R} 4.10 min, purity >99%.

### (5-Methoxy-1H-indol-2-yl)(3,4,5-trimethoxyphenyl)methanone (148)

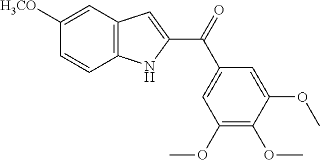

Yield: 96.3%; mp 215-217° C. ¹H NMR (DMSO, 500 MHz) δ 11.81 (s, 1H), 7.89 (d, J=9.0 Hz, 1H), 7.22 (s, 2H), 7.15-7.16 (m, 2H), 6.97 (dd, J=9.0 Hz, 2.5 Hz, 1H), 3.88 (s, 6H), 3.78 (s, 3H), 3.77 (s, 3H). MS (ESI) calcd for C₁₉H₁₉NO₅ 341.1, found 339.9 [M−H]⁻. HPLC2: t_{R} 4.18 min, purity >99%.

### (1H-benzo[d]imidazol-2-yl)(3,4,5-trimethoxyphenyl)methanone (118)

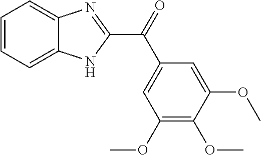

Yield: 12.7%; mp 191-193° C. ¹H NMR (CDCl₃, 500 MHz) δ 10.43 (s, 1H), 8.20 (s, 2H), 7.99 (br, 1H), 7.68 (br, 1H), 7.49 (s, 2H), 4.07 (s, 6H), 4.04 (s, 3H). MS (ESI) calcd for C₁₇H₁₆N₂O₄ 312.1, found 310.9[M−H]⁻. HPLC1: t_{R} 4.30 min, purity >99%.

### Bis(benzo[d]thiazol-2-yl)(3,4,5-trimethoxyphenyl)methanol (127)

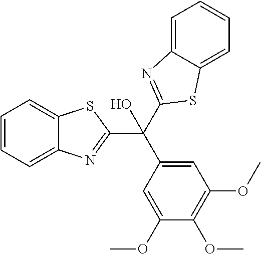

Yield: 40.5%; mp 50-52° C. ¹H NMR (CDCl₃, 500 MHz) δ 8.10 (d, J=8.0 Hz, 2 H), 7.95 (d, J=8.0 Hz, 2H), 7.55 (t, J=7.0 Hz, 2H), 7.46 (t, J=7.5 Hz, 2H), 7.23 (s, 2 H), 6.22 (s, 1H), 3.90 (s, 6H), 3.88 (s, 3H). MS (ESI) calcd for C₂₄H₂₀N₂O₄S₂ 464.1, found 487.1 [M+Na]⁺. HPLC2: t_{R} 3.99 min, purity >99%.

### General procedure for the (1-substituted-indol-2-yl)(3,4,5-trimethoxyphenyl)methanones (119-121)

To a solution of (1H-indol-2-yl)(3,4,5-trimethoxyphenyl)methanones (112, 113) (100 mg, 0.4 mmol) in THF (10 mL) in ice-bath was added sodium hydride (60% dispersion in mineral oil, 28 mg, 0.60 mmol, 1.2 equiv) followed by the addition of methyl iodide (85 mg, 0.60 mmol) (119, 121) or benzyl bromide (120) (102 mg, 0.60 mmol). The resulting reaction mixture was stirred for 5 h under reflux condition. After dilution by 50 mL of saturated NaHCO₃ solution (aqueous), the reaction mixture was extracted by ethyl acetate (100 mL). The organic layer was dried over magnesium sulfate and concentrated. The residue was purified by flash column chromatography (hexane:ethyl acetate 2:1) to give a white solid. Yield: 50%-98%.

### (6-Chloro-1-methyl-1H-indol-2-yl)(3,4,5-trimethoxyphenyl)methanone (119)

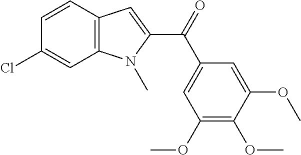

Yield: 85.3%; mp 123-125° C. ¹H NMR (CDCl₃, 500 MHz) δ 7.67 (d, J=8.0 Hz, 1H), 7.51 (s, 1H), 7.33 (s, 2H), 7.21 (d, J=8.0 Hz, 1H), 7.08 (s, 1H), 4.12 (s, 3 H), 4.02 (s, 3H), 3.98 (s, 6H). MS (ESI) calcd for C₁₉H₁₈ClNO₄ 359.1, found 382.0 [M+Na]⁺. HPLC2: t_{R} 19.5 min, purity 95.1%.

### (1-Benzyl-6-chloro-1H-indol-2-yl)(3,4,5-trimethoxyphenyl)methanone (120)

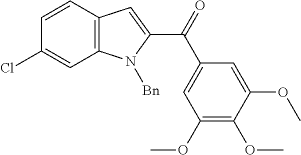

Yield: 73.4%; liquid. ¹H NMR (CDCl₃, 500 MHz) δ 7.70 (d, J=8.0 Hz, 1H), 7.48 (s, 1H), 7.41-7.43 (m, 2H), 7.28-7.37 (m, 2H), 7.17-7.23 (m, 5H), 4.02 (s, 3H), 3.96 (s, 6H). MS (ESI) calcd for C₂₅H₂₂ClNO₄ 435.1, found 458.0 [M+Na]⁺. HPLC1: t_{R}19.3 min, purity 95.3%.

### (6-Fluoro-1-methyl-1H-indol-2-yl)(3,4,5-trimethoxyphenyl)methanone (121)

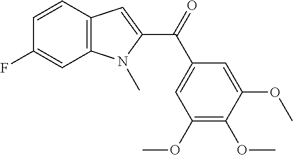

Yield: 50.8%; mp 98-100° C. ¹H NMR (CDCl₃, 500 MHz) δ 7.69 (q, J=8.5 Hz, 1H), 7.26 (s, 2H), 7.02-7.17 (m, 3H), 4.11 (s, 3H), 4.02 (s, 3H), 3.97 (s, 6H). MS (ESI) calcd for C₁₉H₁₈FNO₄ 343.1, found 344.0 [M+H]⁺. HPLC2: t_{R} 4.19 min, purity >99%.

### Example 2

### Additional Synthetic Schemes for General Compound Analogs

### Compound Analogs 14-20

Structural analogs of diindole 13 are shown in FIG. 1B. The structural analog compounds 14-20 are synthesized according to the general synthetic plan outlined in Schemes 2 through 4 shown in FIGS. 1C-1E. For analog compound 14, a variety of substituted indole rings are prepared as shown in Scheme 2. To accomplish this, a variety of N-protected indoles 33 are synthesized from commercially available reagents and brominated at the 2-indole position to produce their corresponding bromides, 34. The bromides in turn are coupled via Suzuki reaction with aldehydroboric acid 4 to yield the corresponding aldehydro-indoles 35, key intermediates in this approach.

This class of aldehydro-indoles 5A, as shown in FIG. 1D, are reacted with the 2-N-protected indole 1 under basic conditions to promote regioselective deprotonation and produce the hydroxymethylene compounds 8A in high yield. Corresponding methylketones 12A are then prepared by the oxidation of methanol linkage of compounds 8A with pyridinium dichromate (PDC) in DMF. De-protection of the N-protected groups affords a series of target indole products of basic structure A-1 incorporating a variety of different substituents at varying positions in the indole system. For example, X may be halide, —OH, —OCH₃, CH₃, NO_{R}, CN, or CF₃.

For compounds 15 and 16 in FIG. 1B, aldehydro-indoles 38 linked at the 3-indole and 39 linked at the 4-, 5-, 6- or 7-indole position are prepared by respective Suzuki reactions of bromides 36 and 37 with aldehydroboric acid 4 as shown in Scheme 4 (FIG. 1E). The bi-phenyl 17, β-naphthyl 18, substituted-aryl 19, and 3,4-methylenedioxyphenyl 20 analogs shown in FIG. 1B are synthesized as shown in the bottom of FIG. 1E, using procedures similar to those described above (Hashizume et al. Chem Pharm Bull (Tokyo), 1994, 42(10):2097-2107.). These procedures provide a rapid, reliable and high yield synthetic method for the proposed compounds.

It is contemplated that other indole derivatives substituted at C3 in the indole ring may be synthesized. For example, indole may be derivatized with a substitutent at C3 that itself comprises a substituted thiazole ring. For example compound 59, methyl 2-(1H-indole-3-carbonyl)thiazole-4-carboxylate, would have the structure:

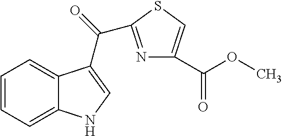

Analogs of compound 17, including compound 65, were synthesized as shown in FIG. 1i.

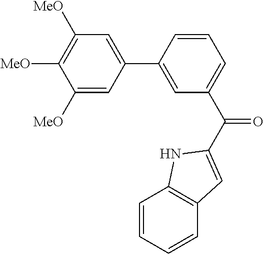

### Compound Analogs 21-23

Structural analogs 21-23, i.e., un-substituted and substituted derivatives of compounds 21, 22, and 23 (FIG. 1B) also are synthesized using the Suzuki reaction. However, in this case, the halogenated indoles are converted to lithium salts and then allowed to react with trimethoxy borate to produce the needed boric acids 3, which are then reacted with the appropriate brominated aldehydro thiophene (X═S), furan (X═O), pyrrole (X═NH), or cyclopentadiene (X═CH₂) derivative 4B to yield a variety of heterocycle-linked diindoles 5B (FIG. 1F). These derivatives are in turn to be converted to diindoles with the corresponding heterocyclic linkages using lithium diisopropylamide (LDA) and PDC as shown in Scheme 3 (FIG. 1D). Linkages in the 2, 4- and 2,5-positions will be synthesized in order to determine the importance of ring orientation and heterocyclic substitution on the benzyl linker position.

Analog 68, a trimethoxy derivative of 23, was synthesized as shown in FIG. 1J.

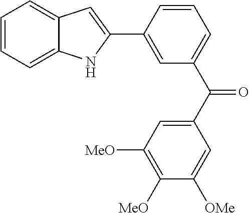

### Compound Analogs 24-28

Analogs of compounds 24 through 28 (FIG. 1B) are synthesized to determine if the methylketone linkage is absolutely required for pharmacologic activity. A variety of thioketones 24, esters 25 and 27, and amides 26 and 28 are synthesized to explore the contributions of the hydrogen bond acceptor, length of the linkage, and position of the ketone, i.e., adjacent to the benzyl linker or indole ring) to activity. Thiophene analogs of 24 are synthesized directly from their corresponding methylketone derivatives using hydrogen sulfide (FIG. 1G) (Elofson et al. J. Org. Chem. 1964, 29; Paquer, D. and Vialle, J. Bulletin de la Societe Chimique de France, 1969, 10:3595-3601.), while the ester and amide derivatives are made by reaction of the 2-amino 47 or 2-hydroxy-indoles 46 with 45 as previously described (Li et al. Chemistry, 2000, 6(9):1531-1536; Maugard et al. Phytochemistry, 2001, 58(6):897-904; Venepalli et al. J Med Chem, 1992, 32(2):374-378).

### Compound Analogs 29-31

Analog compounds of 29-31, both substituted and unsubstituted derivatives (FIG. 1B) are synthesized to determine the structure-activity relationships for tubulin inhibition, anticancer activity, transport, and hepatic metabolism. Analogs are synthesized using reaction conditions as shown in Scheme 7 (FIG. 1H) which are nearly identical to those described in Schemes 2 through 6 (FIGS. 1C-1G), with the exception that the iodo-indole 56 will be lithiated and coupled with the brominated aldehydro compound 55 to give the corresponding alcohol, which is subsequently with PDC to the methylketone 57.

### Example 3

### The Effect of Compound 68 Against Cancer Cell Lines In Vitro and In Vivo

### Materials and Methods

### Chemicals and Animals.

Monoclonal antibodies to phospho-Bcl-2 (pBcl-2), cyclin B1, Cdc25C, Cdc2, phospho-Cdc2 (pCdc2), and horseradish peroxidase conjugated secondary antibodies were purchased from Millipore Corporation (Billerica, Mass.). Bovine brain tubulin protein was purchased from Cytoskeleton, Inc. (Denver, Colo.). [³H]Vinblastine and [³H]podophyllotoxin were purchased from Moravek, Inc. (Brea, Calif.). Sephadex G25 column and Cell Death Detection ELISA (anti-histone ELISA) were purchased from Roche Applied Science (Indianapolis, Ind.). Murine 2.5S nerve growth factor was purchased from Promega (Madison, Wis.). All other chemicals were purchased from Sigma (St. Louis, Mo.).

Four to five week old male ICR mice and male nu/nu nude mice were purchased from Harlan Biosciences (Indianapolis, Ind.).

### Cell Culture.

LNCaP, PC-3, DU-145, PPC-1, TSU-Pr1, HT-29, MCF-7, K562, PC-12, HEK-293, MES-SA, and MES-SA/DX5 were originally obtained from ATCC (Rockville, Md.). All cells obtained from ATCC were immediately expanded and frozen down such that all cell lines could be restarted every 2-3 months from a frozen vial of the same batch of cells. K562/DOX, HEK293-pcDNA3-10, and HEK293-482R2 were kindly provided by a colleague. PcDNA3-10 vector and pcDNA3-10 vectors containing human MRP1 and MRP2 cDNAs were obtained from a colleague and transfected into HEK-293 cells in 2007. For the in vivo xenograft studies, PC-3, MES-SA, and MES-SA/DX5 were authenticated at Research Animal Diagnostic Laboratory (Columbia, Mo.) within four months before studies. Inter-species contamination was tested by PCR and the identity of the cell lines was verified by generating a genetic profile. For all other cell lines, authentication was not performed other than what was done by ATCC. MES-SA and MES-SA/DX5 were maintained in McCoy's 5A Medium containing 2 mM L-glutamine supplemented with 10% fetal bovine serum (FBS). PC12 was maintained in RPMI-1640 medium with 5% FBS and 10% heat-inactivated horse serum. All other cells were maintained in RPMI-1640 medium with 2 mM L-glutamine and 10% FBS.

### Growth Inhibition Assay.

The cytotoxic or anti-proliferative activity of test compounds was investigated in several cell lines using the sulforhodamine B (SRB) assay. MTT (3-(4,5-dimethylthiazol-2-yl)-2,5-diphenyltetrazolium bromide) assay was also used for the leukemia cell lines. Cultured cells were plated into 96-well plates and incubated with medium containing different concentrations of the test compounds for 96 h. Cells were stained with SRB solution or MTT solution. The optical density was determined at 540 nm on a microplate reader (Dynex Technologies, Chantilly, Va.). Plots of percent inhibition of cell growth versus drug concentration were constructed, and the concentration that inhibited cell growth by 50% relative to the untreated control (IC₅₀) was determined by nonlinear least squares regression using WinNonlin software (Pharsight Corporation, Cary, N.C.).

### Determination of DNA Fragmentation by ELISA.

Apoptosis was measured by quantitation of cytoplasmic histone-associated DNA fragments using the cell death detection ELISA kit. Cells were seeded in 6-well plates and exposed to 68 for 24 h at different concentrations. The quantitation of DNA fragments was performed according to the manufacturer's instructions. Results are expressed as the enrichment factor (i.e., the ratio of the optical density in treated cells to the optical density in control cells).

### Cell Cycle Analysis.

Cell cycle distribution was determined by propidium iodide (PI) staining. Treated cells were washed with PBS and fixed with 70% ice-cold ethanol overnight. Fixed cells were then stained with 20 μg/mL of PI in the presence of RNase A (300 μg/mL) at 37° C. for 30 min. Cell cycle distribution was analyzed by fluorescence-activated cell sorting (FACS).

### Western Blot Analysis.

Treated cells were lysed in cold lysis buffer (20 mM Tris-HCl, pH 7.5, 150 mM NaCl, 1 mM Na₂ EDTA, 1% Triton x-100, and freshly added Na₃VO₄ (2 mM), NaF (20 mM) and complete protease inhibitor cocktail). For each sample, an aliquot (20 to 40 μg) of total protein was loaded and run on a SDS-Page gel. Protein was transferred to nitrocellulose membranes (Bio-Rad Laboratories, Hercules, Calif.), blocked with 5% non-fat milk, incubated with primary antibody overnight at 4° C., and then incubated with secondary antibody at rt for 1 h. ECL™ (GE Healthcare Bio-Sciences Corp., Piscataway, N.J.) was used for detection. The band intensity was measured by densitometry using TotalLab TL100 Software v2006 (Nonlinear Dynamics Ltd., Newcastle upon Tyne, UK).

### In Vitro Tubulin Polymerization Assay.

Tubulin polymerization kits (Cytoskeleton, Denver, Colo.) were used to study tubulin depolymerization. The reaction contained 50 μL of 4 mg/mL tubulin in PEM buffer (80 mM PIPES, pH 7.0, 0.5 mM EGTA, 2 mM MgCl₂, 1 mM GTP, 10% glycerol). The procedure was done according to the manufacturer's protocol. Tubulin polymerization was monitored in a UV spectrophotometer for 40 minutes at 340 nm at 37° C.

### In Vitro Binding Assay by Spin Column.

A spin column binding assay was done as reported previously using bovine tubulin, size-exclusion Sephadex G25 columns, and ³H-labeled ligands. Briefly, tubulin (0.1 mg/mL) was incubated in PEM buffer either with 3 μM vinblastine containing [³H]vinblastine (4×10⁴ dpm/nmol) in the presence of different concentrations of vincristine or 68 or with 3 μM podophyllotoxin containing [³H]podophyllotoxin (4×10⁴ dpm/nmol) in the presence of different concentrations of colchicine or 68 at 37° C. for 1 h. Samples were then loaded onto size-exclusion Sephadex G25 columns and centrifuged at 200 g for 1 min, and radioactivity in the flow-through was analyzed by scintillation counting.

Indirect Immunofluorescence Microscopy.

PC-3 cells were plated on poly-D-lysine coated glass cover slips in 6-well plates (5×10⁴ cells/well) the day before treatment. After cells were incubated with cytotoxic agents at 37° C. for 24 h, cells were fixed on the cover slips with 4% formaldehyde in PBS for 10 min and permeabilized by 2% Triton-X in PBS for 5 min at rt. Fixed cells were blocked with 3% bovine serum albumin in TBST for 30 min and incubated with anti-α-tubulin-FITC antibody (1:100 dilution) at 4° C. overnight. Cover slips were mounted on slides using mounting medium with 4′,6-diamidino-2-phenylindole (DAPI), followed by microscopic analysis with a Zeiss Axioplan 2 fluorescent microscope (Carl Zeiss, Thornwood, N.Y.). Images were acquired with a Zeiss Axiocam HRc, using Zeiss AxioVision.

### In Vivo Antitumor Efficacy Study.

PC-3 cells (2.5×10⁶ cells/site), MES-SA cells (2×10⁶ cells/site), and MES-SA/DX5 (2×10⁶ cells/site) plus Matrigel (BD biosciences, San Jose, Calif.) were injected subcutaneously into flanks of male nu/nu mice. Tumor size was measured using calipers every 3-4 days and calculated as V=π/6×(length)×(width)². When tumors reached a volume of approximately 100˜150 mm³, drug treatment was initiated. The control group was treated with vehicle (10% DMSO in PEG300). During the treatment, tumor size and body weights were measured every 2-4 days.

### In Vitro Neurite Outgrowth.

PC-12 cells were seeded in 6-well plates coated with poly-D-lysine (BD, Franklin Lakes, N.J.) (10000 cells/well). After 6 h, cells were pretreated for 3 days by adding 100 ng/mL murine 2.5S β-NGF (Promega, Madison, Wis.) to induce neuronal differentiation and neurite outgrowth. Differentiated cells were then treated for 24 h with compounds at various concentrations in the presence of NGF (100 ng/mL). Vinblastine and docetaxel were used as positive controls. After compound treatments, approximately 100 cells in each well were randomly chosen and the numbers of cells with no neurites, short neurites (<2×cell body), and long neurites (>2×cell body) were counted (n=3).

### Rotarod Performance.

The training and rotarod test was conducted as described previously. Briefly, during the training phase for 2 consecutive days (3 trials/day), mice (10 mice/group) were trained to run in a rotarod apparatus without falling at a constant speed (12 rpm) for a maximum of 2 min. For the rotarod performance test, duration of each trial was less than 5 min. During this time, the rotation speed was constantly increased from 5 rpm to 40 rpm. Tests were performed for 2 weeks (2 days/week). The animals were tested in three trials per day with an intertrial interval of 30 min. Animal performance was recorded as the average elapsed time for the mice to fall off.

### Statistical Analysis.

The results (mean values±SD) were subjected to statistical analysis by single-factor ANOVA. The level of significance was set at P<0.05.

### Results

TABLE 1
Anticancer efficacy of 68 in different cancer cell lines and
MDR cell lines with different resistance phenotypes

| | IC₅₀ (nM) | | | |
|---|---|---|---|---|
| | 68 | Vinblastine | SN-38 | Docetaxel |
| LNCaP | 30.9 ± 3.5 | 3.4 ± 0.9 | ND | 4.7 ± 1.3 |
| PC-3 | 28.2 ± 2.0 | 1.4 ± 0.3 | ND | 6.3 ± 0.4 |
| DU-145 | 22.8 ± 4.1 | 2.6 ± 1.0 | ND | 5.2 ± 1.0 |
| PPC-1 | 18.7 ± 1.5 | 1.1 ± 0.4 | ND | 2.7 ± 1.0 |
| TSU-Pr1 | 17.6 ± 2.1 | 1.6 ± 0.1 | ND | 2.6 ± 0.9 |
| HT-29 | 15.1 ± 1.3 | 2.4 ± 0.2 | ND | 4.1 ± 0.2 |
| MCF-7 | 39.2 ± 2.9 | 1.4 ± 0.4 | ND | 3.8 ± 0.8 |
| P-gp | | | | |
| K562 | 23.6 ± 2.4 | 1.4 ± 0.4 | ND | 2.7 ± 1.0 |
| K562/Dox | 18.0 ± 4.4 | 268 ± 48 | ND | 548 ± 76 |
| | (0.8) | (191) | | (203) |
| MES-SA | 31.5 ± 2.9 | 2.3 ± 0.8 | ND | 5.9 ± 1.1 |
| MES-SA/DX5 | 32.3 ± 8.2 | 45.7 ± 5.3 | ND | 76.4 ± 8.7 |
| | (1.0) | (20) | | (13) |
| MRP | | | | |
| HEK293- | 23.8 ± 1.7 | 5.0 ± 1 | 8.4 ± 1 | 4.4 ± 2 |
| pcDNA3.1 | | | | |
| HEK293-MRP1 | 30.4 ± 6.4 | 24.3 ± 2 | 17.7 ± 1 | 31.3 ± 5 |
| | (1.3) | (4.9) | (2.1) | (7.2) |
| HEK293-MRP2 | 31.5 ± 8 | 18.1 ± 4 | 12.4 ± 1 | 33.1 ± 4 |
| | (1.3) | (3.7) | (1.5) | (7.6) |
| BCRP | | | | |
| HEK293- | 13.5 ± 1.6 | ND | 2.9 ± 0.3 | ND |
| pcDNA3-10 | | | | |
| HEK293-482R2 | 13.6 ± 1.3 | ND | 94.3 ± 20 | ND |
| | (1.0) | | (32.5) | |

NOTE: P-gp, MRP1, MRP2, BCRP were over-expressed in K562/DOX, MES-SA/DX5, HEK293-MRP1, HEK293-MRP2 and HEK293-482R2. The resistance factor (in parentheses) was calculated as the ratio of IC₅₀ values for the resistant cell subline to that of the parental cell line. All experiments were performed at least in
three replicates.
ND not determined.

### The Effect of 68 on Cell Proliferation.

To explore the effect of 68 on cancer cell proliferation, human cancer cell lines from prostate, colon, breast, and bladder were treated with different concentrations of compounds using vinblastine and docetaxel as positive controls. Cell proliferation was measured by SRB assay. Proliferation of all cell lines was inhibited by 68 in a concentration-dependent manner with IC₅₀ values ranging from 15.1 to 39.2 nM (Table 1). Vinblastine and docetaxel confirmed the assay validity by exhibiting high potency in cancer cell lines with IC₅₀ values ranging from 1.1 nM to 6.3 nM.

Since ABC transporters are thought to be one of the major causes of MDR of anticancer drugs, the antitumoral efficacy of 68 was compared with other anticancer drugs in a cytotoxicity assay using MDR cell lines with high levels of P-gp, MRP1, MRP2, or BCRP (Table 1). As judged from the resistance factor (RF, the ratio of the IC₅₀ values in cells over-expressing MDR transporters relative to the IC₅₀ values in their control cells), cells with P-gp were 13- to 203-fold resistant to vinblastine and docetaxel. RFs for vinblastine, SN-38, and docetaxel were 4.9, 2.1 and 7.2 in cells with high MRP1 expression and 3.7, 1.5, and 7.6 in cells over-expressing MRP2. Cells transfected with the vector including BCRP gene were also 33-fold resistant to SN-38. In contrast, the cytotoxicity efficacy of 68 against cancer cells was unaltered by the MDR phenotypes.

### Apoptotic Effect Induced by 68.

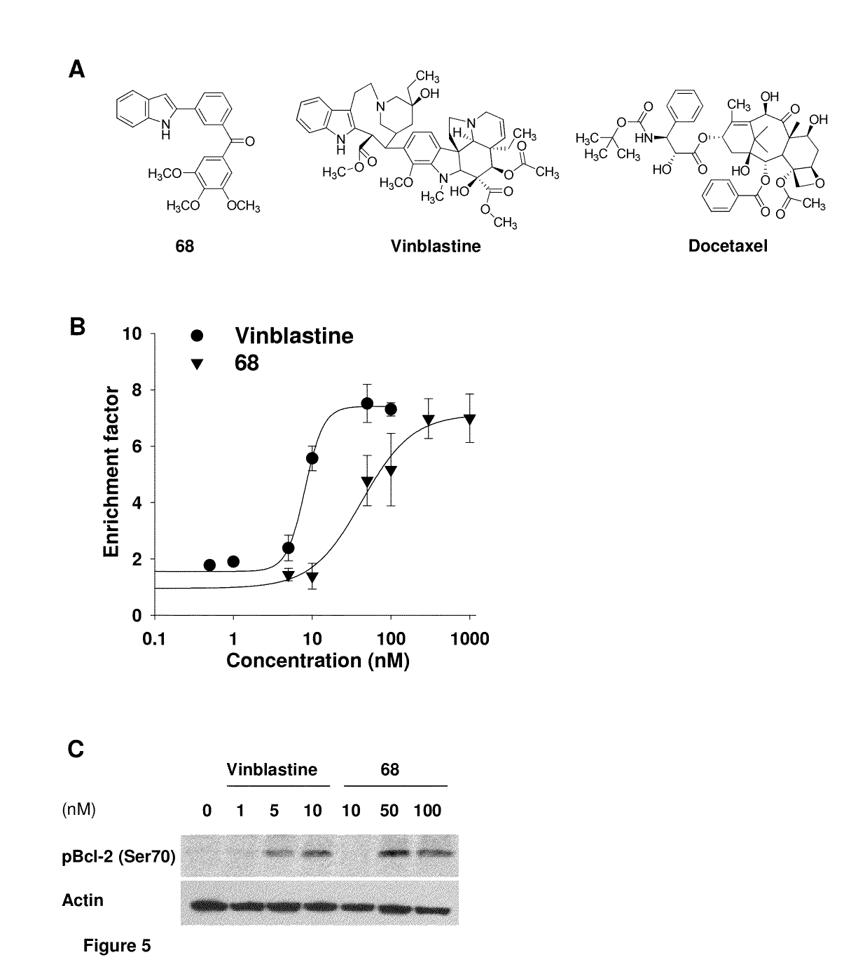

To determine whether 68 induces apoptosis of cancer cells, the DNA fragmentation was examined by using a cell death detection ELISA kit. Vinblastine was used as a positive control to confirm assay validity. Vinblastine and 68 increased the enrichment factor (absorbance of treated cells/absorbance of vehicle control cells) in a concentration-dependent manner in PC-3 cells with EC₅₀ values of 8.1 and 42.7 nM, respectively (FIG. 5B). 68 induction of Bcl-2 phosphorylation, which lead to the inactivation of the protein, was also assessed. Following 24 h treatment in PC-3 cells, immunoblots of pBcl-2 showed that 5 nM vinblastine and 50 nM 68 induced phosphorylation (Ser70) of Bcl-2. As a whole, these studies provide persuasive evidence that 68 potently induces apoptosis in PC-3 cells that is at least partially mediated by inactivation of Bcl-2.

### Effect of 68 in Cell Cycle Distribution.

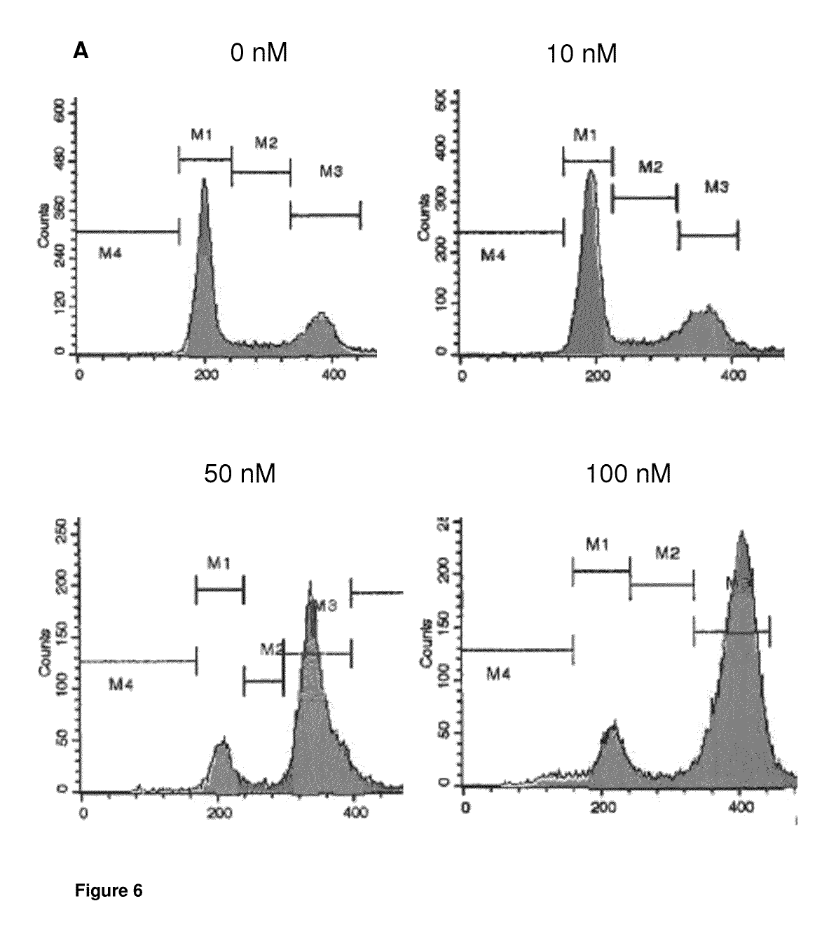

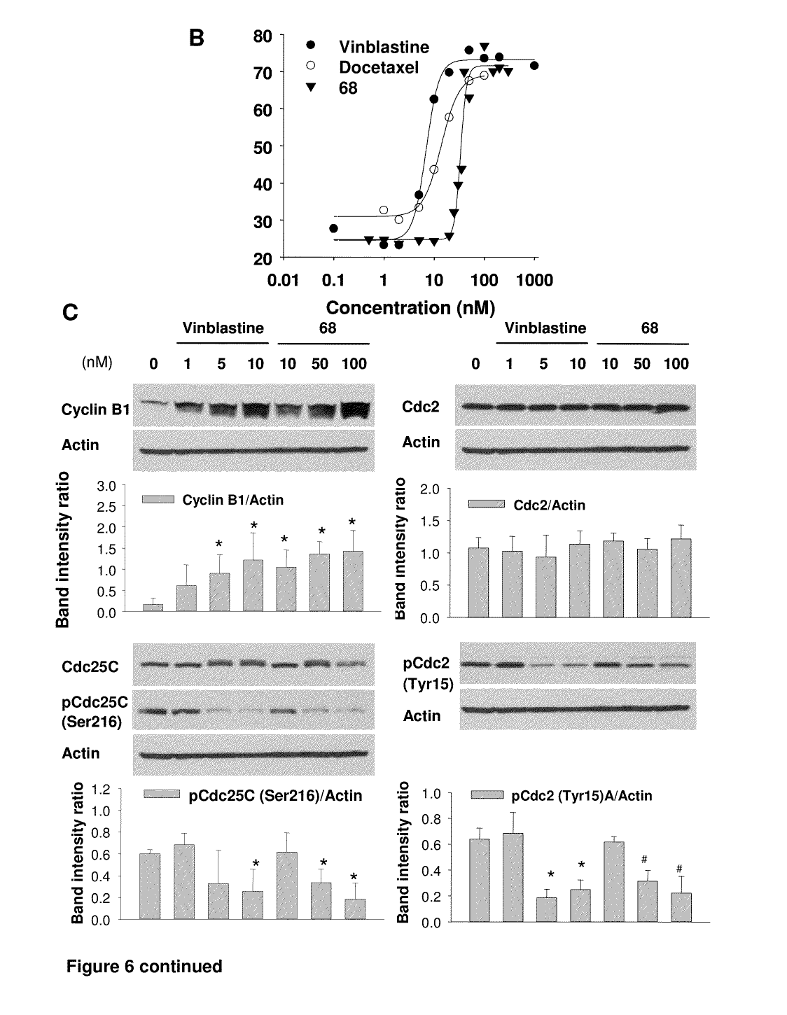

The potent antiproliferative activity and apoptosis of 68 on cancer cells prompted the test of its effects on the cell cycle. Single cells excluding debris and aggregates were selected in the study and the percentage of sub-G1 phase increased from 0.2 (control) to 0.5, 0.7, and 1.3% at 68 concentration of 10, 50, and 100 nM, respectively. The percentage of PC-3 cells in G₂M phase increased (FIG. 6A) from 19 (control) to 24, 63, and 77% at 68 concentrations of 10, 50, and 100 nM, respectively, indicating that PC-3 cells were significantly arrested in the G₂M phase in a concentration-dependent manner, a pattern that is commonly observed with taxanes and vinca alkaloids. When the percentages of cells in G₂M phase were plotted against different concentrations of the compounds (0.1 nM to 1 μM), vinblastine, docetaxel, and 68 arrested the cell cycle with EC₅₀ values of 7, 14, and 34 nM, respectively (FIG. 6B). The effect of these drugs arresting cells in the G₂M phase is closely related to the IC₅₀ values of cytotoxicity in PC-3 cells (1, 6, and 28 nM, respectively; Table 1), suggesting that this is a direct link to the mechanism of action. Therefore, changes of expressed and phosphorylated levels of key mitotic regulators following 68 treatment were evaluated. Cyclin B1 accumulation, Cdc25C dephosphorylation (Ser216), and Cdc2 dephosphorylation were observed at 68 concentrations of higher than 50 nM (24 h treatment) in PC-3 cells (FIG. 6C).

### The Effect of 68 on Microtubule Polymerization.

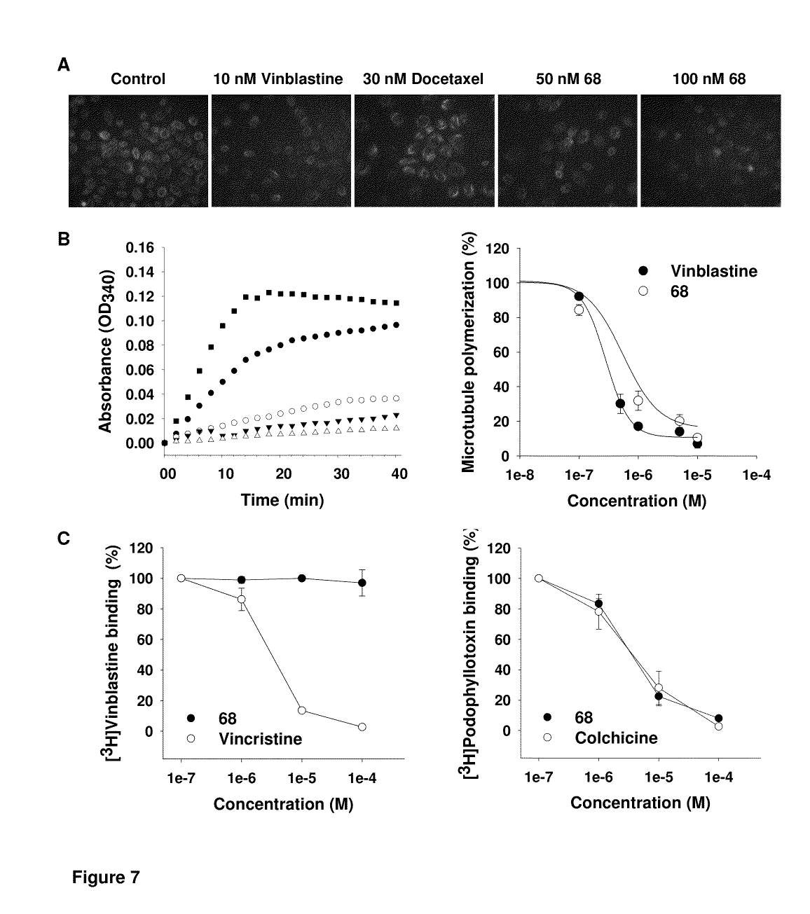

Since 68 caused cell arrest in G₂M phase, the effect of 68 on microtubule organization was investigated. In order to visualize the microtubule changes in the cells, immunofluorescence microscopy was used. As expected, vehicle-treated control cells demonstrated a variety of fluorescence patterns showing different phases of the cell cycle. On the other hand, vinblastine treated cells demonstrated the appearance of short microtubules due to fragments in the cytoplasm. In contrast, treatment with docetaxel resulted in stabilization of microtubules illustrated by an increase in the density of microtubules with brighter fluorescence. 68 treatment showed fluorescence similar to vinblastine-induced microtubule changes (FIG. 7A). Therefore, vinblastine and 68 were compared for their ability to inhibit tubulin polymerization in a cell free system using tubulin polymerization kits. As expected, vinblastine decreased the microtubule formation in a concentration-dependent manner with an IC₅₀ of 283 nM, and 68 mimicked the effects of vinca alkaloids with an IC₅₀ of 381 nM (FIG. 7B). It is generally observed that antimitotic drugs interact with tubulin either at the colchicine-, vinblastine-, and paclitaxel-binding sites. Binding studies using a spin column assay (FIG. 7C) showed that 68 inhibited [³H]podophyllotoxin binding to tubulin as colchicine did, whereas 68 was not able to displace [³H]vinblastine. This suggests that 68 directly interacts with tubulin by binding to the colchicine-binding domain.

### Efficacy of 68 in Tumor Xenograft Models.

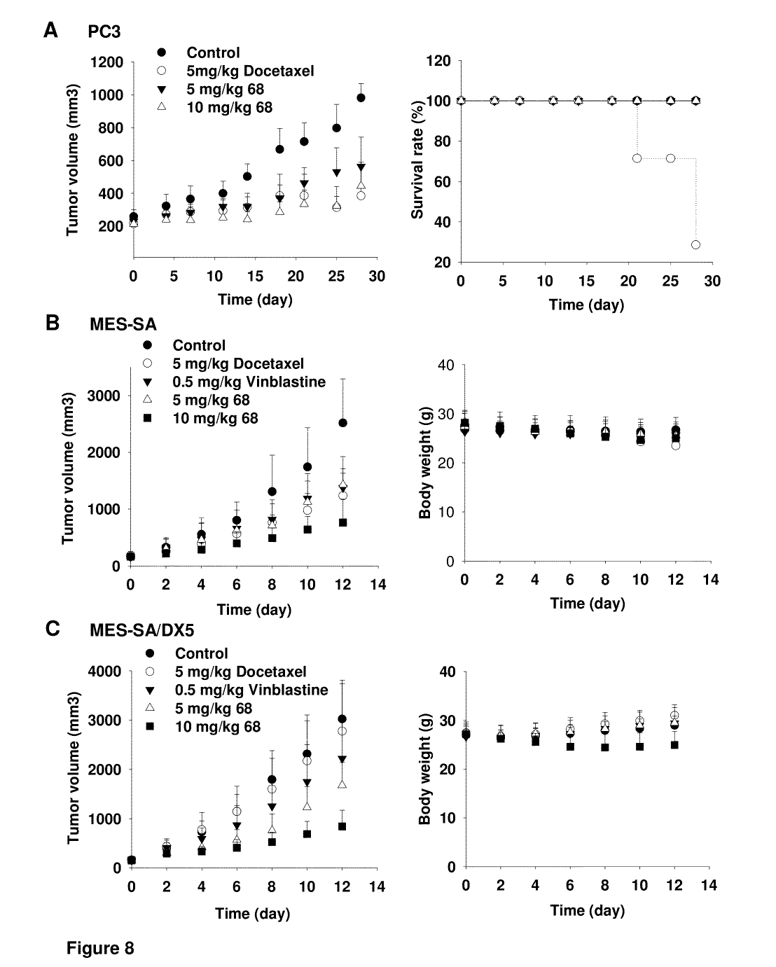

The ability of 68 to inhibit growth in PC-3, MES-SA, and MES-SA/DX5 xenograft models after i.p. injection, was examined. Docetaxel (5 mg/kg) treated mice were included as a positive control. In twice weekly treatments against PC-3 xenograft models, doses of 5 and 10 mg/kg 68 were well tolerated. Tumor growth was inhibited 36% and 68% for the 5 mg/kg and 10 mg/kg 68, respectively. The 10 mg/kg 68 group showed comparable efficacy to the 5 mg/kg docetaxel group in this regimen (FIG. 8A) without any general toxicity. In contrast, only 70% of the mice survived in the docetaxel treated group after 3 weeks with an end of study survival rate less than 30%. In the second xenograft study, the efficacy of 68 against tumors with ABC transporter related resistance was tested using MES-SA/DX5 cells over-expressing P-gp. Doses of 5 mg/kg docetaxel and 0.5 mg/kg vinblastine showed tumor growth inhibition in MES-SA xenografts with TGIs of 54% and 48%, respectively (FIG. 8B). These control compounds, however, were not effective in MES-SA/DX5 xenografts with much lower TGIs of 9% (5 mg/kg docetaxel, q2d) and 24% (0.5 mg/kg vinblastine, q2d) (FIG. 8C). In contrast, 68 inhibited tumor growth significantly in MES-SA/DX5 as well as in MES-SA xenografts with the q2d regimen (FIGS. 8B, 8C). In the MES-SA/DX5 xenograft model, 68 showed similar TGIs of 47% and 76% in the 5 and 10 mg/kg treatment groups, respectively, as compared to the MES-SA model with 47% and 74%. 68 treatment did not induce any general toxicity or significant body weight loss in either the 5 or 10 mg/kg treatment groups.

### Neurotoxicity Studies of 68

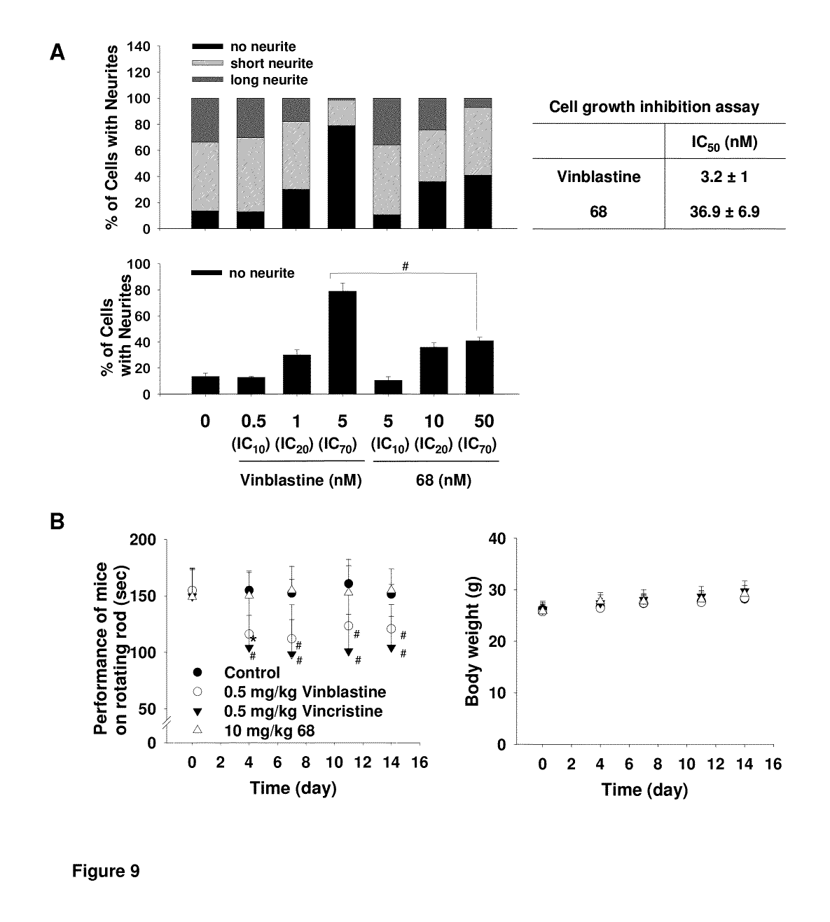

NGF-dependent neurite outgrowth is commonly used as a in vitro model to study the neurotoxic effects of drugs. NGF-dependent neurite outgrowth assay showed dose-dependent reduction of PC12 neurite extensions in the vinblastine and 68 treatment groups. 88% of vehicle treated cells expressed neurite elongation (FIG. 9A). Twenty-one percent of cells had elongated neurites when treated with 5 nM vinblastine (IC₇₀ value of vinblastine in PC-12 cell growth inhibition), but long neurites were only observed in 1.5% of cells. 68, however, resulted in higher percentages of neurite forming cells than vinblastine with 59% of cells showing neurite elongation at concentration of 50 nM 68 (IC₇₀ value of 68 in PC-12 cell growth inhibition) (P<0.01). Further, 50 nM 68, a more toxic dose than IC₅₀ value, did not reduce neurite growth showing 60% of cells with neurites (FIG. 9A). To evaluate in vivo neurotoxicity, mouse performance on the accelerating rotarod was examined following exposure to vehicle, vinblastine, vincristine, or 68. Vehicle-treated mice stayed on the rotating rod for 151 s at the end of 2 weeks, whereas 0.5 mg/kg vinblastine- and vincristine-treated mice showed significant reduction in their ability to stay on the rotating rod with mean times on the rod of 121 (P=0.0006) and 104 s (P=0.00006), respectively. 68-treated mice (10 mg/kg) did not show impaired rotarod performance as compared to the vehicle control (P=0.6) (FIG. 9B).

According to the results, compound 68, was found to inhibit cell growth in a variety of human tumor cells and arrests cell cycle progression in G₂M phase. The influence of 68 on changes of key mitotic regulators, was further examined. 68 treatment induced abnormal cyclin B1 accumulation and Cdc2 dephosphorylation in PC-3 cells. Phosphorylation of Cdc25C (Ser216) is known to induce nuclear export and reduction of activity of Cdc25C by allowing the binding of 14-3-3 to pCdc25C, while abundant phosphorylation of Cdc25C (Ser191, Ser198, etc) activates Cdc25C. Phosphorylation in Cdc25C (Ser216) decreased significantly after 68 treatment, resulting in dephosphorylation of Cdc2 (Thr14 and Tyr15) that is required for activation of Cdc2/cyclinB1 and subsequent entry into mitosis. This data suggests that 68 regulated the cell transition from O₂ to mitosis by constitutive activation of Cdc2/cyclin B1 complex. Since 68 caused cell arrest in G₂M phase, further in vitro studies were conducted to investigate whether 68 affects microtubule organization. These studies showed that 68 destabilizes microtubules in human tumor cells by binding to the colchicine-binding site. In the xenograft models using PC-3 cell line, 10 mg/kg 68 (2 days/week, i.p.) showed comparable tumor growth inhibition to 5 mg/kg docetaxel (2 days/week, i.p.) without signs of toxicity. Furthermore, 68 induced potent cell growth inhibition against drug-resistant cell lines over-expressing ABC transporters and showed potent efficacy in the animal xenograft models with MES-SA/DX5 as well as MES-SA. In contrast, 0.5 mg/kg vinblastine and 5 mg/kg docetaxel were not effective in MES-SA/DX5 resistance model as compared to sensitive MES-SA. These in vivo results suggest 68 may be effective in tumors that have obtained resistance by over-expressing P-gp. There was no apparent macroscopic toxicity in terms of body weight loss, hunched posture, diarrhea, or hematological toxicity in mouse xenograft models.

Mitotic spindle poisons leading to dynamic instability of microtubules induce tumor cell death. However, since microtubules fulfill important functions in interphase, resting, and differentiated cells for the maintenance of cytoskeletal functions and intracellular transport processes, microtubule inhibitors are known to exhibit

unwanted side effects including peripheral neuropathies. The peripheral neurotoxicity might result from a disruption of microtubule mediated axonal flow. Microtubule inhibitors such as taxanes and vinca alkaloids are also known to induce myelosuppression and neutropenia due to the inhibition of the proliferation of non-transformed cells such as hematopoietic precursor cells. A neurite outgrowth assay in PC12 cells has been proposed as an in vitro model to investigate chemotherapy-induced neuropathy. In this neurite outgrowth study, 68 caused less neurotoxic damage with higher percentages of neurite forming cells compared to vinblastine. Furthermore, since in vivo behavioral assays are also recommended to detect neuropathic pain, rotarod performance was examined. The rotarod is one of the widely used tests for neuromotor performance since 1968. The status of the motor system of ICR mice was tested on the accelerating rotarod. eqi-efficacious doses of the compounds were tested, based on tumor growth inhibition studies in mouse xenograft models. 0.5 mg/kg Vinblastine and 10 mg/kg 68 were effective in MES-SA xenografts and 0.41.6 mg/kg vincristine showed efficacy in numerous xenograft models. Vinblastine (0.5 mg/kg, q2d, i.p.) and vincristine (0.5 mg/kg, q2d, i.p.) showed impaired performance from day 4 to day 14. 68 (10 mg/kg, q2d, i.p.), in comparison, showed no loss of function, thus indicating that 68 had no effect on motor performance. Although studies are needed to elucidate the effects of 68 in the central as well as peripheral nervous system, the rotarod studies provide initial but compelling comparison of the peripheral neurotoxicity of 68 to the vinca alkaloids, due to the limited access of vinca alkaloids to the central nervous system.

Additional electrophysiologic and nerve biopsy studies would be useful to understand how 68 affects neuronal cell bodies, Schwann cells, myelins, axons, and/or nerve roots. In addition, other animal behavior studies using hot plate and von Frey fiber could be considered.

In conclusion, 68 exhibited potent anticancer activity by targeting the colchicine-binding site in a broad spectrum of human cancer cells including those that express ABC transporters without any detrimental effects on animal motor performance. This study indicates that 68 has potential against various malignancies including drug-resistant tumors.

### Example 4

### IC₅₀ of Different Cancer Cell Lines Treated with Compounds of the Invention

Materials and Methods for compounds 1 to 68

Cell viability (LNCaP, PC-3 prostate, DU145, PPC-1, and TSU-Pr1 prostate cancer cell lines, HT-29 colon cancer cell line, and MCF-7 breast cancer cell line) was quantitated using the sulforhodamine B (SRB) assay after 96 h coincubation with different concentrations of compound in 96-well plates. Cell viability of leukemia cells (K562 and doxorubicin-resistant K562/Dox) was quantitated by MTT assay after 96 h coincubation with different concentrations of compound in 96-well plates. Drug-induced apoptosis was determined by anti-histone ELISA assay and DNA laddering. Cell cycle progression was assessed by propidium iodide staining and fluorescence-activated cell sorting (FACS) analysis. In vitro tubulin polymerization assay was determined by CytoDYNAMIX ScreenTM3 (CDS-03) kits according to the manufacturer's instructions. Anti-apoptosis protein (Bcl-2 and Bcl-xl) and pro-apoptosis protein (Bax) were examined in LNCaP and PC-3 after 24 h incubation with different concentrations of compound 13 by Western blot assay. In vivo PC-3 xenograft studies were conducted by i.v. dosing of 50 mg/kg, 100 mg/kg and 150 mg/kg for 2 weeks.

Cells were plated in 96-well plates at a density of 800-5,000 cells/well, depending on the cell line, in their required growth media containing 10% fetal bovine serum. Preliminary studies were performed with each cell line using a variety of cell densities and incubation times to determine appropriate seeding densities. The compound of interest was dissolved in DMSO, diluted in cell culture medium (final DMSO concentration was less than 0.5% v/v), and added to quadruplicate wells at final concentrations ranging from 0 to 100 μM. Control wells to which only drug-free vehicle was added were included as negative controls.

Cells were incubated for 96 hour at 37° C. in a humidified atmosphere containing 5% carbon dioxide. Cell number at the end of drug treatment was quantified using the sulforhodamine B assay, as adopted by the National Cancer Institute (Rubinstein et al. J Natl Cancer Inst, 1990, 82(13):1113-1118.). Cell survival at each drug concentration was calculated as the percentage of cells present as compared to that observed in vehicle-treated control wells, and the concentration that reduced cell number by 50% relative to the untreated control (i.e., the IC₅₀) was determined by nonlinear least squares regression using WinNonLin (Pharsight Corporation).

Different concentrations of precursor indole compounds 3 and 7 and novel diindole compounds 5, 8, 10, 11, 12, 13 were also tested. The IC₅₀ for known compounds indole and di(1H-indol-3-yl)methane and the 3-yl-boronic acid analog of compound 7 also were tested.

### Materials and Methods for Compounds 68 to 127

### Cell Culture.

PC-3 was originally obtained from ATCC (Rockville, Md.) and immediately was expanded and frozen down such that all cell lines could be restarted every 2-3 months from a frozen vial of the same batch of cells. For the in vivo xenograft studies, PC-3 was authenticated at Research Animal Diagnostic Laboratory (Columbia, Mo.) within four months before studies. Inter-species contamination was tested by PCR and the identity of the cell lines was verified by generating a genetic profile. PC-3 cells were maintained in RPMI-1640 medium with 2 mM L-glutamine and 10% FBS.

### Growth Inhibition Assay.

The cytotoxic or anti-proliferative activity of test compounds was investigated in several cell lines using the sulforhodamine B (SRB) assay. Cultured cells were plated into 96-well plates and incubated with medium containing different concentrations of the test compounds for 96 h. Cells were stained with SRB solution. The optical density was determined at 540 nm on a microplate reader (Dynex Technologies, Chantilly, Va.). Plots of percent inhibition of cell growth versus drug concentration were constructed, and the concentration that inhibited cell growth by 50% relative to the untreated control (IC₅₀) was determined by nonlinear least squares regression using WinNonlin software (Pharsight Corporation, Cary, N.C.). WinNonlin was provided by a Pharsight Academic License to The Ohio State University.

### Results

TABLE 2
In Vitro IC₅₀ Values of Analogs in Various Human Cancer Cell Lines

| | | IC₅₀ (μM) | | | | | | | |
|---|---|---|---|---|---|---|---|---|---|
| ID | Structure | LNCaP | PC-3 | DU-145 | PPC-1 | TSU | CV-1 | HT-29 | MCF-7 |
| indole | 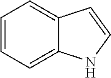 | >100 | >100 | >100 | >100 | >100 | ND | | |
| 3 | 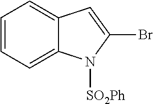 | 29.6 ± 2.2 | 69.3 ± 2.1 | 63.9 ± 1.9 | 52.1 ± 1.1 | 44.9 ± 2.0 | >100 | | |
| 5 | 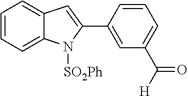 | 17.7 ± 1.2 | 23.2 ± 1.4 | 19.7 ± 1.3 | 13.9 ± 0.2 | 11.5 ± 0.1 | >100 | | |
| 7 | 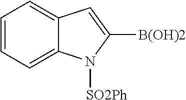 | 23.1 ± 4.0 | 73.9 ± 7.1 | 72.0 ± 3.4 | 80.8 ± 1.2 | 48.2 ± 3.4 | >100 | | |
| 128 | 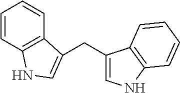 | 5.8 ± 0.5 | 39.4 ± 1.7 | 39.7 ± 2.6 | 31.8 ± 1.1 | 45.7 ± 1.1 | >100 | | |
| 129 | 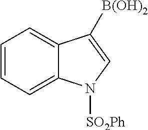 | 18.3 ± 1.4 | 59.9 ± 1.8 | 55.1 ± 2.5 | 41.1 ± 1.8 | 39.3 ± 1.6 | >100 | | |
| 130 | 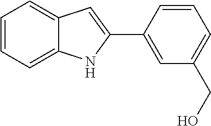 | 23.8 ± 3.0 | 65.1 ± 5.3 | 21.8 | 26.6 | ND | ND | | |
| 59 | 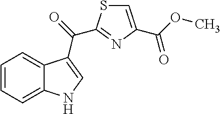 | 58.5 ± 14.3 | 90.4 ± 27.5 | ND | ND | ND | ND | | |
| 8 | 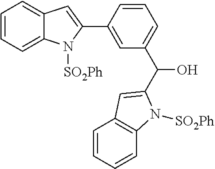 | >100 | >100 | >100 | 30 | >100 | >100 | | |
| 9 | 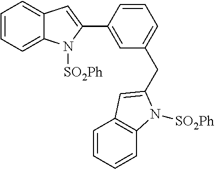 | >100 | >100 | >100 | >100 | >100 | ND | | |
| 10 | 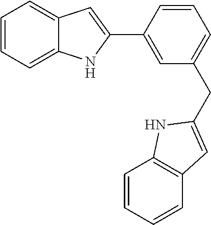 | >50 | >50 | 18.4 | >50 | 20-50 | >50 | | |
| 11 | 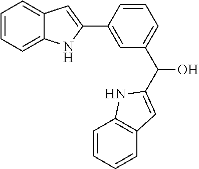 | 5.6 ± 1.1 | 13.5 ± 0.4 | 11.6 | 3.8 | ND | ND | | |
| 12 | 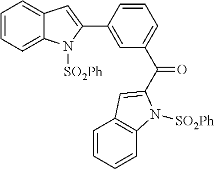 | >100 | >100 | >100 | >100 | >100 | >100 | | |
| 13 | 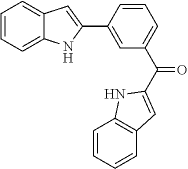 | 0.442 ± .0024 | .0811 ± .0096 | .1381 ± .0126 | .0673 ± .0011 | .0338 ± .0018 | 0.0783 ± 0.035 | 0.062 | 0.162 |
| 62 | 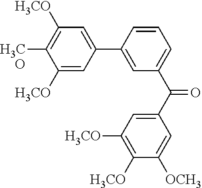 | | 40.1 | | | | | | |
| 63 | 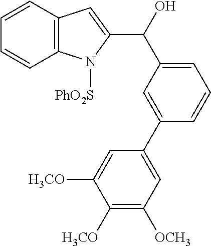 | 4.2 | 11.6 | 38 | 21.8 | | | | |
| 64 | 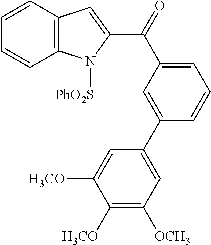 | 4.9 | 29.2 | 29.2 | 44.7 | 58.9 | | | |
| 65 | 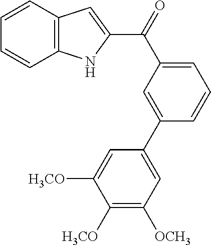 | 3.0 | 4.6 | 16.2 | 4.1 | | | | |
| 66 | 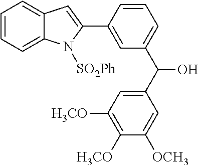 | 10.1 | 45.0 | 61.5 | 35.3 | | | | |
| 67 | 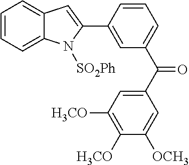 | 7.8 | 9.2 | 18 | 68.5 | | | | |
| 68 | 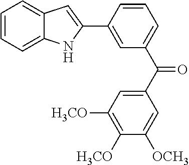 | 0.013 ± 0.0035 | 0.028 ± 0.002 | 0.023 ± 0.0041 | 0.019 ± 0.0015 | 0.018 ± 0.0021 | ND | | |
| 66A | 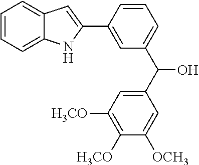 | 3.1 | 5.9 | 7.2 | 1.9 | | | | |
| 80A | 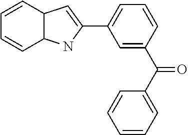 | 1.74 | 2.0 | 2.12 | 1.52 | | | | |
| 81 | 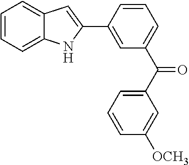 | 0.047 | 0.072 ± 0.013 | 0.129 | 0.043 | 0.042 | | 0.100 | 0.043 |
| 82 | 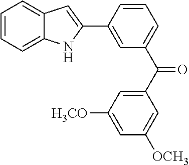 | 0.082 | 0.075 ± 0.016 | 0.134 | 0.069 | 0.065 | | 0.092 | 0.054 |
| 83 | 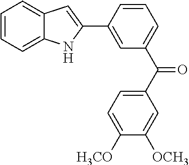 | 0.051 | 0.069 ± 0.012 | 0.131 | 0.040 | 0.044 | | 0.119 | 0.043 |
| 84 | 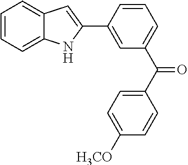 | 3.7 | 2.6 | 1.4 | 0.9 | | | | |
| 90 | 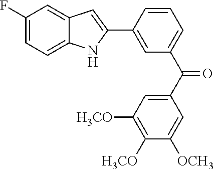 | 0.032 | 0.031 ± 0.004 | 0.028 | 0.028 | | | | 0.039 |
| 125 | 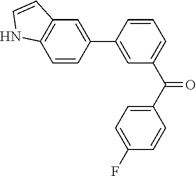 | 2.78 | 2.7 | 5.73 | 0.63 | | | | |
| 126 | 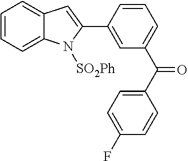 | 1.56 | 1.5 | 4.01 | 0.55 | | | | |

Table 2 shows the IC₅₀ and Ki of tested compounds against various solid tumor cell lines, including four prostate cancer cell lines (LNCaP, PC-3, DU145, PPC-1), two bladder cancer cell line (TSU-Pr1 and TCCSUP), a colon cancer cell line (HT-29), a breast cancer cell (MCF-7), and a fibroblast cell line (CV-1).

Compound 13 has an IC₅₀ significantly lower than the control compound di(1H-indol-3-yl)methane (cmpd 128). It demonstrated potent growth inhibitory effects in all of the solid tumor cell lines tested, with IC₅₀ values ranging from 34 to 138 nM (Table 2). Diindoles 10 and 11 were significantly less potent in these cell lines. IC₅₀ values for diindole 10 ranged from 0.72 μM in HT-29 cells to >50 μM in the LNCaP, PC-3, and PPC-1 cell lines. Likewise, the IC₅₀ value for diindole 11 was 5.6 and 13.5 μM in the LNCaP and PC-3 cell lines, suggesting the importance of the methanone linkage, and possibly the presence of a hydrogen bond acceptor at this position, to anticancer activity. By comparison the IC₅₀ values for paclitaxel in MCF-7 and HT-29 cells are about 2.5 nM (Rose, W. C. Taxol: Science and Applications, M. Suffness, Editor. 1995, CRC Press: Boca Raton, Fla. p. 209-235). Compound II, the indole derivative 3-(1H-indol-2-yl-)phenyl)methanol (cmpd. 130) and an indole analog methyl 2-(1H-indole-3-carbonyl)thiazole-4-carboxylate (cmpd. 59) were not yet tested (ND).

The indole-trimethoxy compound 68 demonstrated potent activity with IC₅₀ values of 18-31 nM in the cell lines tested. Compounds 80-84 explored the SAR of the trimethoxyphenyl moiety, revealing that retention of at least one of the meta methoxy groups is requisite for nM activity. This was demonstrated by 81, 82, & 83 (various combinations of m-OMe group(s)) compared with 80, 80A (no methoxy) and 84 (p-OMe). The activity of 90 was comparable to 68, demonstrating tolerance of the 5-F moiety. Replacing the trimethoxy with 4-fluoro producing only μM activity as seen in 125 and 126, but interesting these compounds demonstrated tolerance to variation in the indole attachment point.

### Example 5

### Anti-proliferative Activities of 106-107, 112-118, 119-121 and 123(a, b) and 127 Against Melanoma and Prostate Cancer Cells In Vitro

The antiproliferative activity of compounds 106-107, 112-118, 119-121 and 123(a, b) and 127 was evaluated in two human metastatic melanama cell lines (A375 and WM164) and four human prostate cancer cell lines (LNCaP, PC-3, Du 145, and PPC-1) using the methods described below. Colchicine was used as positive control. The ability of these new analogs to inhibit the growth of cancer cell lines is summarized in Table 3.

### Materials and Methods

### Cell Culture and Cytotoxicity Assay

The antiproliferative activity of compounds 106-141 were examined in two melanoma cell lines (A375 and WM-164); and four human prostate cancer cell lines (LNCaP, DU 145, PC-3, and PPC-1). These cell lines were purchased from ATCC (American Type Culture Collection, Manassas, Va.) except the PPC-1 cell line which was kindly provided by a colleague. The Pgp overexpressing multidrug-resistant cell line MDA-MB-435/LCC6MDR1 and matching sensitive parent cell line were kindly provided by a colleague. Paclitaxel resistant PC-3-TxR, DU145-TxR, and their parental cell lines were gifts provided by a colleague Melanoma cells were cultured in DMEM (Cellgro Mediatech, Inc., Herndon, Va.) and prostate cancer cells were cultured in RPMI 1640 (Cellgro Mediatech, Inc., Herndon, Va.) supplemented with 10% FBS (Cellgro Mediatech). Cultures were maintained at 37° C. in a humidified atmosphere containing 5% CO₂. 1000 to 5000 cells were plated into each well of 96-well plates depending on growth rate and exposed to different concentrations of a test compound for 48 h (fast growing melanoma cells) or 96 h (slow growing prostate cancer cells) in three to five replicates. Cell numbers at the end of the drug treatment were measured by the sulforhodamine B (SRB) assay. Briefly, the cells were fixed with 10% trichloroacetic acid and stained with 0.4% SRB, and the absorbances at 540 nm were measured using a plate reader (DYNEX Technologies, Chantilly, Va.). Percentages of cell survival versus drug concentrations were plotted, and the IC₅₀ (concentration that inhibited cell growth by 50% of untreated control) values were obtained by nonlinear regression analysis using GraphPad Prism (GraphPad Software, San Diego, Calif.).

### Results

TABLE 3
In vitro growth inhibitory effects of ABI-I compounds on melanoma and prostate cancer.

| | | | | | IC₅₀ (μM) | | | | | |
|---|---|---|---|---|---|---|---|---|---|---|
| | ID | R¹ | R² | R³ | A375 | WM164 | LNCaP | PC-3 | Du 145 | PPC-1 |
| 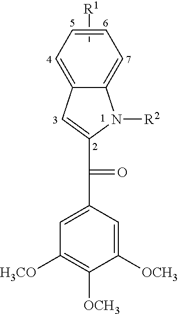 | 119 120 121 107 106 115 116 113 112 114 117 148 | 6-Cl 6-Cl 6-F 6-OMe 6-F 5-Cl 6-Me 6-F 6-Cl 6-OMe 5-F 5-OCH₃ | Me Bn Me PhSO₂ PhSO₂ H H H H H H H | NA NA NA NA NA NA NA NA NA NA NA NA | 6.65 ± 0.33 >10 5.87 ± 0.24 8.22 ± 0.81 >10 1.47 ± 0.37 >10 >10 >10 >10 2.21 ± 0.16 0.03 ± 0.01 | 8.08 ± 1.21 >10 7.50 ± 0.46 >10 >10 2.16 ± 0.26 >10 >10 >10 >10 2.26 ± 0.45 0.04 ± 0.01 | 1.04 ± 0.25 >10 1.05 ± 0.09 >10 >10 >10 >10 >10 >10 >10 8.63 ± 0.59 0.05 ± 0.02 | 3.76 ± 0.83 >10 >10 2.66 ± 0.53 >10 >10 >10 >10 >10 >10 9.16 ± 1.14 0.04 ± 0.01 | >10 >10 >10 >10 >10 >10 >10 >10 >10 >10 3.42 ± 0.17 0.08 ± 0.01 | 4.33 ± 0.72 >10 8.30 ± 1.32 1.05 ± 0.07 >10 >10 >10 >10 >10 >10 1.00 ± 0.02 0.05 ± 0.01 |
| 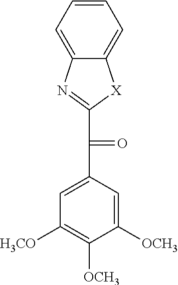 | 118 123a 123b | X = NH X = S X = O | | | >10 >10 >10 | >10 >10 >10 | >10 >10 >10 | >10 >10 >10 | >10 >10 >10 | >10 >10 >10 |
| 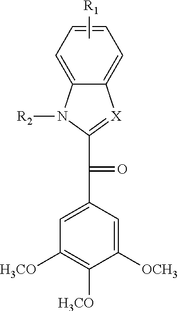 | 140 X = N 141 X = N | 6-OMe 6-F | PhSO₂ PhSO₂ | NA NA | | | >10 >10 | 2.7 >10 | >10 >10 | 1.0 >10 |
| 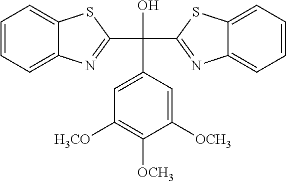 | 127 | NA | | | >10 | >10 | >10 | >10 | >10 | >10 |
| Colchicine | | | | | 0.02 ± 0.01 | 0.03 ± 0.02 | 0.02 ± 0.01 | 0.01 ± 0.01 | 0.01 ± 0.01 | 0.02 ± 0.01 |

Surprisingly, for compounds 106-141 and 148, most of the compounds were inactive (Table 3). Some of them showed moderate activity with IC₅₀ values in micromolar range as could be exemplified by compounds 119, 121, 115, 117 (1-10 μM). When comparing 119 (N-methylated-112, 5.6 μM), 120 (N-benzylated-112, >10 μM), and 112 (>10 μM), 119 is the best in this series (6-C1-indole analogues), which may suggest that the methyl group on the indole N−1 position of 119 is favorable for activity while a benzyl group on N−1 position of 120 diminishes the activity. This finding could be further confirmed by comparison of 121 (methylated-113, 1-10 μM) and 113 (>10 μM). Compound 117 (4.4 μM), with a 5-F on the indole ring, may have a more favorable binding mode over its 6-F counterpart compound 113 (>10 μM), which could be further verified by comparing 5-Cl compound 115 (1.4 and 2.1 μM in A375 and WM164, respectively) and its 6-Cl counterpart compound 112 (>10 μM), as well as the comparison between 5-OCH₃ compound 148 (0.05 μM) and its 6-OCH₃ counterpart compound 114 (>10 μM). The substantial differences in the activity of 5 and 6-substituted compounds suggest that the substitution on position-5 may have a favorable binding interaction with tubulin over the position-6. This is consistent with the literature results in which the 5-substituted analogues demonstrated excellent activity while 6-substituted compounds were generally inactive. Other ring system such as benzoxazole (123b), benzimidazole (118) and benzothiazole (123a, 127) led to inactive compounds (>10 μM).

### Example 6

### The Effect of Compound 13 Against Cancer Cell Lines In Vitro

IC₅₀ of Compounds 13, 68 and Different Drugs in K562 vs. K562/Dox Leukemia Cell Line

Cells were seeded in 96-well plates and incubated with different concentrations of compound 13, 68 or other anticancer drugs for 96 h. Cell viability was quantitated by MTT assay. The concentration that inhibited cell growth by 50% relative to the untreated control (IC₅₀) was determined by nonlinear least squares regression using WinNonLin. Table 4 is a comparison of the IC₅₀ of compounds 13, 68 and other anticancer drugs in the K562 and doxorubicin-resistant K562/DOX cell lines. The increase in IC₅₀ for compounds 13 and 68 in the doxorubicin resistant cell line is minor compared to the increases for doxorubicin, vinblastine and taxol.

TABLE 4

| | IC₅₀ (nM) | IC₅₀ (nM) |
|---|---|---|
| Compound | in K562 | in K562/DOX |
| Doxirubicin | 27.1 ± 5.5 | 859.3 ± 26.8 |
| Vinblastine | 0.3 ± 0.1 | 140.3 ± 10.1 |
| Taxol | 12.2 ± 0.2 | 1479.6 ± 479.8 |
| Cmpd 13 | 63.6 ± 2.4 | 78.2 ± 2.9 |
| Cmpd 68 | 23.6 ± 2.4 | 18.0 ± 4.4 |

### Compound 13 Induced Apoptosis and DNA Fragmentation

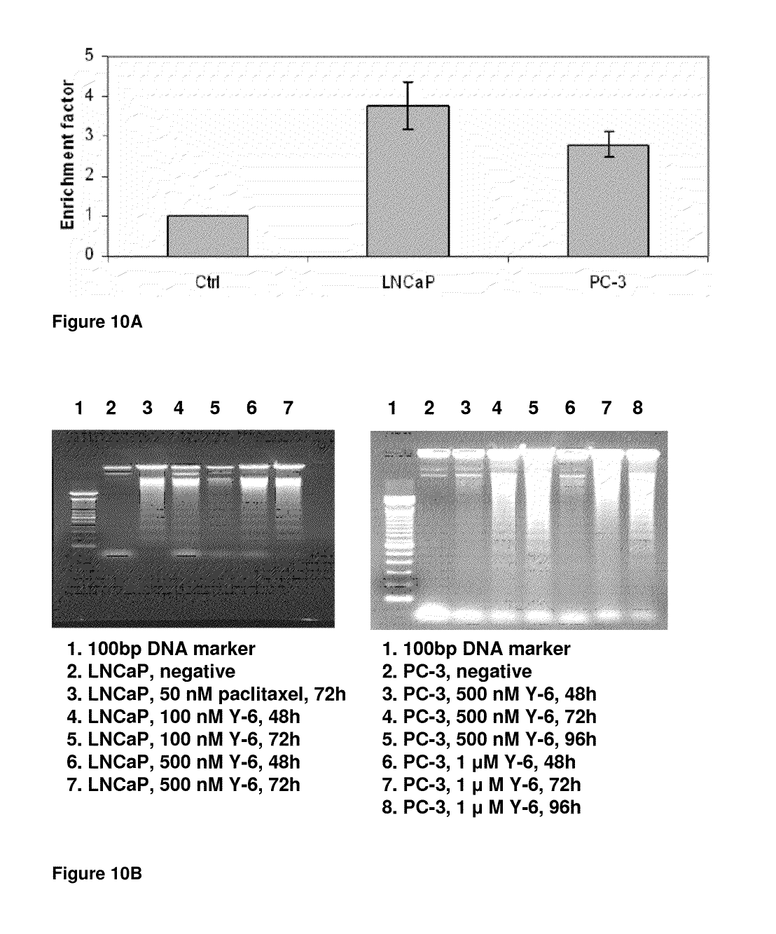

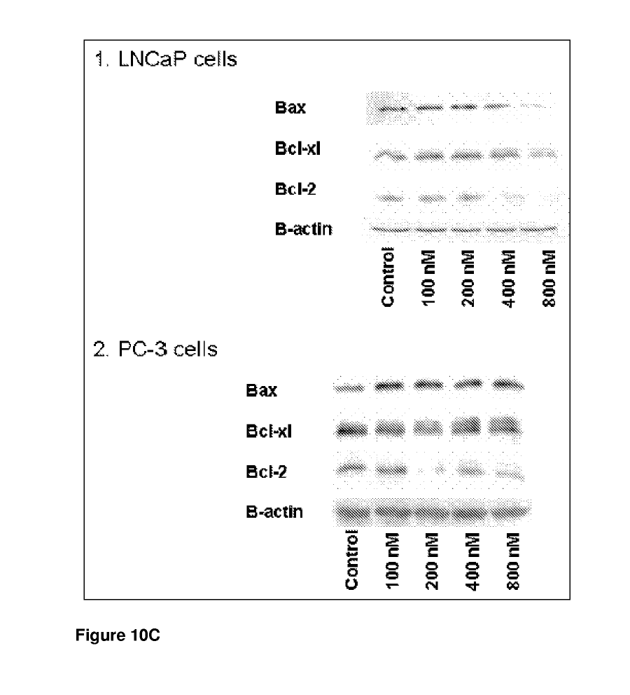

100 nM of compound 13 was incubated with LNCaP for 24 h and PC-3 for 48 h. Anti-histone ELISA detected apoptosis in the cell lines (FIG. 10A). The results are expressed as enrichment factor (Enrichment factor=OD of treated cells/OD from control cells). A Western blot of anti-apoptosis proteins, Bcl-2 and Bcl-xl, and pro-apoptosis protein Bax in LNCaP and PC-3 cells was performed. Bcl-2 was decreased by increasing concentration of compound 13 in both cell lines (FIG. 10B).

LNCaP and PC-3 cells were treated with different concentrations of drugs for different periods of time. At the end of the incubation, both floating and adherent cells were collected. Cells were lysed and low molecular weight DNA was precipitated and separated by 1.2% agarose gel electrophoresis. DNA was visualized by ethidium bromide staining and UV transillumination. Compound 13 induced DNA fragmentation in the cells (FIG. 10C).

### Compound 13 Arrests LNCaP Cells in G2/M Phase and Inhibits Tubulin Polymerization

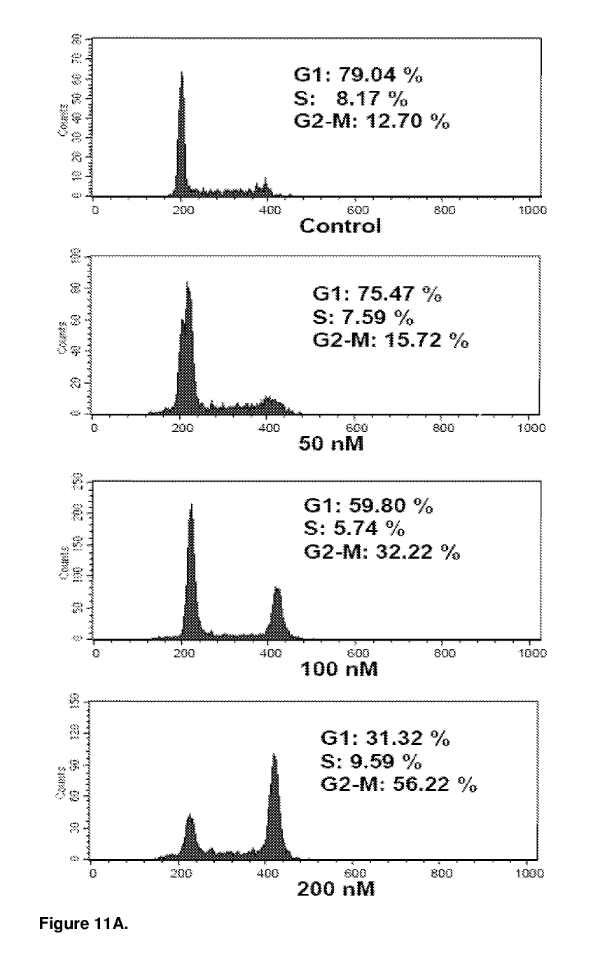

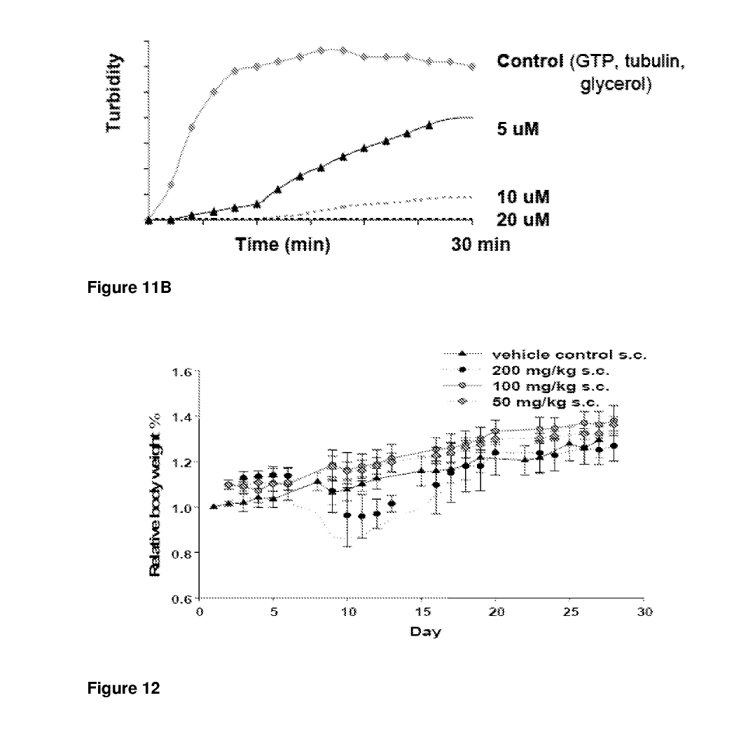

LNCaP cells were treated with 0, 50, 100 and 200 nM of compound 13 for 24 h (FIG. 11A). Cells were then harvested and fixed with 70% ethanol. Cell cycle distribution was determined by propidium iodide (P1) staining and analyzed by fluorescence-activated cell sorting (FACS) analysis.

Tubulin proteins (greater than 99% purity) were suspended (300 μg per sample) with 100 μl G-PEM buffer composed of 80 mM PIPES (piperazine-N,N′-bis(2-ethanesulfonic acid)), 2 mM MgCl₂, 0.5 mM egtazic acid and 1.0 mM guanosine triphosphate (GTP), pH 6.9, plus 5% glycerol in the absence or presence of the compound 13 at 4° C. The sample mixture was transferred to the prewarmed 96-well plate and absorbance was detected each minute for 30 minutes at 340 nm at 37° C. 20 μM of compound 13 can completely block the tubulin polymerization (FIG. 11B).

### Example 7

### The Effect of Compound 13 Against Cancer Cell Lines In Vivo

Subchronic toxicity levels of compound 13 in ICR mice

The maximally tolerated dose (MTD) in the mouse was identified. Doses of 50, 100, and 200 mg/kg (the limit of solubility in DMSO) were administered S.C. for 4 weeks (5-days on/2 days off), a commonly used regimen for initial preclinical studies of investigational anticancer agents (Rose, W. C. Taxol: Science and Applications, M. Suffness, Editor. 1995, CRC Press: Boca Raton, Fla. p. 209-235.). Body weight changes and morbidity in treated animals were used as a direct measure of toxicity. As shown in FIG. 12, all doses were generally well tolerated. There was no significant difference in morbidity or the rate of gain in body weight in animals treated with 50 or 100 mg/kg doses of diindole 13, while the highest dose caused 20% less body weight gain over the 4-week treatment period as compared to control animals treated with vehicle alone. These data suggested that diindole 13 was well tolerated, or that measurable plasma concentrations of the drug were not achieved due to rapid clearance.

### Mean Plasma Concentration-Time Profile of Compound 13 in Mice

A single dose (10 mg/kg) and various routes of administration (intravenous, oral, and subcutaneous) were used in order to approximate its in vivo disposition and interpret the results of subchronic toxicity studies and forthcoming in vivo xenograft studies. Diindole 13 was administered to groups (n=60) of mice for each route of administration. Mice (n=5 per time point) were sacrificed at up to twelve different time points (pre-dose and up to 24 h post-dose), and plasma samples were stored at −80° C. until HPLC analysis. An HPLC/UV analytical method was developed and validated to determine diindole 13 concentrations in plasma, with a linear range of 0.02 to 20 μg/mL and intra- and inter-day coefficients of variation at all concentrations less than 6%.

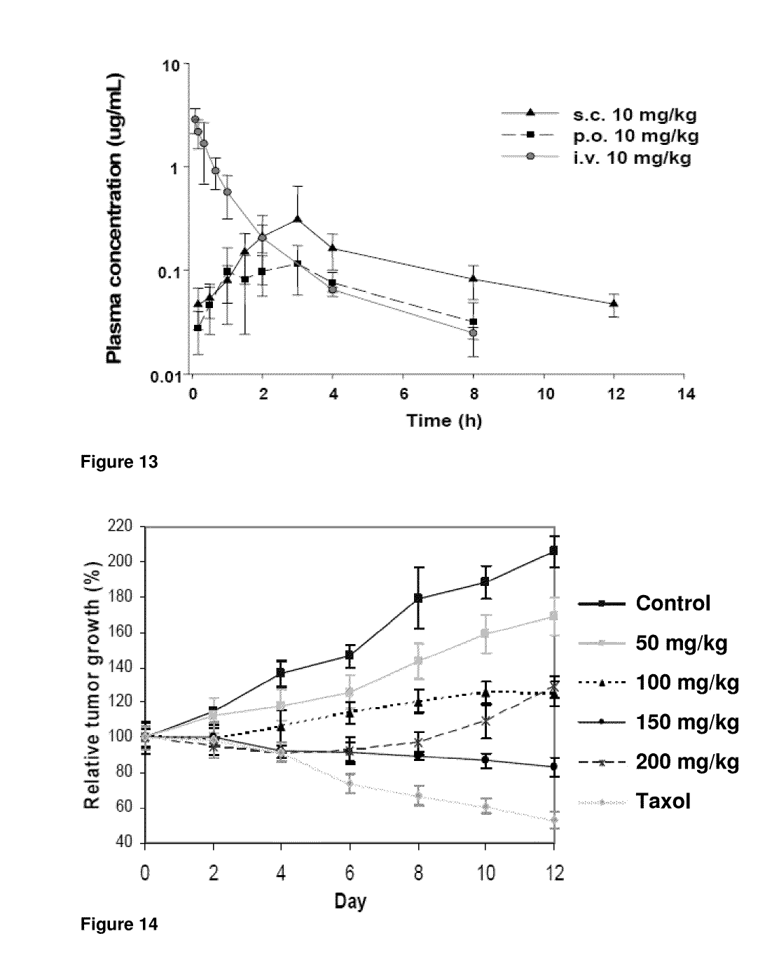

Plasma concentrations of bis-indole 13 declined rapidly after intravenous injection (FIG. 13), with a terminal half-life of less than 3 h and clearance of about 4 L/h/kg (Table 5). Urine and fecal samples collected from mice after intravenous administration of diindole 13 showed that less than 5% of the drug was excreted unchanged in urine and feces. Plasma concentrations of diindole 13 peaked at about 3 h after subcutaneous (S.C.) or oral (P.O.) administration, with absolute bioavailabilities of 73 and 29%, respectively. The terminal half-life after P.O. administration was similar to that observed after I.V. doses, but was longer after S.C. doses, likely reflecting slow absorption from the S.C. injection site due to limited aqueous solubility of diindole 13.

These data, coupled with estimates of the hepatic blood flow of the mouse (5.4 L/h/kg) (74), suggest that diindole 13 was extensively metabolized in the liver with a high hepatic extraction exceeding 0.75. Diindole 13 was widely distributed, with a volume of distribution about 10-fold larger than total body water (i.e., 0.6 L/kg). LC/MS/MS analysis of the metabolites in mice showed that diindole 13 undergoes extensive oxidative metabolism with subsequent sulfation (data not shown). Lastly, these data suggest that structural modifications, e.g., halogenation of the aromatic rings, to protect diindole 13 from microsomal oxidation via the hepatic cytochrome P450 may be beneficial. The pharmacokinetic parameters are provided in Table 5.

TABLE 5

| Parameters | I.V. | S.C. | P.O. |
|---|---|---|---|
| T_{1/2} (h) | 2.65 | 4.52 | 2.97 |
| AUC (mg*h/L) | 2.41 | 1.77 | 0.69 |
| Vₛₛ (L/Kg) | 6.35 | | |
| CL (L/h/Kg) | 4.1 | 5.7 | 14.3 |
| Tₘₐₓ (h) | 0.08 | 3.0 | 3.0 |
| Cₘₐₓ (mg/L) | 2.86 | 0.31 | 0.12 |
| F | 1.00 | 0.73 | 0.20 |

### Antitumor Activity of Compound 13 in PC-3 Xenograft Balb/c Mice

PC-3 tumor cells (2×10⁶ cells) were suspended in saline and injected S.C. in both flanks of recipient mice (n=15). Tumor size was measured every other day and volume calculated as V=π/6*(length)ₓ(width)² (75). Daily treatment (5 days on/2 days off) was initiated with diindole 13 (50, 100, or 150 mg/kg/d) or paclitaxel (15 mg/kg/d for 4 days only due to toxicity as observed by decreased body weight) when tumors reached a volume of approximately 175 mm³. Tumor growth and body weight was monitored every other day for the remainder of the study. Paclitaxel (taxol) potently suppressed PC-3 xenograft growth at a dose of 15 mg/kg/d, but also elicited significant decreases in body weight (FIG. 14). Diindole 13 also suppressed tumor growth in a dose-dependent manner, with the 150 mg/kg/d dose approaching the antitumor efficacy and toxicity of paclitaxel.

### Example 8

### In Vitro Chemosensitivity and Apoptosis Studies in Cells that Over-Express ABC Transporters

These studies use pairs of parental and stably transfected or selection-maintained cell lines. For P-glycoprotein studies, the K562 leukemia (parental) and doxorubicin-resistant K562/Dox cell lines are used. For MRPx studies, the ovarian carcinoma 2008 cell line (parental) and its stably transfected variants that over-express MRP1 (2008 MRP1), MRP2 (2008 MRP2), and MRP3 (2008 MRP3) are used. These cells were provided by Professor Anton Berns of the Netherlands Cancer Institute. For BCRP studies, the HEK-293 (parental) and its stably transfected variant that over-expresses BCRP (ABCG2) are used and are obtained through Dr. Duxin Sun from Dr. Susan Bates, NIH. The chemosensitivity, i.e., IC₅₀ values, of each active compound is determined in these cell lines pairs as an initial assessment of the ability of these transporters to influence their activity.

Pilot experiments are conducted for each cell line using different seeding densities (1×10³ to 1×10⁶ cells per well) and incubation times to optimize growth conditions. Serial ten-fold dilutions (0.01 to 100 μM) are used. If necessary, smaller ranges of appropriate concentrations near the IC₅₀ for each drug are employed. Cell number in each well is determined using the SRB or MTT, for suspension cultures like K562 assay, and IC₅₀ values are determined using nonlinear regression (WinNonlin). The extent of transport is estimated as the ratio of IC₅₀ in ABC expressing cell line/IC₅₀ in parental cell line. Known substrates, e.g., calcein, mitoxantrone, and paclitaxel, and inhibitors, e.g., verapamil, sulfinpyrazone, and fumitremorgin C, are employed to assure the viability of the expressing cell lines and confirm the contribution of the specific transporter to resistance. Statistical comparisons of IC₅₀ values between compounds will be performed using ANOVA at a 5% level of significance.

Alternatively, drug transport in these cell lines can be conducted using HPLC or LC/MS/MS to quantify analog concentration, using methods similar to those previously reported to examine the structure-activity relationships for P-glycoprotein-mediated transport of steroidal glucocorticoids (Yates et al. Pharm Res, 2003, 20(11):1794-1803). In this instance, effective permeability coefficients and transport efficiency (T_{eff}) values are used for comparison.

### Example 9

### Competition for Known Tubulin Binding Sites

A spin column binding assay, similar to that described by Bacher et al. (95-96) is used to determine whether diindoles compete for the same binding site as paclitaxel, colchicines, or vincristine. Depolymerized tubulin is incubated with radiolabeled paclitaxel, colchicine, or vincristine in the presence or absence of different concentrations (ranging from 0 to 20 μM) of unlabeled diindole 13 for 1 hour at 37° C. The incubate is then be loaded onto a size-exclusion Sephadex G25 column and centrifuged at 200×g for 1 min and the radioactivity in the flow-through will be quantified by scintillation counting. The column retains the free radioligand, but not the bound compounds. Thus, reduced radioactivity in the flow-through in the presence of diindole 13 indicates competitive binding. Unlabeled paclitaxel, colchicines, and vincristine are used as a positive controls.

Total radioactivity in each experiment is monitored for mass balance purposes. Heterocyclic or structurally modified analogs described herein that potently inhibit tubulin polymerization are used. If competition is observed, the equilibrium dissociation constant of each inhibitor (Kᵢ) for each agent is calculated by the following equation: Kᵢ=IC₅₀/(1+[L]/K_{d}), where IC₅₀ is the concentration of our ligand which inhibits the binding of ³H-radioligand by 50%, [L] is the concentration of ³H-radioligand added, and Kₐ is the equilibrium dissociation constant for the radioligand, e.g., ³H-vincristine. Experiments are performed in triplicate.

It is not expected that the binding of ³H-labeled paclitaxel, colchicine, or vincristine will be inhibited by diindole 13 or other compounds, based on the unique binding sites identified for other tubulin-interacting drugs (95-96). However, if they do, this provides another pharmacologic tool by which to examine the structure-activity relationships for tubulin interaction; namely, radioligand competition binding studies.

### Example 10

### In Vitro Hepatic Metabolism

For metabolite identification, diindole 13 and other compounds of interest are incubated with mouse liver S9 fraction (high protein concentration) with an NADPH-generating system, uridine diphosphoglucuronic acid (UDPGA) and other necessary cofactors at 37° C. for 2 h. A high protein concentration and long incubation time are chosen in order to assure maximal conversion of parent drug to metabolite(s), in the hope of identifying as many as possible, if not all, of the metabolites. Following incubation, proteins are precipitated with acetonitrile (v:v/1:1). The remaining organic phase in the supernatant is evaporated under nitrogen, and the resulting concentrated samples used for LC/MS/MS analysis.

Samples are analyzed using positive- and/or negative-ion electrospray ionization (ESI−) mass spectrometry (ThermoFinnigan LCQ DECA XP Max ion trap mass spectrometer, San Jose, Calif.). Gradient elution conditions for LC separation of the metabolites and optimized conditions for the mass spectrometer (e.g., capillary temperature, voltage, sheath and auxiliary gas flow, etc.) are determined in pilot experiments with each parent compound. Data acquisition is controlled by Xcalibur software (ThermoFinnigan) and metabolites are identified using Metabolite ID and Mass Frontier software. Synthetic standards are synthesized and independent NMR studies conducted where possible to confirm metabolite structure.

Preliminary studies using varying protein (i.e., microsome and S9) concentrations, drug concentration, and incubation time are performed to identify appropriate conditions for linear metabolite production and kinetic analyses. All reactions are conducted at 37° C. in the presence of NADPH and/or UDPGA (S9 fractions). The kinetic parameters, Km and Vmax, describing disappearance of the parent drug are determined by nonlinear regression analysis using WinNonlin (Pharsight) and the sigmoidal Emax model. Reactions are stopped by adding ice-cold acetonitrile (v:v/1:1) containing internal standard for HPLC or LC/MS/MS analysis. Protein present in the reaction mixture is precipitated by centrifugation and the supernatant either diluted with appropriate mobile phase or directly used for HPLC or LC/MS/MS analysis. HPLC and LC/MS/MS methods are developed and are validated for each analyte in each biological matrix and used for quantitation.

### Example 11

### Acute and Subchronic Toxicity (Dose-Finding) Studies

The maximally tolerated dose (MTD) and lethal dose to 10% of mice (LD₁₀) in male ICR mice (Taconic Laboratories) is determined. The analog of interest is dissolved in PEG300 or saline (as appropriate) at a concentration near its solubility, and serially diluted at 1:5 ratios to provide a range of dosing solutions. Animals receive progressively lower intravenous doses until the dose that does not result in the death or overt toxicity within 24 h is found, corresponding to the acute MTD (mg/kg). Less than 10 mice per drug are needed to establish the acute MTD.

To ensure that animal death during in vivo antitumor efficacy studies is due to tumor burden and not drug treatment, the subchronic toxicity of the analogs is determined. Mice are divided into groups of ten. Group 1 receives the acute MTD; group 2 receives 1/10 MTD; group 3: 1/25 MTD, group 4: 1/50 MTD; and group 5: 1/100 MTD. Doses are administered intravenously via the tail vein (to avoid concerns related to variable absorption after oral or subcutaneous injection) using a 5 days on/2 days off regimen for two consecutive weeks. The survival of mice is monitored for up to an additional 31 days following drug treatment. Plots of percent animals surviving versus dose (mg/kg) are constructed and the LD₁₀ determined by nonlinear regression. Studies with paclitaxel and vinblastine will also be performed.

### Example 12

### In Vivo Efficacy Against K562 and K562/Dox Tumor Xenografts

K562 and K562/Dox tumor cells (generously provided by a colleague) are mixed separately with Matrigel (Becton Dickinson) and injected subcutaneously (0.2 mL of cell and Matrigel suspension containing 1×10⁷ cells) into the left and right flank, respectively, of 8 week old male nude (nu/nu) mice. This allows one to simultaneously measure the response of both tumors to drug treatment in the same animal(s), reducing variability due to differences in body weight, pharmacokinetics, toxicity, etc. that may arise when comparing groups. Studies using tumor xenografts derived from cells that over-express other pertinent ABC transporters may also be included, if deemed pertinent.

Tumors are allowed to grow for approximately 3 weeks, with tumor volumes measured every other day, V=π/6*(length)×(width)² (75). Animals are randomized into treatment groups (n=10 per group) when tumor volumes reach 150 mm³. Ten animals per treatment group are required to assure adequate statistical power (0.8˜0.9) to identify a 25% difference in tumor volume between control and drug-treated groups. Five treatment groups are used for each compound: Group 1: un-treated control, Group 2: vehicle-treated control, Groups 3 to 5: treated with analog of interest at a daily intravenous dose of 0.01*LD₁₀, 0.1*LD₁₀, and the LD₁₀. Thus, the antitumor efficacy of each analog is examined in 50 nude mice bearing K562 and K562/Dox xenografts. Antitumor effect is assessed by measurement of tumor volume every other day during the experiments for up to 45 days after implantation, or when the tumor has reached a volume ≧10% of animal body weight. Tumor growth delay, rate of tumor growth, mean tumor volumes and final tumor volume will be compared between groups using ANOVA (α=0.05).

### Example 13

### Pharmacokinetics in Whole Animals

Male, ICR mice are used for these studies. Thirty animals receive an intravenous dose of the drug. Three mice are anesthetized and blood samples (about 500-1000 μL each) obtained via cardiac puncture or the orbital sinus at various times (up to 5 half-lives) after dosing. Plasma drug concentrations are determined using LC/MS methods (a ThermoFinnigan TSQ Quantum Discovery MAX triple quadrupole Mass Spectrometer and a LCQ Deca XP Max Ion Trap Mass Spectrometer are available in Dr. Dalton's lab, room 241). The area under the plasma drug concentration-time profile (AUC), volume of distribution, clearance and half-life is calculated for each group using nonlinear least squares regression and differences assessed using a two-tailed Student's t-test and multiple linear regression analysis. The pharmacokinetic advantage of diindole 13 and other analogs is assessed in tumor-bearing male nude nu/nu mice using a similar approach, with the exception that tumors are excised at these time points, and drug concentration in tumors containing the parental (K562) and P-glycoprotein expressing cells (K562/Dox) determined after homogenization and extraction. Maximal concentrations (Cmax) and AUCₜᵤₘₒᵣ values are compared using ANOVA.

It will be obvious to those skilled in the art that various changes may be made without departing from the scope of the invention, which is not to be considered limited to what is described in the specification.

What is claimed is:

1. A compound represented by the structure of formula IV: 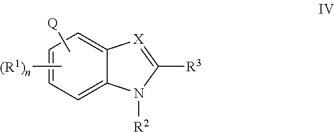
wherein:
X is CH or N;
R¹ is H, F, Cl, Br, I, CF₃, NO₂, OH, —OCH₃, CN, CH₃, alkyl, alkenyl, cycloalkyl, aryl, O-alkyl, O-aryl, or phenyl substituted at C3 or C5 with R⁴,
Q is H, F, Cl, Br, I, CF₃, NO₂, OH, —OCH₃, CN, CH₃, alkyl, alkenyl, O-alkyl, or O-aryl;
n is 0, 1, 2 or 3;
R² is H, CH₃, alkyl, benzyl, or —SO₂Ph;
R³ is phenyl substituted at C3 or C5 with R⁴; R⁸R⁹; naphthyl substituted at C5, C6, or C7 with 2-, 3- or 6-indolyl or unsubstituted, the indolyl moiety independently substituted at C1 with R², at C4, C5, or C6 with R¹ or with a combination thereof; R¹²R¹³; or 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof;
R⁴ is R⁵; C₁₋₃alkylene-R⁵; CH₂—R⁶, CH(OH)—R⁶; C(O)R⁶; CH═CH—C(R⁷)—R⁶; —C(O)—R⁷—R⁶; —O—C(R⁷)—R⁶; R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹ or R¹²R¹³;
R⁵ is OH, NO₂, NH₂, —NH—C₁₋₃alkyl, N═N═N, CN, or OR⁶;
R⁶ is H, C₁₋₃alkyl, or a 5- or 6-membered ring independently substituted at C2, C3, C4, C5, C6 or any combination thereof with R¹;
R⁷ is O, S or NH;
R⁸ is —CH₂, —CH₂OH, C═O, C═S, C═CH₂, C═NOH, C═N(NH₂);
R⁹ is H, substituted or unsubstituted indolyl, substituted or unsubstituted aryl, phenyl independently substituted at C3 with R¹⁹ and at C4 and C5 with R¹¹; thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;
R¹⁰ is H, F, Cl, Br, I, CF₃, NO₂, OH, —OCH₃, CN, CH₃, alkyl, alkenyl, cycloalkyl, aryl, O-alkyl, O-aryl, substituted or unsubstituted naphthyl or forms a dioxolyl ring with R¹¹ at C4;
R¹¹ is H, OH, or —OCH₃;
R¹² is pyrrolyl, furanyl, thienyl, or cyclopentadienyl;
R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-naphthyl or —C(O)phenyl, each independently substituted with at C2, C3, C4, C5, or C6 with R¹;
or its isomer, tautomer, pharmaceutically acceptable salt, pharmaceutical product, N-oxide, hydrate or any combination thereof.

2. The compound of claim 1, wherein said compound is represented by the structure of formula II(a): 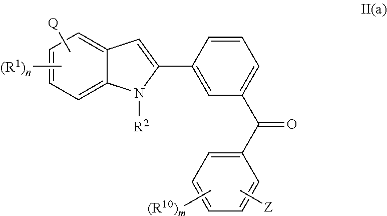
wherein:
R¹, R¹⁰, Q and Z are each independently H, F, Cl, Br, I, CF₃, NO₂, OH, —OCH₃, CN, CH₃, alkyl, alkenyl, cycloalkyl, aryl, O-alkyl or, O-aryl;
n is 0, 1, 2 or 3;
m is 0, 1, 2, 3, or 4;
R² is H, CH₃, alkyl, benzyl or —SO₂Ph.

3. The compound of claim 1, wherein said compound is represented by the structure of formula IV(a): 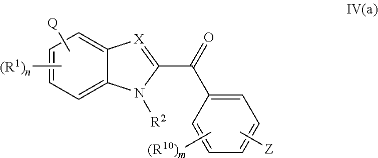
wherein:
X is CH or N;
R¹, R¹⁰, Q and Z are each independently H, F, Cl, Br, I, CF₃, NO₂, OH, —OCH₃, CN, CH₃, alkyl, alkenyl, cycloalkyl, aryl, O-alkyl or, O-aryl;
n is 0, 1, 2 or 3;
m is 0, 1, 2, 3, or 4; and
R² is H, CH₃, alkyl, benzyl or —SO₂Ph.

4. A compound represented by the structure of formula V: 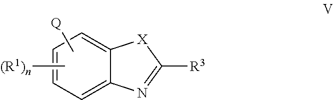
wherein:
X is CH₂, NH, N(R²), O, S, SO or SO₂;
R¹ is H, F, Cl, Br, I, CF₃, NO₂, OH, —OCH₃, CN, CH₃, alkyl, alkenyl, cycloalkyl, aryl, O-alkyl, O-aryl, or phenyl substituted at C3 or C5 with R⁴,
Q is H, F, Cl, Br, I, CF₃, NO₂, OH, —OCH₃, CN, CH₃, alkyl, alkenyl, O-alkyl, or O-aryl;
n is 0, 1, 2 or 3;
R² is H, CH₃, alkyl, benzyl, or —SO₂Ph;
R³ is phenyl substituted at C3 or C5 with R⁴; R⁸R⁹; naphthyl substituted at C5, C6, or C7 with 2-, 3- or 6-indolyl or unsubstituted, the indolyl moiety independently substituted at C1 with R², at C4, C5, or C6 with R¹ or with a combination thereof; R¹²R¹³; or 2-, 3- or 6-indolyl substituted at C1, C2, or C3 with 2-, 3- or 6-indolyl, either of the indolyl moiety independently substituted at C1 with R², at C4, C5, or C6 with R¹ or with a combination thereof;
R⁴ is R⁵; C₁₋₃alkylene-R⁵; CH₂—R⁶, CH(OH)—R⁶; C(O)R⁶; CH═CH—C(R⁷)—R⁶; —C(O)—R⁷—R⁶; —O—C(R⁷)—R⁶; R⁷R⁸-(2-, 3-, or 6-indolyl); R⁸-(2-, 3- or 6-indolyl), the indolyl moiety independently substituted at C1 with R², at C4, C5 or C6 with R¹ or with a combination thereof; R⁸R⁹ or R¹²R¹³;
R⁵ is OH, NO₂, NH₂, —NH—C₁₋₃alkyl, N═N═N, CN, or OR⁶;
R⁶ is H, C₁₋₃alkyl, or a 5- or 6-membered ring independently substituted at C2, C3, C4, C5, C6 or any combination thereof with R¹;
R⁷ is O, S or NH;
R⁸ is —CH₂, —CH(OH), C═O, C═S, C═CH₂, C═NOH, C═N(NH₂);
R⁹ is H, substituted or unsubstituted indolyl, substituted or unsubstituted aryl, thiazolyl substituted at C4 with —C(O)OCH₃ or naphthyl substituted at C5, C6, or C7 with 2-, 3- or 6-indolyl or unsubstituted, the indolyl moiety independently substituted at C1 with R², at C4, C5, or C6 with R¹ or with a combination thereof;
R¹⁰ is H, F, Cl, Br, I, CF₃, NO₂, OH, —OCH₃, CN, CH₃, alkyl, alkenyl, cycloalkyl, aryl, O-alkyl, O-aryl, substituted or unsubstituted naphthyl or forms a dioxolyl ring with R¹¹ at C4;
R¹¹ is H, OH, or —OCH₃;
R¹² is pyrrolyl, furanyl, thienyl, or cyclopentadienyl;
R¹³ is —C(O)-2-, 3-, or 6-indolyl, —C(O)-imidazole, —C(O)-thiazole, —C(O)-oxazole, —C(O)-isoxazole, —C(O)-benzoxazole, —C(O)-pyrrole, —C(O)-furan, —C(O)-oxazoline, —C(O)-oxazolidine, —C(O)-oxadiazole, C(O)-naphthyl or —C(O)phenyl, each independently substituted with at C2, C3, C4, C5, or C6 with R¹;
or its isomer, tautomer, pharmaceutically acceptable salt, pharmaceutical product, N-oxide, hydrate or any combination thereof.

5. The compound of claim 4, wherein said compound is represented by the structure of formula V(a): 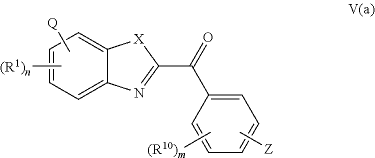
wherein:
X is CH₂, NH, N(R²), O, S, SO or SO₂;
R¹, R¹⁰, Q and Z are each independently H, F, Cl, Br, I, CF₃, NO₂, OH, —OCH₃, CN, CH₃, alkyl, alkenyl, cycloalkyl, aryl, O-alkyl or, O-aryl;
R² is H, CH₃, alkyl, benzyl, or —SO₂Ph;
n is 0, 1, 2 or 3; and
m is 0, 1, 2, 3, or 4.

6. The compound as claimed in claim 1, wherein X is CH, R¹ is H or F; R² is H or SO₂Ph; R³ is phenyl substituted at C3 or C5 with R⁴, R⁴ is R⁸R⁹, R⁸ is C═O, and R⁹ is substituted or unsubstituted aryl, independently substituted at C3, C4, C5 or any combination thereof with OCH₃, H or F.

7. The compound according to claim 6, wherein said compound is represented by the structure:

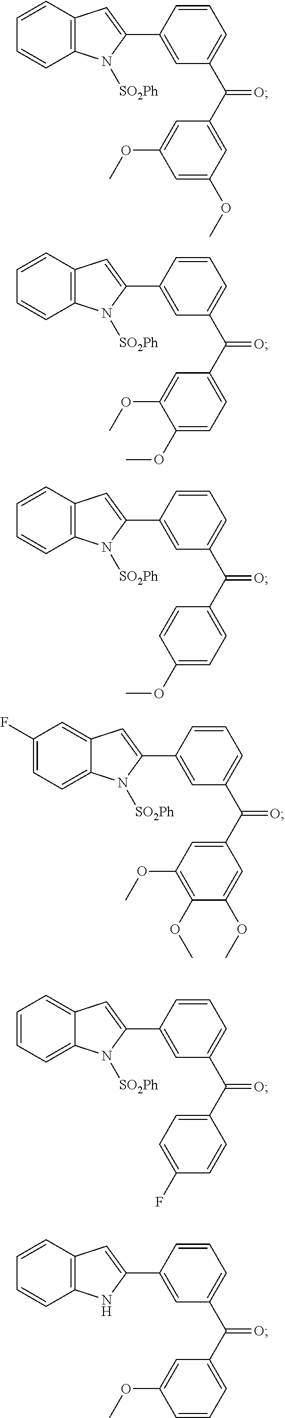

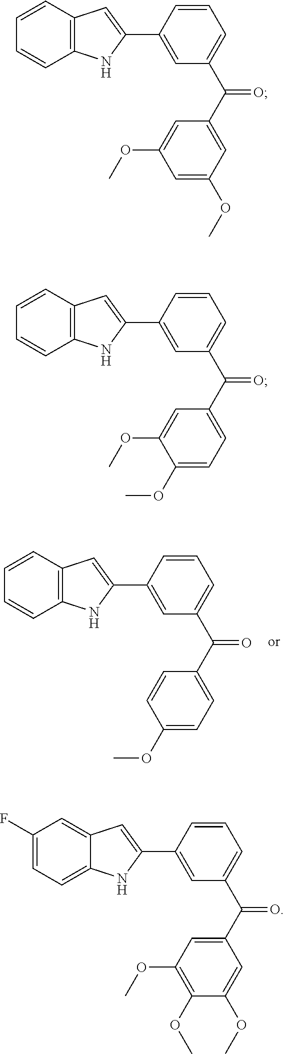

8. The compound as claimed in claim 1, wherein X is CH, R¹ is H or F; R² is H or SO₂Ph; R³ is phenyl substituted at C3 or C5 with R⁴, R⁴ is R⁸R⁹, R⁸ is CH(OH), R⁹ is substituted or unsubstituted aryl, independently substituted at C3, C4, C5 or any combination thereof with OCH₃ or H.

9. The compound according to claim 8, wherein said compound is represented by the structure:

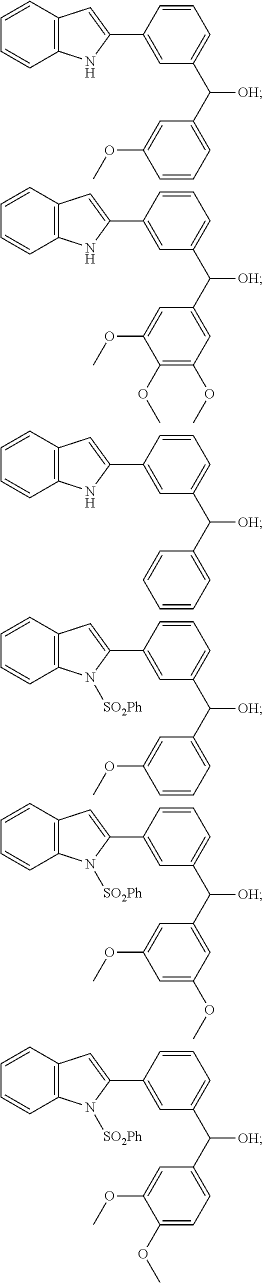

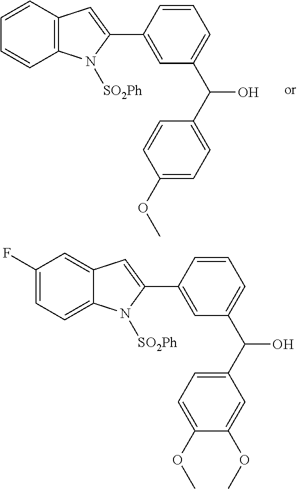

10. The compound as claimed in claim 1, wherein X is CH or N, R¹ is H, F, Cl, OCH₃, or CH₃; R² is H, SO₂Ph, CH₃ or benzyl; R³ is R⁸R⁹; R⁸ is C═O, R⁹ is substituted or unsubstituted aryl, independently substituted at C3, C4, C5 or any combination thereof with OCH₃.

11. The compound according to claim 10, wherein said compound is represented by the structure:

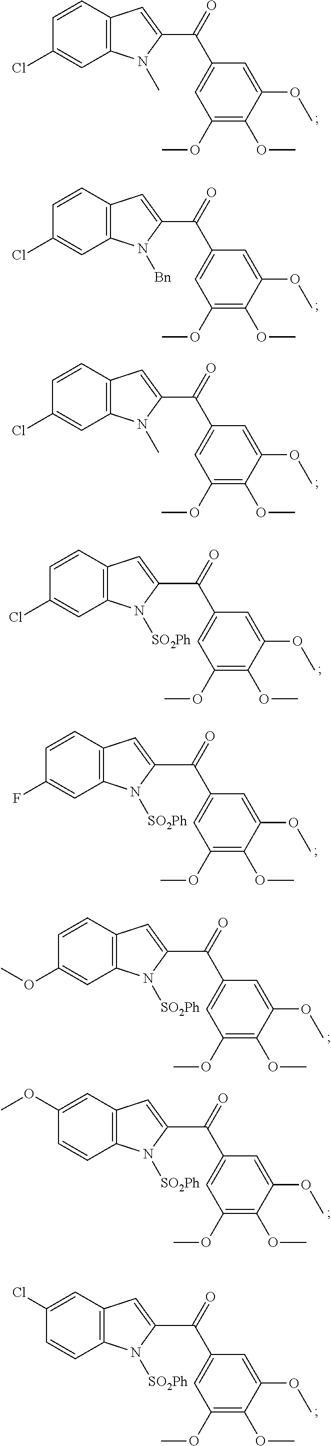

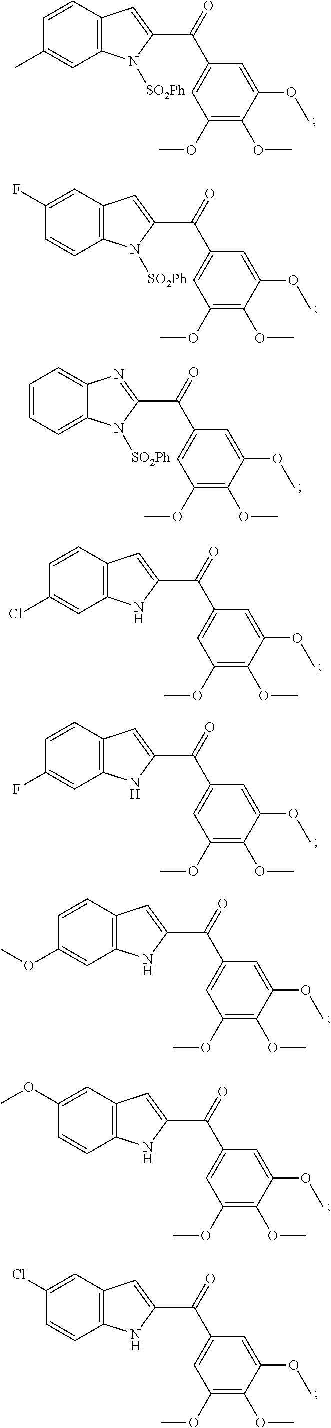

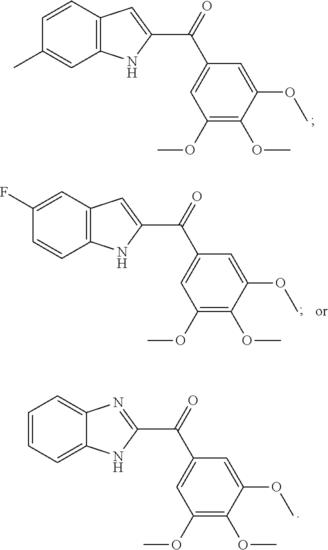

12. The compound as claimed in claim 4, wherein X is S or O, R¹ is H; R³ is R⁸R⁹; R⁸ is C═O, R⁹ is substituted or unsubstituted aryl, independently substituted at C3, C4, C5 or any combination thereof with OCH₃.

13. The compound according to claim 12, wherein said compound is represented by the structure:

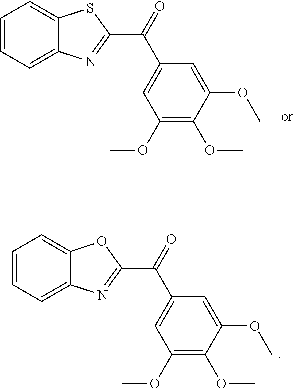

14. A pharmaceutical composition comprising a compound according to claim 1, and a pharmaceutically acceptable carrier, diluent or salt or any combination thereof.

15. A method of inhibiting tubulin polymerization in a cell associated with a cell proliferative disease, comprising administering a compound according to claim 1 to a subject, in an amount effective to inhibit tubulin polymerization in said cell.

16. The method of claim 15, wherein said cell proliferative disease is a cancer.

17. The method of claim 16, wherein said cancer is prostate cancer, melanoma, colon cancer, bladder cancer or breast cancer.

18. A method of treating, halting, suppressing, reducing the severity, reducing the incidence of, reducing the risk, causing the regression of, or inhibiting prostate cancer in a subject comprising the step of administering to said subject a compound according to claim 1, in an amount effective to treat, halt, suppress, reduce the severity, reduce the incidence of, reduce the risk of, cause the regression of, or inhibit said prostate cancer.

19. The method according to claim 18, wherein said prostate cancer is drug resistant prostate cancer, multidrug-resistant (MDR) prostate cancer, castration-resistant prostate cancer, metastatic prostate cancer, advanced prostate cancer or any combination thereof.

20. A method of treating, halting, suppressing, reducing the severity, reducing the incidence of, reducing the risk, causing the regression of, or inhibiting melanoma in a subject comprising the step of administering to said subject a compound according to claim 1, in an amount effective to treat, halt, suppress, reduce the severity, reduce the incidence of, reduce the risk of, cause the regressions of, or inhibit said melanoma.

21. The method according to claim 20, wherein said melanoma is resistant melanoma, multidrug-resistant (MDR) melanoma, metastatic melanoma, or any combination thereof.

22. A method of treating, halting, suppressing, reducing the severity, reducing the incidence of, reducing the risk, causing the regression of, or inhibiting a drug-resistant tumor in a subject comprising the step of administering to said subject a compound according to claim 1, in an amount effective to treat, halt, suppress, reduce the severity, reduce the incidence of, reduce the risk of, cause the regressions of, or inhibit said drug-resistant tumor.

23. The method according to claim 22, wherein said drug-resistant tumor is prostate cancer tumor, melanoma tumor, breast cancer tumor, bladder cancer tumor or colon cancer tumor.

24. A method of treating, halting, suppressing, reducing the severity, reducing the incidence of, reducing the risk, causing the regression of, or inhibiting breast cancer in a subject comprising the step of administering to said subject a compound according to claim 1, in an amount effective to treat, halt, suppress, reduce the severity, reduce the incidence of, reduce the risk of, cause the regressions of, or inhibit said breast cancer.

25. The method according to claim 24, wherein said breast cancer is drug resistant breast cancer, multidrug-resistant (MDR) breast cancer, metastatic breast cancer, or any combination thereof.

26. A method of treating, halting, suppressing, reducing the severity, reducing the incidence of, reducing the risk, causing the regression of, or inhibiting colon cancer in a subject comprising the step of administering to said subject a compound according to claim 1, in an amount effective to treat, halt, suppress, reduce the severity, reduce the incidence of, reduce the risk of, cause the regressions of, or inhibit said colon cancer.

27. The method according to claim 26, wherein said colon cancer is drug resistant colon cancer, multidrug-resistant (MDR) colon cancer, metastatic colon cancer, or any combination thereof.

28. A method of treating, halting, suppressing, reducing the severity, reducing the incidence of, reducing the risk, causing the regression of, or inhibiting bladder cancer in a subject comprising the step of administering to said subject a compound according to claim 1, in an amount effective to treat, halt, suppress, reduce the severity, reduce the incidence of, reduce the risk of, cause the regressions of, or inhibit said bladder cancer.

29. The method according to claim 28, wherein said bladder cancer is drug resistant bladder cancer, multidrug-resistant (MDR) bladder cancer, metastatic bladder cancer, or any combination thereof.
